SEQUENCE LISTING (76 sequences)

SEQ ID NO: 1 - RNA, 30 nt, synthetic construct
  source 1..30: mol_type = other RNA
  modified_base 25: mod_base = i
  modified_base order(1..3,12,14,16,26,28,29,30): mod_base = OTHER; note = 2-prime-methoxyribose
  modified_base order(1,11,14,25,29): mod_base = OTHER; note = Mesyl phosphoroamidate linkage
  modified_base order(4..5,8..9,11,13,15,17..19,22,27): mod_base = OTHER; note = 2-prime-deoxy-2-prime-fluororibose
  modified_base order(6..7,10,20..21): mod_base = OTHER; note = 2-prime-MOEribose
  modified_base order(23,25): mod_base = OTHER; note = deoxyribose
  modified_base 24: mod_base = OTHER; note = beta-homoDNA
  modified_base order(2..5,8..9,13,16..19,22..24,27..28): mod_base = OTHER; note = Phosphorothioate linkage
  modified_base 6: mod_base = OTHER; note = 5-methyl-cytidine
  modified_base order(6..7,10,12,15,20..21,26): mod_base = OTHER; note = Phosphate linkage
  modified_base order(10,20..21,23): mod_base = OTHER; note = Thymine
cccagcagcttcagtccctttctcntcgat

SEQ ID NO: 2 - RNA, 30 nt, synthetic construct; identical to SEQ ID NO: 1
  source 1..30: mol_type = other RNA
  modified_base 25: mod_base = i
  modified_base order(1..3,12,14,16,26,28..30): mod_base = OTHER; note = 2-prime-methoxyribose
  modified_base order(1,11,14,25,29): mod_base = OTHER; note = Mesyl phosphoroamidate linkage
  modified_base order(4..5,8..9,11,13,15,17..19,22,27): mod_base = OTHER; note = 2-prime-deoxy-2-prime-fluororibose
  modified_base order(6..7,10,20..21): mod_base = OTHER; note = 2-prime-MOEribose
  modified_base order(23,25): mod_base = OTHER; note = deoxyribose
  modified_base 24: mod_base = OTHER; note = beta-homoDNA
  modified_base order(2..5,8..9,13,16..19,22..24,27..28): mod_base = OTHER; note = Phosphorothioate linkage
  modified_base 6: mod_base = OTHER; note = 5-methyl-cytidine
  modified_base order(6..7,10,12,15,20..21,26): mod_base = OTHER; note = Phosphate linkage
  modified_base order(10,20..21,23): mod_base = OTHER; note = Thymine

SEQ ID NO: 3 - RNA, 30 nt, synthetic construct; identical to SEQ ID NO: 1
  source 1..30: mol_type = other RNA
  modified_base 25: mod_base = i
  modified_base order(1..3,12,14,16,26,28..30): mod_base = OTHER; note = 2-prime-methoxyribose
  modified_base order(1,11,14,25,29): mod_base = OTHER; note = Mesyl phosphoroamidate linkage
  modified_base order(4..5,8..9,11,13,15,17..19,22,27): mod_base = OTHER; note = 2-prime-deoxy-2-prime-fluororibose
  modified_base order(6..7,10,20..21): mod_base = OTHER; note = 2-prime-MOEribose
  modified_base order(23,25): mod_base = OTHER; note = deoxyribose
  modified_base 24: mod_base = OTHER; note = beta-homoDNA
  modified_base order(2..5,8..9,13,16..19,22..24,27..28): mod_base = OTHER; note = Phosphorothioate linkage
  modified_base 6: mod_base = OTHER; note = 5-methyl-cytidine
  modified_base order(6..7,10,12,15,20..21,26): mod_base = OTHER; note = Phosphate linkage
  modified_base order(10,20..21,23): mod_base = OTHER; note = Thymine

SEQ ID NO: 4 - RNA, 30 nt, synthetic construct; identical to SEQ ID NO: 1
  source 1..30: mol_type = other RNA
  modified_base 25: mod_base = i
  modified_base order(1..3,12,14,16,26,28..30): mod_base = OTHER; note = 2-prime-methoxyribose
  modified_base order(1,11,14,25,29): mod_base = OTHER; note = Mesyl phosphoroamidate linkage
  modified_base order(4..5,8..9,11,13,15,17..19,22,27): mod_base = OTHER; note = 2-prime-deoxy-2-prime-fluororibose
  modified_base order(6..7,10,20..21): mod_base = OTHER; note = 2-prime-MOEribose
  modified_base order(23,25): mod_base = OTHER; note = deoxyribose
  modified_base 24: mod_base = OTHER; note = beta-homoDNA
  modified_base order(2..5,8..9,13,16..19,22..24,27..28): mod_base = OTHER; note = Phosphorothioate linkage
  modified_base 6: mod_base = OTHER; note = 5-methyl-cytidine
  modified_base order(6..7,10,12,15,20..21,26): mod_base = OTHER; note = Phosphate linkage
  modified_base order(10,20..21,23): mod_base = OTHER; note = Thymine

SEQ ID NO: 5 - RNA, 30 nt, synthetic construct
  source 1..30: mol_type = other RNA
  modified_base 25: mod_base = i
  modified_base order(1,3,6..8,10,12,18,21,26,28..30): mod_base = OTHER; note = 2-prime-methoxyribose
  modified_base order(1,11,14,25,29): mod_base = OTHER; note = Mesyl phosphoroamidate linkage
  modified_base order(2,4..5,9,11,13..17,19..20,22,27): mod_base = OTHER; note = 2-prime-deoxy-2-prime-fluororibose
  modified_base order(23,25): mod_base = OTHER; note = deoxyribose
  modified_base 24: mod_base = OTHER; note = beta-homoDNA
  modified_base order(2..10,12..13,15..24,26..28): mod_base = OTHER; note = Phosphorothioate linkage
cataatttacacagaagcaatgccntcacc

SEQ ID NO: 6 - RNA, 30 nt, synthetic construct; identical to SEQ ID NO: 5
  source 1..30: mol_type = other RNA
  modified_base 25: mod_base = i
  modified_base order(1,3,6..8,10,12,18,21,26,28..30): mod_base = OTHER; note = 2-prime-methoxyribose
  modified_base order(1,11,14,25,29): mod_base = OTHER; note = Mesyl phosphoroamidate linkage
  modified_base order(2,4..5,9,11,13..17,19..20,22,27): mod_base = OTHER; note = 2-prime-deoxy-2-prime-fluororibose
  modified_base order(23,25): mod_base = OTHER; note = deoxyribose
  modified_base 24: mod_base = OTHER; note = beta-homoDNA
  modified_base order(2..10,12..13,15..24,26..28): mod_base = OTHER; note = Phosphorothioate linkage

SEQ ID NO: 7 - RNA, 30 nt, synthetic construct; identical to SEQ ID NO: 5
  source 1..30: mol_type = other RNA
  modified_base 25: mod_base = i
  modified_base order(1,3,6..8,10,12,18,21,26,28..30): mod_base = OTHER; note = 2-prime-methoxyribose
  modified_base order(1,11,14,25,29): mod_base = OTHER; note = Mesyl phosphoroamidate linkage
  modified_base order(2,4..5,9,11,13..17,19..20,22,27): mod_base = OTHER; note = 2-prime-deoxy-2-prime-fluororibose
  modified_base order(23,25): mod_base = OTHER; note = deoxyribose
  modified_base 24: mod_base = OTHER; note = beta-homoDNA
  modified_base order(2..10,12..13,15..24,26..28): mod_base = OTHER; note = Phosphorothioate linkage

SEQ ID NO: 8 - RNA, 30 nt, synthetic construct; identical to SEQ ID NO: 5
  source 1..30: mol_type = other RNA
  modified_base 25: mod_base = i
  modified_base order(1,3,6..8,10,12,18,21,26,28..30): mod_base = OTHER; note = 2-prime-methoxyribose
  modified_base order(1,11,14,25,29): mod_base = OTHER; note = Mesyl phosphoroamidate linkage
  modified_base order(2,4..5,9,11,13..17,19..20,22,27): mod_base = OTHER; note = 2-prime-deoxy-2-prime-fluororibose
  modified_base order(23,25): mod_base = OTHER; note = deoxyribose
  modified_base 24: mod_base = OTHER; note = beta-homoDNA
  modified_base order(2..10,12..13,15..24,26..28): mod_base = OTHER; note = Phosphorothioate linkage

SEQ ID NO: 9 - RNA, 32 nt, synthetic construct
  source 1..32: mol_type = other RNA
  modified_base 25: mod_base = i
  modified_base order(1,3,6..8,10,12,18,21,26,28..30): mod_base = OTHER; note = 2-prime-methoxyribose
  modified_base order(1,11,14,25,29): mod_base = OTHER; note = Mesyl phosphoroamidate linkage
  modified_base order(2,4..5,9,11,13..17,19..20,22,27): mod_base = OTHER; note = 2-prime-deoxy-2-prime-fluororibose
  modified_base order(23,25,31..32): mod_base = OTHER; note = deoxyribose
  modified_base 24: mod_base = OTHER; note = beta-homoDNA
  modified_base order(2..10,12..13,15..24,26..28): mod_base = OTHER; note = Phosphorothioate linkage
  modified_base order(30,31): mod_base = OTHER; note = Phosphate linkage
cataatttacacagaagcaatgccntcaccaa

SEQ ID NO: 10 - RNA, 32 nt, synthetic construct
  source 1..32: mol_type = other RNA
  modified_base 27: mod_base = i
  modified_base order(3,5,8..10,12,14,20,23,28,30..32): mod_base = OTHER; note = 2-prime-methoxyribose
  modified_base order(3,13,16,27,31): mod_base = OTHER; note = Mesyl phosphoroamidate linkage
  modified_base order(4,6..7,11,13,15..19,21..22,24,29): mod_base = OTHER; note = 2-prime-deoxy-2-prime-fluororibose
  modified_base order(1..2,25,27): mod_base = OTHER; note = deoxyribose
  modified_base 26: mod_base = OTHER; note = beta-homoDNA
  modified_base order(4..12,14..15,17..26,28..30): mod_base = OTHER; note = Phosphorothioate linkage
  modified_base 1..2: mod_base = OTHER; note = Phosphate linkage
aacataatttacacagaagcaatgccntcacc

SEQ ID NO: 11 - RNA, 30 nt, synthetic construct; identical to SEQ ID NO: 1
  source 1..30: mol_type = other RNA
  modified_base 25: mod_base = i
  modified_base order(1..3,12,14,16,26,28..30): mod_base = OTHER; note = 2-prime-methoxyribose
  modified_base order(1,11,14,25,29): mod_base = OTHER; note = Mesyl phosphoroamidate linkage
  modified_base order(4..5,8..9,11,13,15,17..19,22,27): mod_base = OTHER; note = 2-prime-deoxy-2-prime-fluororibose
  modified_base order(6..7,10,20..21): mod_base = OTHER; note = 2-prime-MOEribose
  modified_base order(23,25): mod_base = OTHER; note = deoxyribose
  modified_base 24: mod_base = OTHER; note = beta-homoDNA
  modified_base order(2..5,8..9,13,16..19,22..24,27..28): mod_base = OTHER; note = Phosphorothioate linkage
  modified_base order(6..7,10,12,15,20..21,26): mod_base = OTHER; note = Phosphate linkage
  modified_base order(10,20..21,23): mod_base = OTHER; note = Thymine

SEQ ID NO: 12 - RNA, 42 nt, synthetic construct
  source 1..42: mol_type = other RNA
  modified_base 33: mod_base = i
  modified_base order(3..4,9,11,13..14,17,19..21,24,28,30,34,36..39): mod_base = OTHER; note = 2-prime-methoxyribose
  modified_base order(1,21,23,33,41): mod_base = OTHER; note = Mesyl phosphoroamidate linkage
  modified_base order(5..8,10,12,15..16,18,22..23,25..27,29,35,40): mod_base = OTHER; note = 2-prime-deoxy-2-prime-fluororibose
  modified_base order(1..2,41..42): mod_base = OTHER; note = 2-prime-MOEribose
  modified_base 31: mod_base = OTHER; note = deoxyribose
  modified_base 32: mod_base = OTHER; note = beta-homoDNA
  modified_base order(2,4..12,14..18,22,24..32,34..35,39..40): mod_base = OTHER; note = Phosphorothioate linkage
  modified_base 42: mod_base = OTHER; note = 5-methyl-cytidine
  modified_base order(3,13,19..20,36..38): mod_base = OTHER; note = Phosphate linkage
  modified_base order(31,41): mod_base = OTHER; note = Thymine
  modified_base 33: mod_base = OTHER; note = 2-prime-fluroarabinose
aacatggccccagcagcttcagtccctttctcntcgatggtc

SEQ ID NO: 13 - RNA, 42 nt, synthetic construct; identical to SEQ ID NO: 12
  source 1..42: mol_type = other RNA
  modified_base 33: mod_base = i
  modified_base order(3..4,9,11,13..14,17,19..21,24,28,30,34,36..39): mod_base = OTHER; note = 2-prime-methoxyribose
  modified_base order(1,21,23,33,41): mod_base = OTHER; note = Mesyl phosphoroamidate linkage
  modified_base order(5..8,10,12,15..16,18,22..23,25..27,29,35,40): mod_base = OTHER; note = 2-prime-deoxy-2-prime-fluororibose
  modified_base order(1..2,41..42): mod_base = OTHER; note = 2'-MOEribose
  modified_base 31: mod_base = OTHER; note = deoxyribose
  modified_base 32: mod_base = OTHER; note = beta-homoDNA
  modified_base order(2,4..12,14..18,22,24..32,34..35,39..40): mod_base = OTHER; note = Phosphorothioate linkage
  modified_base 42: mod_base = OTHER; note = 5-methyl-cytidine
  modified_base order(3,13,19..20,36..38): mod_base = OTHER; note = Phosphate linkage
  modified_base order(31,41): mod_base = OTHER; note = Thymine
  modified_base 33: mod_base = OTHER; note = 2'-fluroarabinose

SEQ ID NO: 14 - RNA, 30 nt, synthetic construct
  source 1..30: mol_type = other RNA
  modified_base 26: mod_base = i
  modified_base order(1,5,9,13,17,21,27,29..30): mod_base = OTHER; note = 2-prime-methoxyribose
  modified_base order(1,14,16,26,29): mod_base = OTHER; note = Mesyl phosphoroamidate linkage
  modified_base order(2..4,6..8,10..12,14..16,18..20,22..24,28): mod_base = OTHER; note = 2-prime-deoxy-2-prime-fluororibose
  modified_base 26: mod_base = OTHER; note = deoxyribose
  modified_base 25: mod_base = OTHER; note = beta-homoDNA
  modified_base order(2..13,15,17..25,27..28): mod_base = OTHER; note = Phosphorothioate linkage
ccccagcagcttcagtccctttctcntcga

SEQ ID NO: 15 - RNA, 30 nt, synthetic construct; identical to SEQ ID NO: 14
  source 1..30: mol_type = other RNA
  modified_base 26: mod_base = i
  modified_base order(1,5,9,13,17,21,27,29..30): mod_base = OTHER; note = 2-prime-methoxyribose
  modified_base order(1,14,16,26,29): mod_base = OTHER; note = Mesyl phosphoroamidate linkage
  modified_base order(2..4,6..8,10..12,14..16,18..20,22..24,28): mod_base = OTHER; note = 2-prime-deoxy-2-prime-fluororibose
  modified_base 26: mod_base = OTHER; note = deoxyribose
  modified_base 25: mod_base = OTHER; note = beta-homoDNA
  modified_base order(2..13,15,17..25,27..28): mod_base = OTHER; note = Phosphorothioate linkage

SEQ ID NO: 16 - RNA, 50 nt, synthetic construct
  source 1..50: mol_type = other RNA
  modified_base order(1..4,6..10,12,14,16,19,21,28,32..33,35,39..50): mod_base = OTHER; note = 2-prime-methoxyribose
  modified_base order(5,11,13,15,17..18,20,22..27,29..31,34): mod_base = OTHER; note = 2-prime-deoxy-2-prime-fluororibose
  modified_base 36..38: mod_base = OTHER; note = deoxyribose
  modified_base order(1..5,7,9,11,13,15,17..18,20,22..27,29..32,34,36..38,46..49): mod_base = OTHER; note = Phosphorothioate linkage
  modified_base order(6,8,10,12,14,16,19,21,28,33,35,39..45): mod_base = OTHER; note = Phosphate linkage
cataattcttgtgtctactgtacagaatactgccgccagctggatttccc

SEQ ID NO: 17 - RNA, 50 nt, synthetic construct; identical to SEQ ID NO: 16
  source 1..50: mol_type = other RNA
  modified_base order(1..4,6..10,12,14,16,19,21,28,32..33,35,39..50): mod_base = OTHER; note = 2-prime-methoxyribose
  modified_base order(5,11,13,15,17..18,20,22..27,29..31,34): mod_base = OTHER; note = 2-prime-deoxy-2-prime-fluororibose
  modified_base 36..38: mod_base = OTHER; note = deoxyribose
  modified_base order(1..5,7,9,11,13,15,17..18,20,22..27,29..32,34,36..38,46..49): mod_base = OTHER; note = Phosphorothioate linkage
  modified_base order(6,8,10,12,14,16,19,21,28,33,35,39..45): mod_base = OTHER; note = Phosphate linkage

SEQ ID NO: 18 - RNA, 50 nt, synthetic construct; identical to SEQ ID NO: 16
  source 1..50: mol_type = other RNA
  modified_base order(1..4,6..10,12,14,16,19,21,28,32..33,35,39..50): mod_base = OTHER; note = 2-prime-methoxyribose
  modified_base order(5,11,13,15,17..18,20,22..27,29..31,34): mod_base = OTHER; note = 2-prime-deoxy-2-prime-fluororibose
  modified_base 36..38: mod_base = OTHER; note = deoxyribose
  modified_base order(1..5,7,9,11,13,15,17..18,20,22..27,29..32,34,36..38,46..49): mod_base = OTHER; note = Phosphorothioate linkage
  modified_base order(6,8,10,12,14,16,19,21,28,33,35,39..45): mod_base = OTHER; note = Phosphate linkage

SEQ ID NO: 19 - RNA, 73 nt, synthetic construct
  source 1..73: mol_type = other RNA
  modified_base order(1..3,5..6,20,23,25,27..28,32,34..35,37..41,43..49,52,54..61,65..73): mod_base = OTHER; note = 2-prime-methoxyribose
  modified_base order(4,7..19,21..22,24,26,29..31,33,36,42,50..51,53): mod_base = OTHER; note = 2-prime-deoxy-2-prime-fluororibose
  modified_base 62..64: mod_base = OTHER; note = deoxyribose
  modified_base order(1..2,4,7..19,21..22,24,26,29..31,33,36,42,50..51,53,62..64,69..72): mod_base = OTHER; note = Phosphorothioate linkage
  modified_base order(3,5..6,20,23,25,27..28,32,34..35,37..41,43..49,52,54..61,65..68): mod_base = OTHER; note = Phosphate linkage
ggtgtcgagaagaggagaacaatatgctaaatgttgttctcgtctcctcgacaccctgccgccagctggattt

SEQ ID NO: 20 - RNA, 73 nt, synthetic construct; identical to SEQ ID NO: 19
  source 1..73: mol_type = other RNA
  modified_base order(1..3,5..6,20,23,25,27..28,32,34..35,37..41,43..49,52,54..61,65..73): mod_base = OTHER; note = 2-prime-methoxyribose
  modified_base order(4,7..19,21..22,24,26,29..31,33,36,42,50..51,53): mod_base = OTHER; note = 2-prime-deoxy-2-prime-fluororibose
  modified_base 62..64: mod_base = OTHER; note = deoxyribose
  modified_base order(1..2,4,7..19,21..22,24,26,29..31,33,36,42,50..51,53,62..64,69..72): mod_base = OTHER; note = Phosphorothioate linkage
  modified_base order(3,5..6,20,23,25,27..28,32,34..35,37..41,43..49,52,54..61,65..68): mod_base = OTHER; note = Phosphate linkage

SEQ ID NO: 21 - RNA, 50 nt, synthetic construct; identical to SEQ ID NO: 16
  source 1..50: mol_type = other RNA
  modified_base order(1..4,6..10,12,14,16,19,21,28,32..33,35,39..50): mod_base = OTHER; note = 2-prime-methoxyribose
  modified_base order(5,11,13,15,17..18,20,22..27,29..31,34): mod_base = OTHER; note = 2-prime-deoxy-2-prime-fluororibose
  modified_base 36..38: mod_base = OTHER; note = deoxyribose
  modified_base order(1..5,7,9,11,13,15,17..18,20,22..27,29..32,34,36..38,46..49): mod_base = OTHER; note = Phosphorothioate linkage
  modified_base order(6,8,10,12,14,16,19,21,28,33,35,39..45): mod_base = OTHER; note = Phosphate linkage

SEQ ID NO: 22 - RNA, 50 nt, synthetic construct; identical to SEQ ID NO: 16
  source 1..50: mol_type = other RNA
  modified_base order(1..4,6..10,12,14,16,19,21,28,32..33,35,39..50): mod_base = OTHER; note = 2-prime-methoxyribose
  modified_base order(5,11,13,15,17..18,20,22..27,29..31,34): mod_base = OTHER; note = 2-prime-deoxy-2-prime-fluororibose
  modified_base order(1..5,7,9,11,13,15,17..18,20,22..27,29..32,34,36..38,46..49): mod_base = OTHER; note = Phosphorothioate linkage
  modified_base order(6,8,10,12,14,16,19,21,28,33,35,39..45): mod_base = OTHER; note = Phosphate linkage
  modified_base 36..38: mod_base = OTHER; note = deoxyribose

SEQ ID NO: 23 - RNA, 50 nt, synthetic construct; identical to SEQ ID NO: 16
  source 1..50: mol_type = other RNA
  modified_base order(1..4,6..10,12,14,16,19,21,28,32..33,35,39..50): mod_base = OTHER; note = 2-prime-methoxyribose
  modified_base order(5,11,13,15,17..18,20,22..27,29..31,34): mod_base = OTHER; note = 2-prime-deoxy-2-prime-fluororibose
  modified_base 36..38: mod_base = OTHER; note = deoxyribose
  modified_base order(1..5,7,9,11,13,15,17..18,20,22..27,29..32,34,36..38,46..49): mod_base = OTHER; note = Phosphorothioate linkage
  modified_base order(6,8,10,12,14,16,19,21,28,33,35,39..45): mod_base = OTHER; note = Phosphate linkage

SEQ ID NO: 24 - RNA, 52 nt, synthetic construct
  source 1..52: mol_type = other RNA
  modified_base order(1..4,6..10,12,14,16,19,21,28,32..33,35,39..50): mod_base = OTHER; note = 2-prime-methoxyribose
  modified_base order(5,11,13,15,17..18,20,22..27,29..31,34): mod_base = OTHER; note = 2-prime-deoxy-2-prime-fluororibose
  modified_base order(36..38,51..52): mod_base = OTHER; note = deoxyribose
  modified_base order(1..5,7,9,11,13,15,17..18,20,22..27,29..32,34,36..38,46..49): mod_base = OTHER; note = Phosphorothioate linkage
  modified_base order(6,8,10,12,14,16,19,21,28,33,35,39..45,50..51): mod_base = OTHER; note = Phosphate linkage
cataattcttgtgtctactgtacagaatactgccgccagctggatttcccaa

SEQ ID NO: 25 - RNA, 52 nt, synthetic construct
  source 1..52: mol_type = other RNA
  modified_base order(3..6,8..12,14,16,18,21,23,30,34..35,37,41..52): mod_base = OTHER; note = 2-prime-methoxyribose
  modified_base order(7,13,15,17,19..20,22,24..29,31..33,36): mod_base = OTHER; note = 2-prime-deoxy-2-prime-fluororibose
  modified_base order(1..2,38..40): mod_base = OTHER; note = deoxyribose
  modified_base order(3..7,9,11,13,15,17,19..20,22,24..29,31..34,36,38..40,48..51): mod_base = OTHER; note = Phosphorothioate linkage
  modified_base order(1..2,8,10,12,14,16,18,21,23,30,35,37,41..47): mod_base = OTHER; note = Phosphate linkage
aacataattcttgtgtctactgtacagaatactgccgccagctggatttccc

SEQ ID NO: 26 - RNA, 53 nt, synthetic construct
  source 1..53: mol_type = other RNA
  modified_base order(1..4,6..7,9..11,14..17,19,22..23,25,29,31..32,35,37..40,43..45,47..48,50..53): mod_base = OTHER; note = 2-prime-methoxyribose
  modified_base order(5,8,12..13,18,20..21,24,30,33..34,36,41..42,46,49): mod_base = OTHER; note = 2-prime-deoxy-2-prime-fluororibose
  modified_base 26..28: mod_base = OTHER; note = deoxyribose
  modified_base order(1..6,8,10,12..14,16,18,20..22,24,26..28,30,32..34,36,38,40..42,44,46,48..52): mod_base = OTHER; note = Phosphorothioate linkage
  modified_base order(7,9,11,15,17,19,23,25,29,31,35,37,39,43,45,47): mod_base = OTHER; note = Phosphate linkage
gtgtctactgtacagcatactgccgccagctggatttgccaattctgagtaac

SEQ ID NO: 27 - RNA, 53 nt, synthetic construct; identical to SEQ ID NO: 26
  source 1..53: mol_type = other RNA
  modified_base order(1..4,6..7,9..11,14..17,19,22..23,25,29,31..32,35,37..40,43..45,47..48,50..53): mod_base = OTHER; note = 2-prime-methoxyribose
  modified_base order(5,8,12..13,18,20..21,24,30,33..34,36,41..42,46,49): mod_base = OTHER; note = 2-prime-deoxy-2-prime-fluororibose
  modified_base 26..28: mod_base = OTHER; note = deoxyribose
  modified_base order(1..6,8,10,12..14,16,18,20..22,24,26..28,30,32..34,36,38,40..42,44,46,48..52): mod_base = OTHER; note = Phosphorothioate linkage
  modified_base order(7,9,11,15,17,19,23,25,29,31,35,37,39,43,45,47): mod_base = OTHER; note = Phosphate linkage

SEQ ID NO: 28 - RNA, 53 nt, synthetic construct; identical to SEQ ID NO: 26
  source 1..53: mol_type = other RNA
  modified_base order(1..4,6..7,9..11,14..17,19,22..23,25,29,31..32,35,37..40,43..45,47..48,50..53): mod_base = OTHER; note = 2-prime-methoxyribose
  modified_base order(5,8,12..13,18,20..21,24,30,33..34,36,41..42,46,49): mod_base = OTHER; note = 2-prime-deoxy-2-prime-fluororibose
  modified_base order(1..6,8,10,12..14,16,18,20..22,24,26..28,30,32..34,36,38,40..42,44,46,48..52): mod_base = OTHER; note = Phosphorothioate linkage
  modified_base order(7,9,11,15,17,19,23,25,29,31,35,37,39,43,45,47): mod_base = OTHER; note = Phosphate linkage
  modified_base 26..28: mod_base = OTHER; note = deoxyribose

SEQ ID NO: 29 - RNA, 53 nt, synthetic construct; identical to SEQ ID NO: 26
  source 1..53: mol_type = other RNA
  modified_base order(1..4,6..7,9..11,14..17,19,22..23,25,29,31..32,35,37..40,43..45,47..48,50..53): mod_base = OTHER; note = 2-prime-methoxyribose
  modified_base order(5,8,12..13,18,20..21,24,30,33..34,36,41..42,46,49): mod_base = OTHER; note = 2-prime-deoxy-2-prime-fluororibose
  modified_base 26..28: mod_base = OTHER; note = deoxyribose
  modified_base order(1..6,8,10,12..14,16,18,20..22,24,26..28,30,32..34,36,38,40..42,44,46,48..52): mod_base = OTHER; note = Phosphorothioate linkage
  modified_base order(7,9,11,15,17,19,23,25,29,31,35,37,39,43,45,47): mod_base = OTHER; note = Phosphate linkage

SEQ ID NO: 30 - RNA, 53 nt, synthetic construct; identical to SEQ ID NO: 26
  source 1..53: mol_type = other RNA
  modified_base order(1..4,6..7,9..11,14..17,19,22..23,25,29,31..32,35,37..40,43..45,47..48,50..53): mod_base = OTHER; note = 2-prime-methoxyribose
  modified_base order(5,8,12..13,18,20..21,24,30,33..34,36,41..42,46,49): mod_base = OTHER; note = 2-prime-deoxy-2-prime-fluororibose
  modified_base 26..28: mod_base = OTHER; note = deoxyribose
  modified_base order(1..6,8,10,12..14,16,18,20..22,24,26..28,30,32..34,36,38,40..42,44,46,48..52): mod_base = OTHER; note = Phosphorothioate linkage
  modified_base order(7,9,11,15,17,19,23,25,29,31,35,37,39,43,45,47): mod_base = OTHER; note = Phosphate linkage

SEQ ID NO: 31 - RNA, 53 nt, synthetic construct; identical to SEQ ID NO: 26
  source 1..53: mol_type = other RNA
  modified_base order(1..4,6..7,9..11,14..17,19,22..23,25,29,31..32,35,37..40,43..45,47..48,50..53): mod_base = OTHER; note = 2-prime-methoxyribose
  modified_base order(5,8,12..13,18,20..21,24,30,33..34,36,41..42,46,49): mod_base = OTHER; note = 2-prime-deoxy-2-prime-fluororibose
  modified_base order(1..6,8,10,12..14,16,18,20..22,24,26..28,30,32..34,36,38,40..42,44,46,48..52): mod_base = OTHER; note = Phosphorothioate linkage
  modified_base order(7,9,11,15,17,19,23,25,29,31,35,37,39,43,45,47): mod_base = OTHER; note = Phosphate linkage
  modified_base 26..28: mod_base = OTHER; note = deoxyribose

SEQ ID NO: 32 - RNA, 30 nt, synthetic construct; identical to SEQ ID NO: 1
  source 1..30: mol_type = other RNA
  modified_base 25: mod_base = i
  modified_base order(6..7,10..14,20,26,28,30): mod_base = OTHER; note = 2-prime-methoxyribose
  modified_base order(1,3,8,15,25,29): mod_base = OTHER; note = Mesyl phosphoroamidate linkage
  modified_base order(4..5,8..9,15,17,19,22,27,29): mod_base = OTHER; note = 2-prime-deoxy-2-prime-fluororibose
  modified_base order(1..3,16,18,21): mod_base = OTHER; note = 2-prime-MOEribose
  modified_base order(23,25): mod_base = OTHER; note = deoxyribose
  modified_base 24: mod_base = OTHER; note = beta-homoDNA
  modified_base order(2,4..5,9,17,19,22..24,27..28): mod_base = OTHER; note = Phosphorothioate linkage
  modified_base order(1..3,16,18): mod_base = OTHER; note = 5-methyl-cytidine
  modified_base order(6..7,10..14,16,18,20..21,26): mod_base = OTHER; note = Phosphate linkage
  modified_base order(21,23): mod_base = OTHER; note = Thymine

SEQ ID NO: 33 - RNA, 30 nt, synthetic construct; identical to SEQ ID NO: 1
  source 1..30: mol_type = other RNA
  modified_base 25: mod_base = i
  modified_base order(1,6..7,12..14,18,20,26,28,30): mod_base = OTHER; note = 2-prime-methoxyribose
  modified_base order(1,3,8,15,25,29): mod_base = OTHER; note = Mesyl phosphoroamidate linkage
  modified_base order(4..5,8..9,15,17,19,22,27,29): mod_base = OTHER; note = 2-prime-deoxy-2-prime-fluororibose
  modified_base order(2..3,10..11,16,21): mod_base = OTHER; note = 2-prime-MOEribose
  modified_base order(23,25): mod_base = OTHER; note = deoxyribose
  modified_base 24: mod_base = OTHER; note = beta-homoDNA
  modified_base order(2,4..5,9,17,19,22..24,27..28): mod_base = OTHER; note = Phosphorothioate linkage
  modified_base order(2..3,16): mod_base = OTHER; note = 5-methyl-cytidine
  modified_base order(6..7,10..14,16,18,20..21,26): mod_base = OTHER; note = Phosphate linkage
  modified_base order(10..11,21,23): mod_base = OTHER; note = Thymine

SEQ ID NO: 34 - RNA, 30 nt, synthetic construct; identical to SEQ ID NO: 1
  source 1..30: mol_type = other RNA
  modified_base 25: mod_base = i
  modified_base order(6..7,10..14,20,26,28,30): mod_base = OTHER; note = 2-prime-methoxyribose
  modified_base order(1,3,8,15,25,29): mod_base = OTHER; note = Mesyl phosphoroamidate linkage
  modified_base order(4..5,8..9,15,17,19,22,27,29): mod_base = OTHER; note = 2-prime-deoxy-2-prime-fluororibose
  modified_base order(1..3,16,18,21): mod_base = OTHER; note = 2-prime-MOEribose
  modified_base order(23,25): mod_base = OTHER; note = deoxyribose
  modified_base 24: mod_base = OTHER; note = beta-homoDNA
  modified_base order(2,4..5,9,17,19,22..24,27..28): mod_base = OTHER; note = Phosphorothioate linkage
  modified_base order(1..3,16,18): mod_base = OTHER; note = 5-methyl-cytidine
  modified_base order(6..7,10..14,16,18,20..21,26): mod_base = OTHER; note = Phosphate linkage
  modified_base order(21,23): mod_base = OTHER; note = Thymine

SEQ ID NO: 35 - RNA, 29 nt, synthetic construct
  source 1..29: mol_type = other RNA
  modified_base 24: mod_base = i
  modified_base order(9,19..20,22): mod_base = OTHER; note = Thymine
cccagagcttcagtccctttctcntcgat

SEQ ID NO: 36 - RNA, 29 nt, synthetic construct
  source 1..29: mol_type = other RNA
  modified_base 24: mod_base = i
  modified_base order(9,19..20,22): mod_base = OTHER; note = Thymine
cccagagcttcagtccctttctcntcgat

SEQ ID NO: 37 - RNA, 29 nt, synthetic construct
  source 1..29: mol_type = other RNA
  modified_base 24: mod_base = i
  modified_base order(9,19..20,22): mod_base = OTHER; note = Thymine
cccagagcttcagtccctttctcntcgat

SEQ ID NO: 38 - RNA, 29 nt, synthetic construct
  source 1..29: mol_type = other RNA
  modified_base 24: mod_base = i
  modified_base order(9,10..20,22): mod_base = OTHER; note = Thymine
cccagagcttcagtccctttctcntcgat

SEQ ID NO: 39 - RNA, 30 nt, synthetic construct; identical to SEQ ID NO: 5
  source 1..30: mol_type = other RNA
  modified_base 25: mod_base = i

SEQ ID NO: 40 - RNA, 30 nt, synthetic construct; identical to SEQ ID NO: 5
  source 1..30: mol_type = other RNA
  modified_base 25: mod_base = i

SEQ ID NO: 41 - RNA, 30 nt, synthetic construct; identical to SEQ ID NO: 5
  source 1..30: mol_type = other RNA
  modified_base 25: mod_base = i

SEQ ID NO: 42 - RNA, 30 nt, synthetic construct; identical to SEQ ID NO: 5
  source 1..30: mol_type = other RNA
  modified_base 25: mod_base = i

SEQ ID NO: 43 - RNA, 32 nt, synthetic construct; identical to SEQ ID NO: 9
  source 1..32: mol_type = other RNA
  modified_base 25: mod_base = i

SEQ ID NO: 44 - RNA, 32 nt, synthetic construct; identical to SEQ ID NO: 10
  source 1..32: mol_type = other RNA
  modified_base 27: mod_base = i

SEQ ID NO: 45 - RNA, 29 nt, synthetic construct
  source 1..29: mol_type = other RNA
  modified_base 24: mod_base = i
  modified_base order(9,19..20,22): mod_base = OTHER; note = Thymine
cccagagcttcagtccctttctcntcgat

SEQ ID NO: 46 - RNA, 41 nt, synthetic construct
  source 1..41: mol_type = other RNA
  modified_base 33: mod_base = i
  modified_base order(31,41): mod_base = OTHER; note = Thymine
aacatggccccagcagcttcagtccctttctcntcgatggt

SEQ ID NO: 47 - RNA, 41 nt, synthetic construct; identical to SEQ ID NO: 46
  source 1..41: mol_type = other RNA
  modified_base 33: mod_base = i
  modified_base order(31,41): mod_base = OTHER; note = Thymine

SEQ ID NO: 48 - RNA, 30 nt, synthetic construct; identical to SEQ ID NO: 14
  source 1..30: mol_type = other RNA
  modified_base 26: mod_base = i

SEQ ID NO: 49 - RNA, 30 nt, synthetic construct; identical to SEQ ID NO: 14
  source 1..30: mol_type = other RNA
  modified_base 26: mod_base = i

SEQ ID NO: 50 - RNA, 50 nt, synthetic construct; identical to SEQ ID NO: 16
  source 1..50: mol_type = other RNA

SEQ ID NO: 51 - RNA, 50 nt, synthetic construct; identical to SEQ ID NO: 16
  source 1..50: mol_type = other RNA

SEQ ID NO: 52 - RNA, 50 nt, synthetic construct; identical to SEQ ID NO: 16
  source 1..50: mol_type = other RNA

SEQ ID NO: 53 - RNA, 73 nt, synthetic construct; identical to SEQ ID NO: 19
  source 1..73: mol_type = other RNA

SEQ ID NO: 54 - RNA, 73 nt, synthetic construct; identical to SEQ ID NO: 19
  source 1..73: mol_type = other RNA

SEQ ID NO: 55 - RNA, 50 nt, synthetic construct; identical to SEQ ID NO: 16
  source 1..50: mol_type = other RNA

SEQ ID NO: 56 - RNA, 50 nt, synthetic construct; identical to SEQ ID NO: 16
  source 1..50: mol_type = other RNA

SEQ ID NO: 57 - RNA, 50 nt, synthetic construct; identical to SEQ ID NO: 16
  source 1..50: mol_type = other RNA

SEQ ID NO: 58 - RNA, 52 nt, synthetic construct; identical to SEQ ID NO: 24
  source 1..52: mol_type = other RNA

SEQ ID NO: 59 - RNA, 52 nt, synthetic construct; identical to SEQ ID NO: 25
  source 1..52: mol_type = other RNA

SEQ ID NO: 60 - RNA, 53 nt, synthetic construct; identical to SEQ ID NO: 26
  source 1..53: mol_type = other RNA

SEQ ID NO: 61 - RNA, 53 nt, synthetic construct; identical to SEQ ID NO: 26
  source 1..53: mol_type = other RNA

SEQ ID NO: 62 - RNA, 53 nt, synthetic construct; identical to SEQ ID NO: 26
  source 1..53: mol_type = other RNA

SEQ ID NO: 63 - RNA, 53 nt, synthetic construct; identical to SEQ ID NO: 26
  source 1..53: mol_type = other RNA

SEQ ID NO: 64 - RNA, 53 nt, synthetic construct; identical to SEQ ID NO: 26
  source 1..53: mol_type = other RNA

SEQ ID NO: 65 - RNA, 53 nt, synthetic construct; identical to SEQ ID NO: 26
  source 1..53: mol_type = other RNA

SEQ ID NO: 66 - RNA, 25 nt, synthetic construct
  source 1..25: mol_type = other RNA
  modified_base 20: mod_base = i
  modified_base order(16,18): mod_base = OTHER; note = Thymine
agcagcttcagtctttctcntcgat

SEQ ID NO: 67 - RNA, 27 nt, synthetic construct
  source 1..27: mol_type = other RNA
  modified_base 22: mod_base = i
  modified_base order(8..9,18,20): mod_base = OTHER; note = Thymine
cagcagcttcagtcctttctcntcgat

SEQ ID NO: 68 - RNA, 25 nt, synthetic construct
  source 1..25: mol_type = other RNA
  modified_base 20: mod_base = i
  modified_base order(16,18): mod_base = OTHER; note = Thymine
agcagcttcagtctttctcntcgat

SEQ ID NO: 69 - DNA, 24 bp, Mus musculus
  source 1..24: mol_type = genomic DNA
taagtggttacaggaagtccctca

SEQ ID NO: 70 - DNA, 21 bp, Mus musculus
  source 1..21: mol_type = genomic DNA
ggaggcctcagacctgggcca

SEQ ID NO: 71 - DNA, 24 bp, Mus musculus
  source 1..24: mol_type = genomic DNA
taagtggttacaggaagtccctca

SEQ ID NO: 72 - DNA, 21 bp, Mus musculus
  source 1..21: mol_type = genomic DNA
ggaggcctcagacctgggcca

SEQ ID NO: 73 - DNA, 23 bp, synthetic construct
  source 1..23: mol_type = other DNA
acctatgatctgaagagcgtcct

SEQ ID NO: 74 - DNA, 25 bp, synthetic construct
  source 1..25: mol_type = other DNA
ttcaatcattaagaagacaaagggt

SEQ ID NO: 75 - DNA, 25 bp, Mus musculus
  source 1..25: mol_type = genomic DNA
gaattcctggattgtgtgtttaagt

SEQ ID NO: 76 - DNA, 24 bp, Mus musculus
  source 1..24: mol_type = genomic DNA
cagaagggaactgtctagttcagt

(19) United States
(12) Patent Application Publication
(10) Pub. No.: US 2026/0071212 A1
(43) Pub. Date: Mar. 12, 2026

(54) GalNAc conjugated oligonucleotides for RNA editing

(71) Applicant: KORRO BIO, INC., Cambridge, MA (US)

(72) Inventors: Venkat Krishnamurthy, Holliston, MA (US); Tyson Moyer, Cambridge, MA (US); Christopher Brown, Ashland, MA (US); Praveen Kumar Pogula, Cambridge, MA (US); Ayman Alawneh, Cambridge, MA (US)

(21) Appl. No.: 19/329,194

(22) Filed: Sep. 15, 2025

Related U.S. Application Data
(63) Continuation of application No. PCT/US25/43523, filed on Aug. 26, 2025.
(60) Provisional application No. 63/687,147, filed on Aug. 26, 2024.

(51) Int. Cl. C12N 15/11

(52) U.S. Cl. CPC C12N 15/11; C12N 2310/314; C12N 2310/315; C12N 2310/351

(57) ABSTRACT

Provided herein are modified oligonucleotides comprising novel GalNAc-containing moieties. Also provided are formulations comprising the modified oligonucleotides disclosed herein, and methods of treating diseases and disorders using the modified oligonucleotides and formulations thereof disclosed herein.

### CROSS-REFERENCE TO RELATED APPLICATIONS

This is a continuation of International Application No. PCT/US25/43523, filed Aug. 26, 2025, which claims priority to U.S. Ser. No. 63/687,147, filed Aug. 26, 2024, the respective disclosures of which are each incorporated herein by reference in their entireties.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: 50011_SeqListing.xml; Size: 220,730 bytes; Created: Aug. 25, 2025.

### BACKGROUND

The present disclosure generally relates to oligonucleotides conjugated to GalNAc moieties, formulations thereof, and methods of making and using the GalNAc-modified oligonucleotides and formulations thereof.

Several drugs derived from nucleic acids have been approved for commercialization in recent years, including fomivirsen, patisiran, and givosiran, and many more are in clinical trials. The liver, and especially hepatocytes, are attractive targets for RNA therapies (e.g., antisense oligonucleotide (ASO) and siRNA delivery) given their ability to be accessed directly by nanoparticle-sized constructs after simple intravascular injection. A drawback to this approach, however, is that RNA complexes are only passively targeted to liver (Rozema, D. B. et al., Applied Biological Sciences 2007, 104 (32) 12982-12987).

The asialoglycoprotein receptor (ASGPR), also known as hepatic binding protein or the Ashwell-Morell receptor, is a lectin occurring on the surface of hepatocytes. N-acetylgalactosamine, also known as GalNAc, is an aminosugar moiety that can recognize and bind to ASGPR. GalNAc modification of RNA has emerged as an attractive strategy for targeting hepatocytes with therapeutic nucleic acids; however, there remain challenges with delivery and stability of GalNAc-RNA conjugates (Springer, A. D. et al., Nucleic Acid Ther. 2018 Jun. 1; 28(3): 109-118).

Accordingly, there is a need for GalNAc-modified oligonucleotide therapeutics having improved properties, such as improved efficacy and stability.

### SUMMARY

Provided herein are modified oligonucleotides having a structure of Formula (I), (II), (III), (IV), or (V):

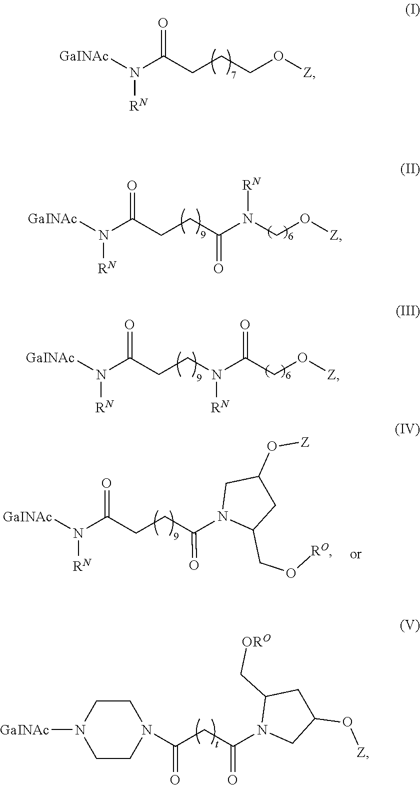

wherein GalNAc has a structure of

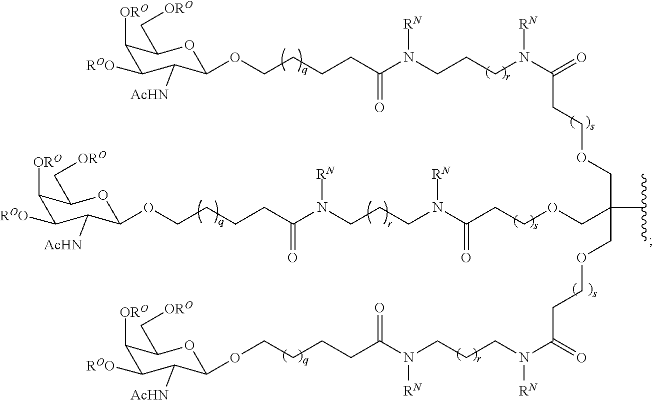

each R^{o} is independently H or C₁₋₃alkyl; each R^{N} is independently H or C₁₋₃alkyl; each q, r, and s is independently 0, 1, or 2; t is an integer from 1 to 20; and Z is an oligonucleotide moiety. In various embodiments, GalNAc has a structure of

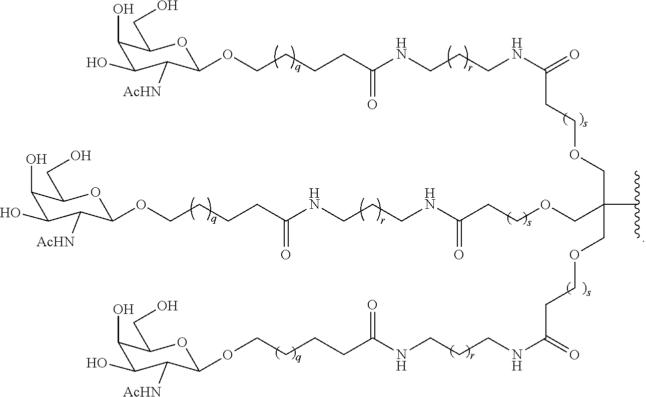

In various embodiments, provided herein are modified oligonucleotides having a structure of Formula (IA):

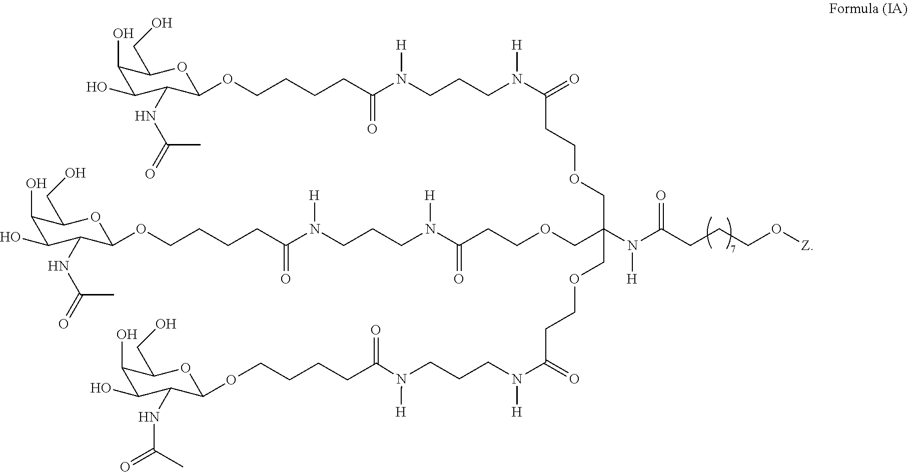

In various embodiments, provided herein are modified oligonucleotides having a structure of Formula (IIA):

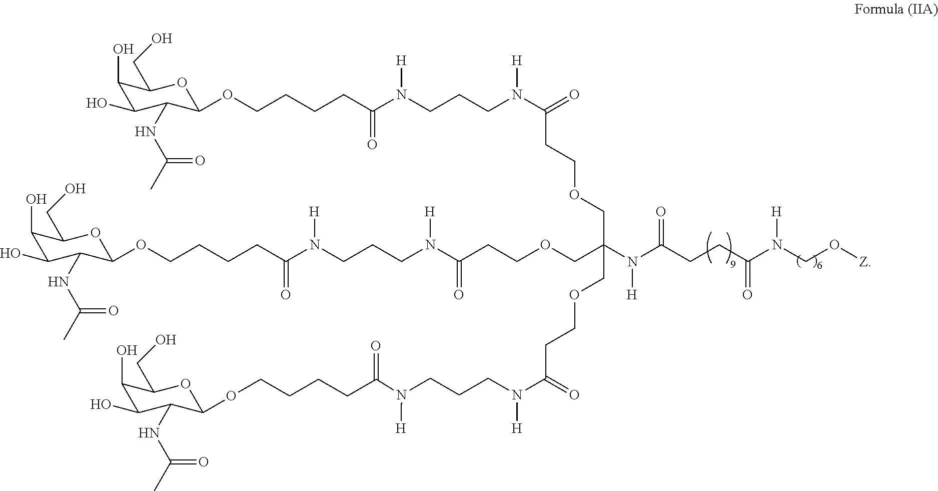

In various embodiments, provided herein are modified oligonucleotides having a structure of Formula (IIIA):

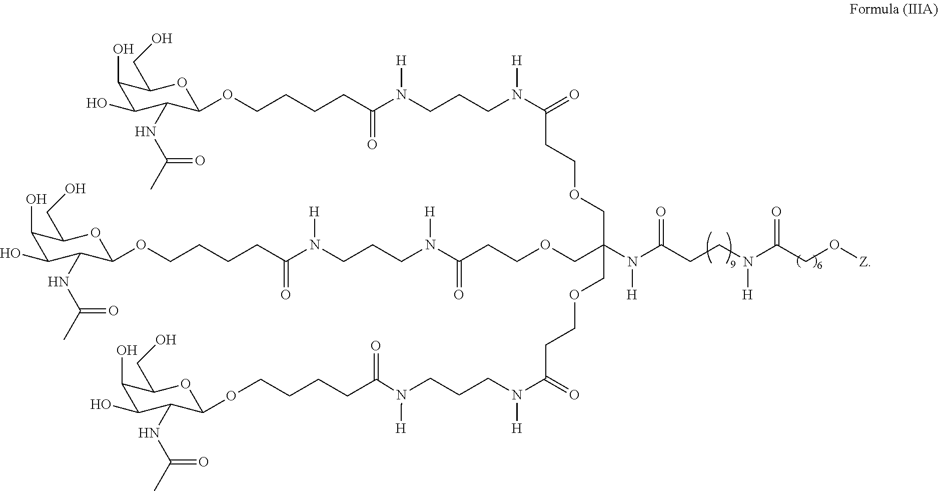

In various embodiments, provided herein are modified oligonucleotides having a structure of Formula (IVA):

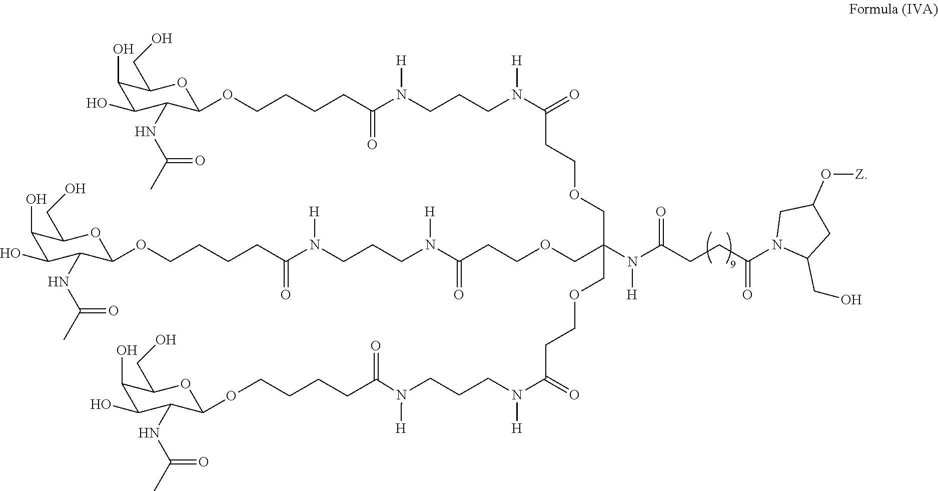

In various embodiments, provided herein are modified oligonucleotides having a structure of Formula VA:

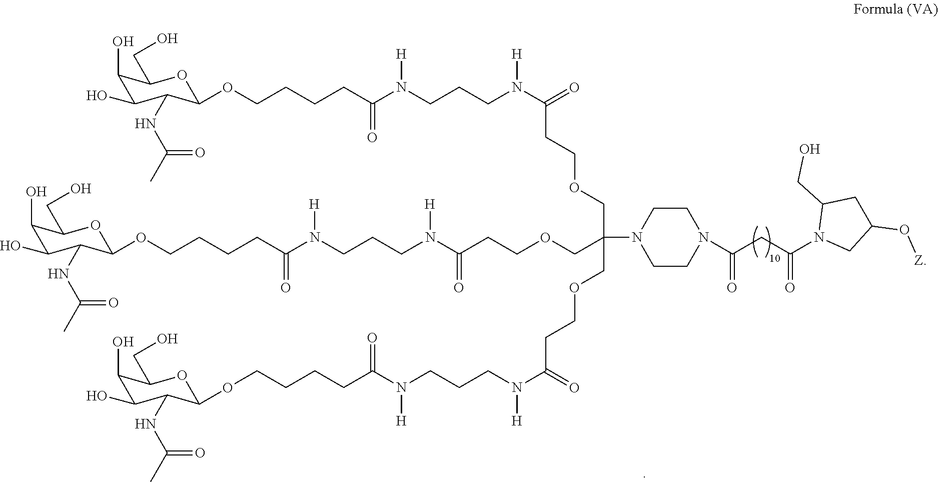

Also provided are formulations comprising the modified oligonucleotides disclosed herein. Further provided are methods of editing a target adenosine in a target RNA in a cell comprising contacting the cell with the modified oligonucleotides or the formulations thereof disclosed herein to (i) form a complex between the modified oligonucleotide and the target RNA; and (ii) recruit an ADAR in the cell to the complex such that the ADAR edits the target adenosine. Further provided are methods of treating a disease or disorder in a patient in need thereof, comprising administering to the patient a pharmaceutically effective amount of the modified oligonucleotides disclosed herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 shows the results of a study of in vitro editing of ACTB by GalNAc oligonucleotides described herein through transfection (top) or free uptake (bottom).

FIG. 2 shows the results of a study of in vivo editing of ACTB by GalNAc oligonucleotides described herein in wild-type (WT) mice.

FIG. 3 shows the results of a study of in vivo editing of ACTB by GalNAc oligonucleotides described herein in the livers of wild-type (WT) mice.

FIG. 4 shows the results of a study of in vitro editing of E342K by GalNAc oligonucleotides described herein through transfection (top) or free uptake (bottom).

FIG. 5 shows the results of a study of in vivo editing of E342K by GalNAc oligonucleotides described herein.

FIG. 6 shows the results of a study of in vitro editing of E342K by GalNAc oligonucleotides described herein as measured by FU (top) and Tfx (bottom) in PiZ mouse hepatocytes.

FIG. 7 shows the results of a study of in vivo editing of E342K by GalNAc oligonucleotides described herein in PiZ mice.

FIG. 8 shows the results of a study of in vitro editing of E342K by GalNAc oligonucleotides described herein through transfection (top) or free uptake (bottom).

FIG. 9 shows the results of a study of in vivo editing of E342K by GalNAc oligonucleotides described herein in PiZ mice.

FIG. 10 shows the results of a study of in vivo editing of Rab7a by GalNAc oligonucleotides described herein in the liver tissue of WT (top) and PiZ (bottom) mice.

FIG. 11 shows that the results of a study of in vivo editing of Rab7a by GalNAc oligonucleotides described herein in the liver tissue of WT and PiZ mice are reproducible across oligonucleotide formulations.

FIG. 12 shows the results of a study of in vitro editing of Rab7a by varying concentrations of GalNAc oligonucleotides described herein as measured in PiZ mouse hepatocytes. The study was run twice (N=1, top; and N=2, bottom). For each oligonucleotide tested, concentrations were (left to right) 0.037 nM, 0.185 nM, 0.93 nM, 4.63 nM, 13.8 nM, 41.67 nM, 125 nM, 250 nM, 500 nM, and 1000 nM.

FIG. 13 shows the percent editing by concentration of oligonucleotides disclosed herein, and EC50 data calculated from the curves.

FIG. 14 shows the results of a study of in vivo editing of Rab7a by GalNAc oligonucleotides described herein in mice.

### DETAILED DESCRIPTION

Provided herein are modified oligonucleotides comprising N-acetylgalactosamine (GalNAc oligonucleotides). Also provided are formulations comprising modified oligonucleotides, e.g., GalNAc oligonucleotides. Also provided are therapeutic methods using the modified oligonucleotides, e.g., GalNAc oligonucleotides, and the formulations thereof, and methods of making the modified oligonucleotides, e.g., GalNAc oligonucleotides, and the formulations thereof.

### GalNAc-Modified Oligonucleotides

Provided herein are modified oligonucleotides, e.g., oligonucleotides having at least one N-acetylgalactosamine, sometimes referred to as GalNAc oligonucleotides.

The oligonucleotides disclosed herein include modified oligonucleotides having a structure of Formula (I), (II), (III), (IV), or (V):

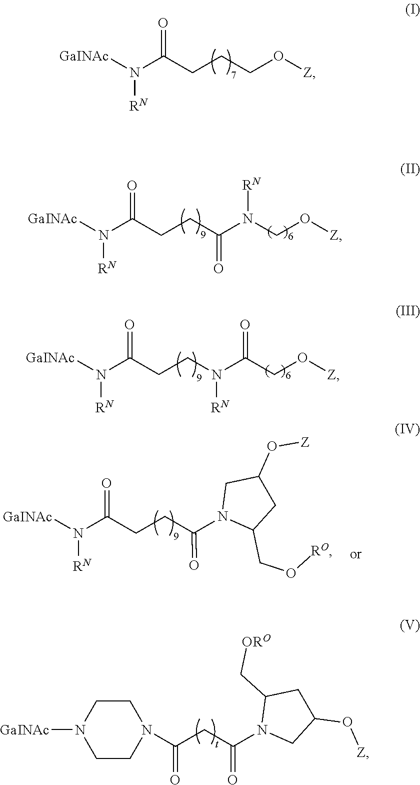

wherein
GalNAc has a structure of

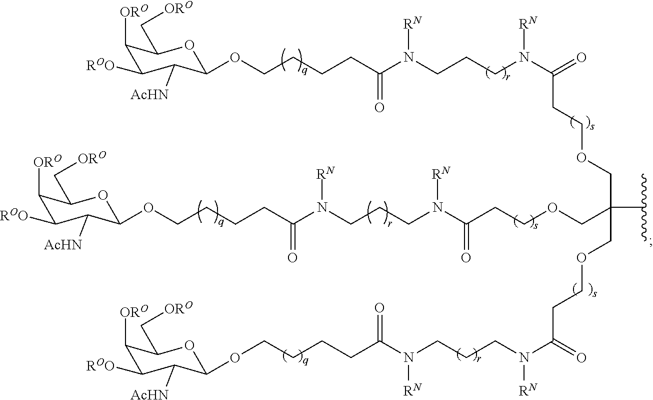

each R^{o} is independently H or C₁₋₃alkyl;
each R^{N} is independently H or C₁₋₃alkyl;
each q, r, and s is independently 0, 1, or 2;
t is an integer from 1 to 20; and
Z is an oligonucleotide moiety.

As used herein, the term “alkyl” or “alkylene” means a saturated straight or branched chain hydrocarbon. The term Cₙ means the alkyl group has “n” carbon atoms. For example, C₃alkyl refers to an alkyl group that has 3 carbon atoms. C₁₋₃alkyl refers to an alkyl group having a number of carbon atoms encompassing the entire range (i.e., 1 to 3 carbon atoms), as well as all subgroups (e.g., 1-3, 2-3, 1-2, 1, 2, and 3 carbon atoms). Specific examples include, but are not limited to, methyl, ethyl, isopropyl, and n-propyl.

In some cases, the modified oligonucleotides have the structure of Formula (I), (II), (IV), or (V). In some cases, the modified oligonucleotides have the structure of Formula (I), (III), (IV), or (V). In some cases, the modified oligonucleotides have the structure of Formula (I). In some cases, the modified oligonucleotides have the structure of Formula (II) or Formula (III). In some cases, the modified oligonucleotides have the structure of Formula (II). In some cases, the modified oligonucleotides have the structure of Formula (III). In some cases, the modified oligonucleotides have the structure of Formula (IV). In some cases, the modified oligonucleotides have the structure of Formula (V).

Modified oligonucleotides having the structure of Formula (I) are also referred to herein as “decanol” or “GD” oligonucleotides. Modified oligonucleotides having the structures of Formula (II) and Formula (III) are also referred to herein as “extended” or “GE” oligonucleotides. However, unless otherwise specified, the term “GE” used herein without further definition refers to oligonucleotides of Formula (II). Modified oligonucleotides having the structure of Formula (IV) are also referred to herein as “prolinol” or “GP” oligonucleotides.

For the compounds as disclosed herein (compounds for Formulae (I), (II), (III), (IV), and (V)), each R^{o} is independently H or C₁₋₃alkyl (e.g., methyl). In some cases, at least one R^{o} is H. In some cases, each R^{o} is H. In some cases, at least one R^{o} is C₁₋₃alkyl. In some cases, each R^{o} is C₁₋₃alkyl.

For the compounds as disclosed herein (compounds for Formulae (I), (II), (III), (IV) and (V)), each R^{N} is independently H or C₁₋₃alkyl (e.g., methyl). In some cases, at least one R^{N} is H. In some cases, each R^{N} is H. In some cases, at least one R^{N} is C₁₋₃alkyl. In some cases, each R^{N} is C₁₋₃alkyl.

For the compounds as disclosed herein (compounds for Formulae (I), (II), (III), (IV) and (V)), each q, r, and s is independently 0, 1, or 2. In some cases, at least one of q, r, and s is 1. In some cases, each q is 1. In some cases, each r is 1. In some cases, each s is 1. In some cases, each q, r, and s is independently 1.

In some cases, GalNAc has a structure of

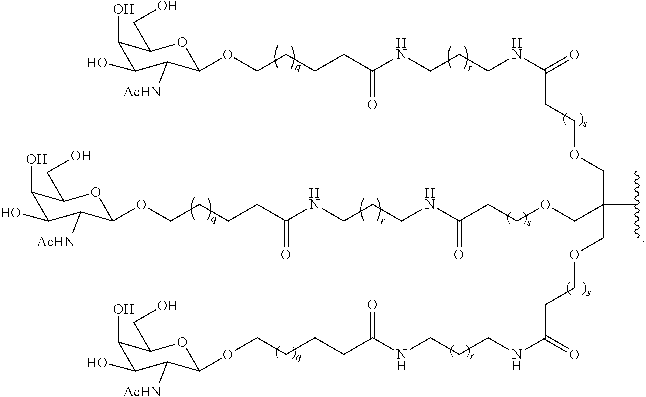

In some cases, GalNAc has a structure of

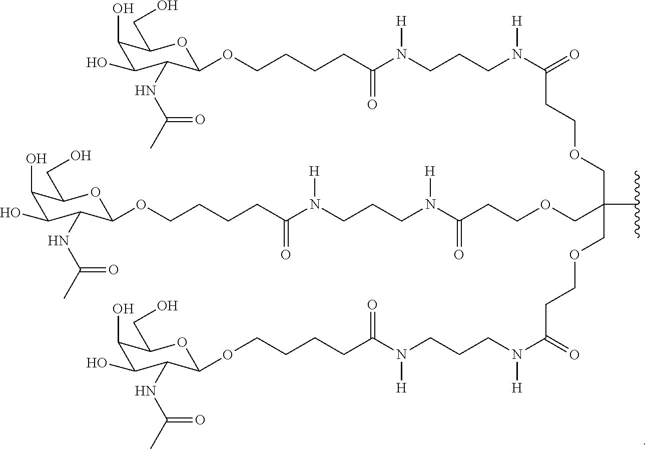 [SMILES: *C(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

For the compounds of Formula (V) as disclosed herein, t is an integer from 1 to 20. In some cases, t is 1 to 10. In some cases, t is 10 to 20. In some cases, t is 5 to 15. In some cases, t is 10.

In some cases, the modified oligonucleotide has a structure of Formula (IA):

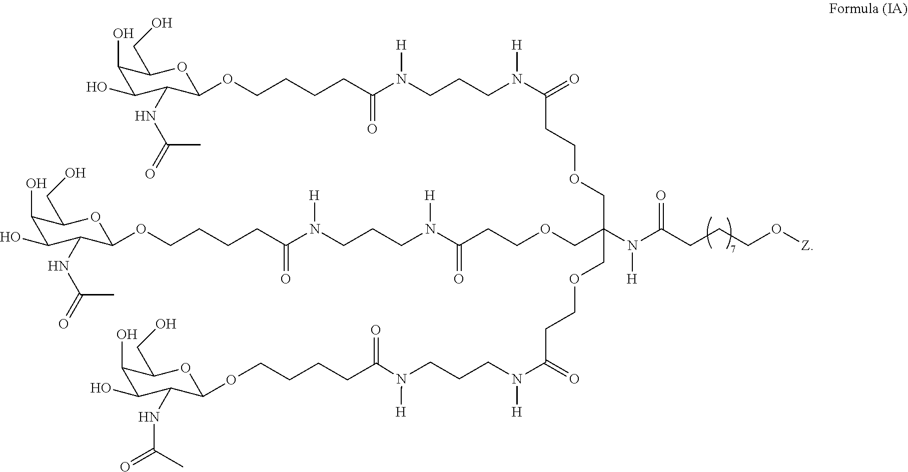

In some cases, the modified oligonucleotide has a structure of Formula (IIA):

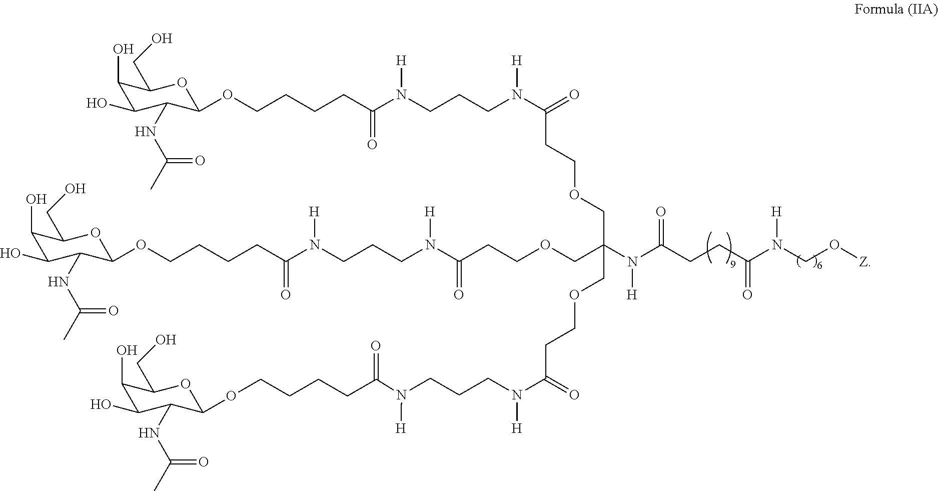

In some cases, the modified oligonucleotide has a structure of Formula (IIIA):

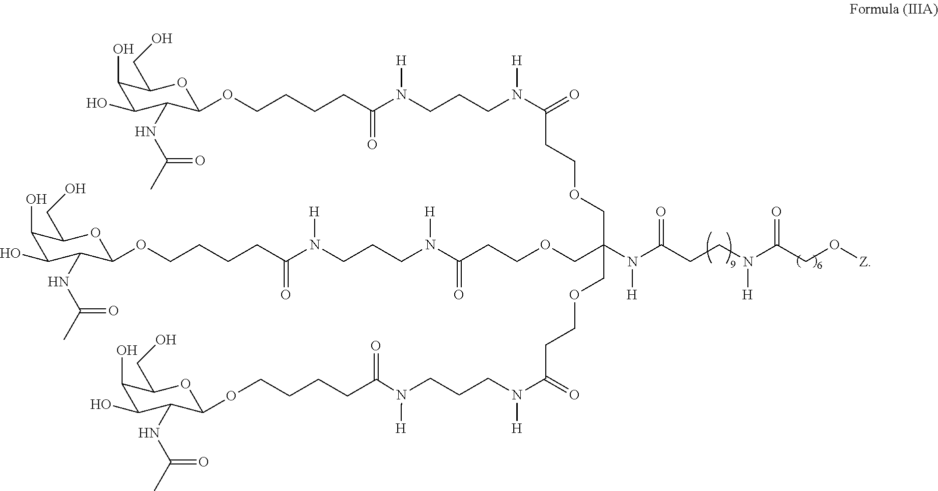

In some cases, the modified oligonucleotide has a structure of Formula (IVA):

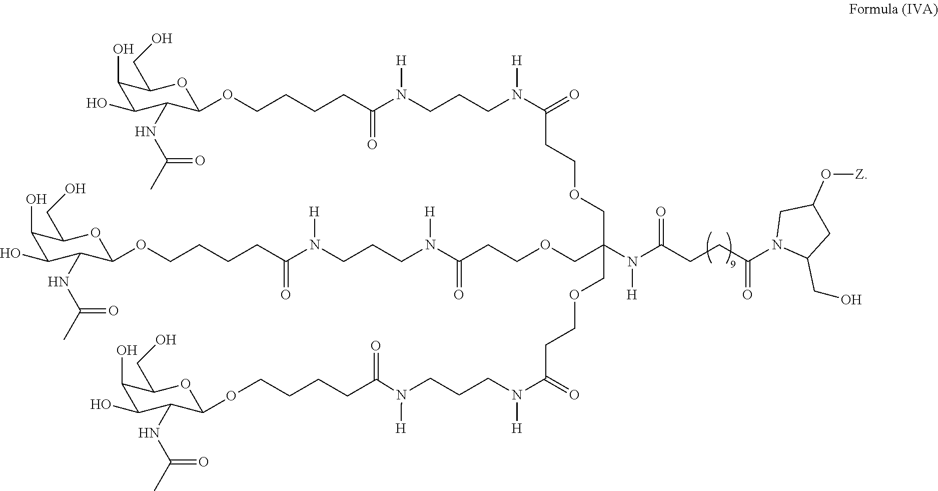

In some cases, the modified oligonucleotide has a structure of Formula VA:

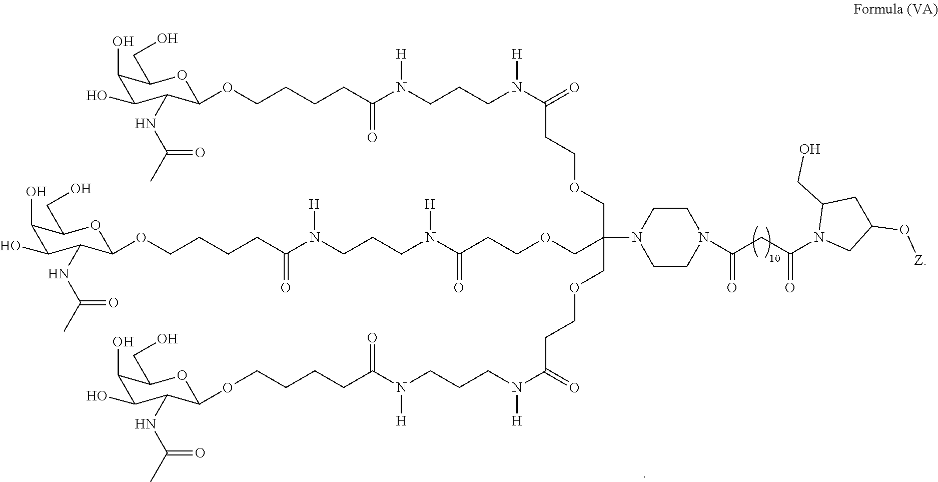

In some cases, the modified oligonucleotide has a structure of Formula (IA), (IIA), (IIIA), (IVA), or (VA). In some cases, the modified oligonucleotide has a structure of Formula (IA), (IIA), (IVA), or (VA). In some cases, the modified oligonucleotide has a structure of Formula (IA), (IIIA), (IVA), or (VA). In some cases, the modified oligonucleotide has a structure of Formula (IIA) or (IIIA).

### Oligonucleotides

For the compounds as disclosed herein (compounds for Formulae (I), (II), (III), (IV) and (V)), Z is an oligonucleotide comprising a sugar moiety, a nucleobase, and an internucleotide linkage. In some cases, Z comprises 10 to 300 nucleotides, each nucleotide comprising a sugar moiety, a nucleobase, and an internucleotide linkage. In some cases, Z comprises 25-100 nucleotides. In some cases, Z comprises 30-50 nucleotides.

The oligonucleotides disclosed herein in all instances include one or more GalNAc modifications as described herein. The GalNAc modification is particularly contemplated to be a triantennary GalNAc moiety having a structure described herein, i.e., as shown in Formula (I), (II), (III), (IV), or (V) (specifically, the portion of each of Formula (I), (II), (III), (IV), and (V) excluding the “Z” moiety). The GalNAc modification can be located at the 3′ end of the oligonucleotide, the 5′ end of the oligonucleotide, or both the 3′ and 5′ end of the oligonucleotide. In some cases, Z is an oligonucleotide modified at the 5′ end. In some cases, Z is an oligonucleotide modified at the 3′ end. In some cases, the modified oligonucleotide is a 3′ GD oligonucleotide (i.e., an oligonucleotide of Formula (I) where the decanol GalNAc moiety is located at the 3′ end of the oligonucleotide Z). In some cases, the modified oligonucleotide is a 5′ GD oligonucleotide (i.e., an oligonucleotide of Formula (I) where the decanol GalNAc moiety is located at the 5′ end of the oligonucleotide Z). In some cases, the modified oligonucleotide is a 3′ GE oligonucleotide (i.e., an oligonucleotide of Formula (II) where the extended GalNAc moiety is located at the 3′ end of the oligonucleotide Z). In some cases, the modified oligonucleotide is a 5′ GE oligonucleotide (i.e., an oligonucleotide of Formula (II) where the extended GalNAc moiety is located at the 5′ end of the oligonucleotide Z). In some cases, the modified oligonucleotide is a 3′ GE oligonucleotide of Formula (III) (i.e., an oligonucleotide of Formula (III) where the extended GalNAc moiety is located at the 3′ end of the oligonucleotide Z). In some cases, the modified oligonucleotide is a 5′ GE oligonucleotide of Formula (III) (i.e., an oligonucleotide of Formula (III) where the extended GalNAc moiety is located at the 5′ end of the oligonucleotide Z). In some cases, the modified oligonucleotide is a 3′ GP oligonucleotide (i.e., an oligonucleotide of Formula (IV) where the prolinol GalNAc moiety is located at the 3′ end of the oligonucleotide Z). In some cases, the modified oligonucleotide is a 5′ GP oligonucleotide (i.e., an oligonucleotide of Formula (IV) where the prolinol GalNAc moiety is located at the 5′ end of the oligonucleotide Z).

The present disclosure provides for oligonucleotides comprising unmodified or modified nucleotides or combinations thereof. As described herein “nucleoside” is defined as a compound containing a sugar moiety as described herein in combination with a nucleobase as described herein. A “nucleotide” is defined as a nucleoside including an internucleotide linkage as described herein. The modified nucleotides may by synthesized by any useful method (e.g., chemically, enzymatically, or recombinantly to include one or more modified or non-natural nucleosides).

The oligonucleotides described herein can include various modifications from naturally occurring oligonucleotides. As used herein, when referring to an oligonucleotide, the terms “chemical modification” or, as appropriate, “chemically modified” refer to modification with respect to a nucleobase (e.g., adenosine (A), guanosine (G), uridine (U), thymidine (T) or cytidine (C)), or to a sugar moiety (e.g., ribo- or deoxyribose), or to the internucleotide linkages in one or more of their positions. Generally, herein, these terms are not intended to refer to modifications found in naturally occurring oligonucleotides. In some embodiments, one or more of the nucleotides of an oligonucleotide described herein is chemically modified to enhance stability or other beneficial characteristics. Without being bound by theory, it is believed that certain modification can increase nuclease resistance and/or serum stability or decrease immunogenicity. For example, oligonucleotides described herein may contain nucleotides found to occur naturally in DNA or RNA (e.g., adenine, thymidine, guanosine, cytidine, uridine, or inosine) or may contain nucleotides that have one or more chemical modifications to one or more components of the nucleotide (e.g., the nucleobase, sugar, or internucleotide linkage) as described herein.

Modifications of the oligonucleotides of the disclosure include, but are not limited to, those listed in detail below. The oligonucleotides may comprise modifications which are naturally occurring, non-naturally occurring or the oligonucleotides can comprise both naturally and non-naturally occurring modifications.

The oligonucleotides of the disclosure can include any modification, such as to the sugar, the nucleobase, or the internucleotide linkage (e.g., to a linking phosphate, to a phosphodiester linkage, or to the phosphodiester backbone), described in detail below. For example, one or more atoms of a pyrimidine or purine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleotide linkage. Modifications according to the present disclosure may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

Unnatural and/or modified nucleotides may be introduced to oligonucleotides during synthesis or post-synthesis of the chains to achieve desired functions or properties. The modifications may be on internucleotide linkages, the nucleobases, or sugars. The modifications may be introduced at the terminal of a chain or anywhere else in the chain; with chemical synthesis or with a polymerase enzyme. Any of the regions of the oligonucleotides may be chemically modified.

### Sugar Moieties

Standard sugar moieties include ribose and deoxyribose. In some embodiments, the sugar moiety of one or more nucleotides of oligonucleotides disclosed herein comprise a ribose or modified ribose moiety. In some embodiments, the sugar moiety in the oligonucleotides may be a ribose optionally having a 2′-O-methyl, 2′-O-MOE, 2′-F, 2′-amino, 2′-O-propyl, 2′-aminopropyl, or 2′-OH modification. In some embodiments, the oligonucleotides disclosed herein comprise a deoxyribose or modified deoxyribose moiety. Alternative sugar moieties include other unnatural and natural sugars and sugar analogs as described herein, including sugar moieties comprising modifications or substitutions.

Alternative sugar moieties also include substituted sugar moieties. The oligonucleotides disclosed herein can comprise substituted furanose sugar moieties having one or more of the following at the 2′-position: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include —O[(CH₂)ₙO]ₘCH₃, —O(CH₂)ₙOCH₃, —O(CH₂)ₙ—NH₂, —O(CH₂)ₙCH₃, —O(CH₂)ₙ—ONH₂, and —O(CH₂)ₙ—ON[(CH₂)ₙCH₃]₂, where n and m are from 1 to about 10. In other embodiments, oligonucleotides disclosed herein can comprise substituted furanose sugar moieties having one or more of the following at the 2′ position: C₁ to C₁₀ alkyl, substituted C₁ to C₁₀ alkyl (e.g., substituted with one or more of OH, halo, amino, alkoxy, or thiol, or combinations thereof), alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes a 2′-methoxyethoxy (2′-O—CH₂CH₂OCH₃, also known as 2′-O-(2-methoxyethyl) or 2′-O-MOE) i.e., an alkoxy-alkoxy group. 2′-O-MOE nucleosides confer several beneficial properties to oligonucleotides including, but not limited to, increased nuclease resistance, improved pharmacokinetics properties, reduced non-specific protein binding, reduced toxicity, reduced immunostimulatory properties, and enhanced target affinity as compared to unmodified oligonucleotides.

Another exemplary alternative sugar moiety comprises a 2′-dimethylaminooxyethoxy group, i.e., a —O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—(CH₂)₂—O—(CH₂)₂—N(CH₃)₂. Further exemplary alternative sugar moieties include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other alternatives include 2′-methoxy (2′-OCH₃), 2′-aminopropoxy (2′-OCH₂CH₂CH₂NH₂) and 2′-fluoro (2′-F). Similar modifications can also be made at other positions on the sugar moieties of an oligonucleotide, particularly the 3′ position of the sugar on the 3′ terminal nucleotide or in 2′-5′ linked oligonucleotides and the 5′ position of 5′ terminal nucleotide. Oligonucleotides can also have sugar analogs or mimetics such as cyclobutyl moieties in place of a pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An oligonucleotide of the disclosure can include one or more bicyclic sugar moieties. A “bicyclic sugar” is a furanosyl ring modified by the bridging of two atoms. A “bicyclic nucleoside” (“BNA”) is a nucleoside having a sugar moiety including a bridge connecting two carbon atoms of the sugar ring, thereby forming a bicyclic ring system. In certain embodiments, the bridge connects the 4′-carbon and the 2′-carbon of the sugar ring. Thus, in some embodiments an oligonucleotide of the disclosure may include one or more locked nucleosides. A locked nucleoside is a nucleoside having a modified ribose moiety in which the ribose moiety includes an extra bridge connecting the 2′ and 4′ carbons. In other words, a locked nucleoside is a nucleoside including a bicyclic sugar moiety including a 4′-CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193). Examples of bicyclic nucleosides for use in the oligonucleotides of the disclosure include without limitation nucleosides including a bridge between the 4′ and the 2′ ribosyl ring atoms. In certain embodiments, the oligonucleotides of the disclosure include one or more bicyclic nucleosides including a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference. In some embodiments, at least 1 of the alternative sugar moieties is a BNA (e.g., an LNA), such as at least 2, such as at least 3, at least 4, at least 5, at least 6, at least 7, or at least 8 of the alternative sugar moieties are BNAs. In a still further embodiment, all the alternative moieties are BNAs.

Additional representative U.S. patents and US patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

An oligonucleotide of the disclosure may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an oligonucleotide of the disclosure includes one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The sugar moiety may also be modified with a cyclopropane ring to produce a tricyclodeoxynucleic acid (tricyclo DNA). The sugar moiety may be e.g., 1,5,-anhydrohexitol, threose to produce a threose nucleoside (TNA), or arabinose to produce an arabino nucleoside.

The sugar moiety can also be a non-sugar such as cyclohexene to produce cyclohexene nucleic acid (CeNA) or glycol to produce glycol nucleic acids (GNA). Potentially stabilizing modifications to the ends of nucleotide molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Exemplary oligonucleotides of the disclosure include standard and/or alternative sugar moieties in any combination and at various positions, and may also include DNA or RNA oligonucleotides. Incorporation of modified sugar moieties into the oligonucleotide of the disclosure may enhance the affinity of the oligonucleotide for the target nucleic acid.

In some embodiments, the sugar moieties may be independently, for each position, selected from ribose and deoxyribose, and/or may comprise modifications such as but not limited to 2′-O-alkyl, 2′-O-methoxyethyl, 2′-O-allyl, 2′-O-alkalamine, 2′-fluororibse 2′-deoxyribase, and locked nucleic acid (LNA). In some embodiments, the oligonucleotides include one or more modified sugar moieties, e.g., 2′-modified modified sugar moieties. In some embodiments, the oligonucleotides described herein include one or more 2′-modified sugar moieties independently selected from the group consisting of 2′-O-alkyl-RNA (e.g., 2′-O-methyl-RNA), 2′-alkoxy-RNA, 2′-O-methoxyethyl-RNA, 2′-amino-DNA, 2′-fluoro-DNA, arabinose nucleic acid (ANA), 2′-fluoro-ANA, and bridged nucleic acid (“BNA” e.g., locked nucleic acid “LNA”) moieties. In some embodiments, the one or more modified sugar moiety is a BNA. In some cases, at least one sugar moiety is a ribose or modified ribose sugar. In some cases, at least one sugar moiety is a ribose sugar. In some cases, at least one sugar moiety is a modified ribose sugar. In some cases, at least one sugar moiety is independently 2′-methoxy-ribose, 2′-MOE-ribose, 5′-methyl-2′deoxyribose, 2′-deoxy-2′-fluororibose, 2′-fluoro-arabinose, 2-methoxy-arabinose, 2′deoxyribose, a locked nucleic acid (LNA), or a deoxyhexose. In some cases, each sugar moiety is independently 2′-methoxy-ribose, 2′-MOE-ribose, 5′-methyl-2′deoxyribose, 2′-deoxy-2′-fluororibose, 2′-fluoro-arabinose, 2-methoxy-arabinose, 2′deoxyribose, a locked nucleic acid (LNA), or a deoxyhexose.

In some embodiments, the oligonucleotides described herein include phosphorodiamidate morpholino oligomers (PMO), in which a deoxyribose moiety is replaced by a morpholine ring, and the charged phosphodiester inter-subunit linkage is replaced by an uncharged phosphorodiamidate linkage, as described in Summerton, et al., Antisense Nucleic Acid Drug Dev. 1997, 7:63-70. In some instances, the oligonucleotides include modified sugar moieties comprising bicyclic sugar derivatives (LNA, ENA, CLNA, CENA, AENA etc.), acyclic sugar analogs (UNA, PNA, etc.) or analogs containing sugars other than ribose or deoxyribose (e.g., a pyranose ring (ANA, hexitol nucleic acid (HNA)).

In some embodiments, the oligonucleotide includes at least 1 modified sugar moiety, such as at least 2, at least 3, at least 4, at least 5, at least 6, at least 7, at least 8, at least 9, at least 10, at least 11, at least 12, at least 13, at least 14, at least 15 or at least 16 modified sugar moieties. In other embodiments, the oligonucleotides include from 1 to 10 modified sugar moieties, such as from 2 to 9 modified sugar moieties, such as from 3 to 8 modified sugar moieties, such as from 4 to 7 modified sugar moieties, such as 6 or 7 modified sugar moieties. In some embodiments, the oligonucleotide contains 1% to 100% modified sugar moieties (either in relation to overall sugar moiety content, or in relation to one or more types of sugar moiety, e.g., any one or more of ribose, deoxyribose, or derivatives thereof) or any intervening percentage (e.g., 1% to 5%, 1% to 10%, 1% to 20%, 1% to 25%, 1% to 50%, 1% to 60%, 1% to 70%, 1% to 80%, 1% to 90%, 1% to 95%, 10% to 20%, 10% to 25%, 10% to 50%, 10% to 60%, 10% to 70%, 10% to 80%, 10% to 90%, 10% to 95%, 10% to 100%, 20% to 25%, 20% to 50%, 20% to 60%, 20% to 70%, 20% to 80%, 20% to 90%, 20% to 95%, 20% to 100%, 50% to 60%, 50% to 70%, 50% to 80%, 50% to 90%, 50% to 95%, 50% to 100%, 70% to 80%, 70% to 90%, 70% to 95%, 70% to 100%, 80% to 90%, 80% to 95%, 80% to 100%, 90% to 95%, 90% to 100%, and 95% to 100%). It will be understood that any remaining sugar moieties in the oligonucleotides are ribose or deoxyribose sugar moieties.

In some embodiments, the sugar moiety has the structure of any one of Formula AA-AE:

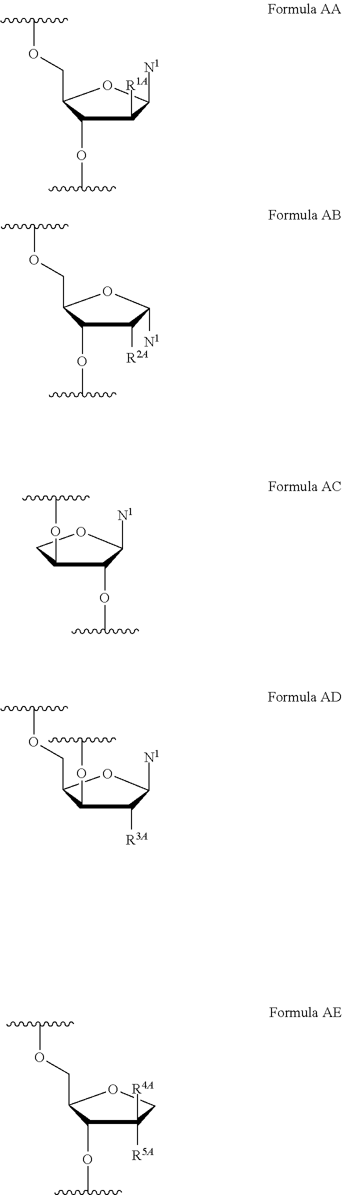

wherein N¹ is hydrogen or a nucleobase;
R^{1A} is hydroxy, halogen, or C₁-C₆ alkoxy;
R^{2A} is hydrogen, hydroxy, halogen, or C₁-C₆ alkoxy;
R^{3A} is hydrogen, hydroxy, halogen, or C₁-C₆ alkoxy;
R^{4A} is hydrogen, hydroxy, halogen, or C₁-C₆ alkoxy; and
R^{5A} is hydrogen, hydroxy, halogen, or C₁-C₆ alkoxy.

In some embodiments, the sugar moiety has the structure of any one of Formula BA-BF:

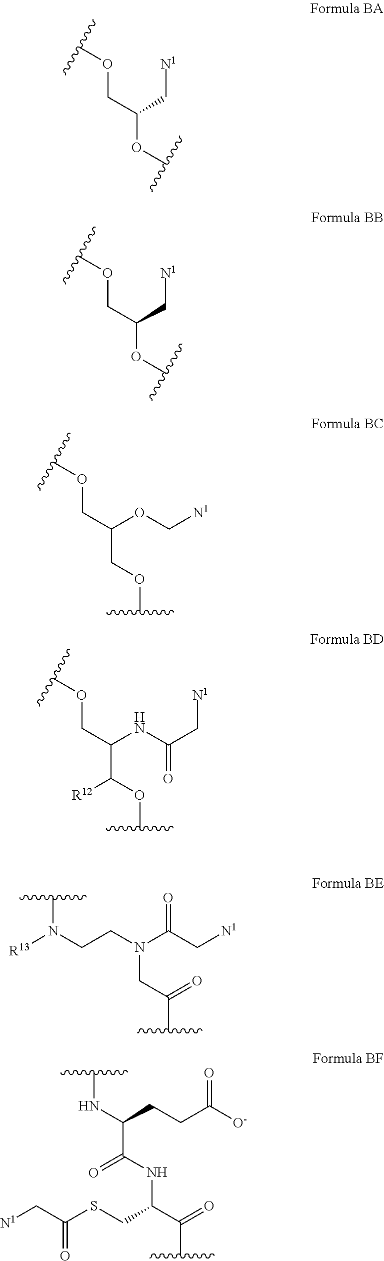

wherein N¹ is hydrogen or a nucleobase;
R¹² is hydrogen, hydroxy, fluoro, halogen, C₁-C₆ alkyl, C₁-C₆ heteroalkyl, or C₁-C₆ alkoxy; and
R¹³ is hydrogen or C₁-C₆ alkyl.

In some embodiments, the sugar moiety has the structure of any one of Formula CA-CD:

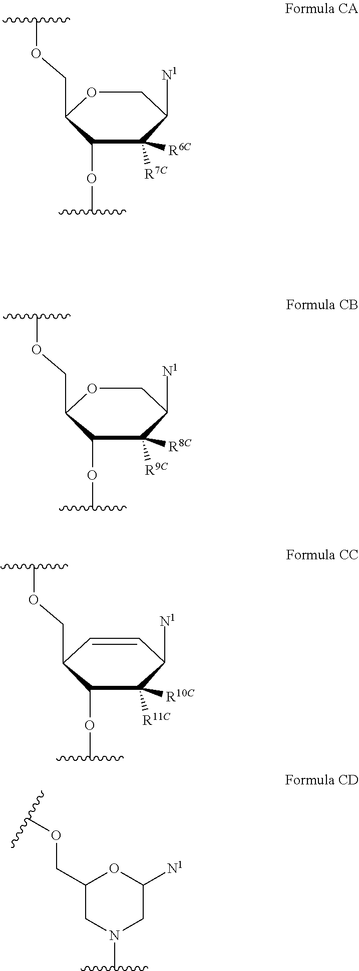

wherein N¹ is hydrogen or a nucleobase;
R^{6C} is hydrogen, hydroxy, or halogen;
R^{7C} is hydrogen, hydroxy, halogen, or C₁-C₆ alkoxy;
R^{8C} is hydrogen or halogen;
R^{9C} is hydrogen or hydroxy, halogen, or C₁-C₆ alkoxy;
R^{10C} Is hydrogen or halogen; and
R^{11C} is hydrogen or hydroxy, halogen, or C₁-C₆ alkoxy.

Non-limiting further examples of suitable sugars for use in the oligonucleotides disclosed herein include those disclosed in WO 2020/154342, WO 2020/154344, and WO 2020/154343, each of which is incorporated herein by reference in its entirety.

In some cases, at least one sugar moiety is β-D-2′,3′-dideoxyglucopyranose (β-homo-RNA), 2′-methoxyribose, 2′-methoxyethylribose (2′-MOE-ribose), 2′-fluororibose, 5′-methyl-2′deoxyribose, 2′-deoxy-2′-fluororibose, 2′-fluoroarabinose, 2-methoxy-arabinose, 2′deoxyribose, a locked nucleic acid (LNA), constrained ethyl (cEt), bridged nucleic acid (BNA), or deoxyhexose. In some cases, at least 10% of the sugar moieties are β-D-2′,3′-dideoxyglucopyranose (β-homo-RNA), 2′-methoxyribose, 2′-methoxyethylribose (2′-MOE-ribose), 5′-methyl-2′deoxyribose, 2′-deoxy-2′-fluororibose, 2′-fluoroarabinose (FANA), 2-methoxy-arabinose, 2′deoxyribose, a locked nucleic acid (LNA), constrained ethyl (cEt), bridged nucleic acid (BNA), or deoxyhexose.

### Internucleotide Linkages

The oligonucleotides disclosed herein include one or more internucleotide linkages. The remaining internucleotide linkage(s) of the disclosed oligonucleotides can be unmodified or natural internucleotide linkages and/or alternative nucleotide linkages. Unmodified or natural internucleotide linkages can be a phosphate linkage. Alternative internucleotide linkages include unnatural internucleotide linkages and/or modified natural internucleotide linkages. Examples of alternative internucleotide linkages are known in the art, including, but not limited to, phosphorothioate, boronophosphate, phosphotriester, phosphorothionate, or phosphoramidate linkages, and other variants of the phosphate backbone. In some embodiments, the oligonucleotides of the disclosure include at least one alternative internucleotide linkage.

In some instances, the phosphate group (PO) of a natural internucleotide linkage can be replaced with a phosphorothioate (PS) or boranophosphonate (PB) group, or the 3′,5′-phosphodiester bond of a natural internucleotide linkage can be replaced with a 2′,5′-bond, or the ester bond can be replaced with an amide bond, etc. Some embodiments include oligonucleotides with heteroatom backbones, and in particular —CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—PO—CH₂—] of the above-referenced U.S. Pat. No. 5,489,677, and the amide backbones of the above-referenced U.S. Pat. No. 5,602,240. In some embodiments, the oligonucleotides featured herein have morpholino backbone structures of the above-referenced U.S. Pat. No. 5,034,506. In some embodiments, the internucleotide linkages within a contiguous nucleotide sequence are alternative internucleotide linkages. In some embodiments, the internucleotide linkages within a contiguous nucleotide sequence are phosphoroamidate linkages. In some embodiments the alternative internucleotide linkages are stereochemically pure alternative phosphoroamidate linkages. In some embodiments, the alternative internucleotide linkages are Sp phosphoroamidate linkages. In other embodiments, the alternative internucleotide linkages are Rp phosphoroamidate linkages.

Other alternatives chemistries for the internucleotide linkages of the oligonucleotides described herein include a 5′ phosphate or 5′ phosphate mimic, e.g., a 5-terminal phosphate or phosphate mimic of an oligonucleotide. Suitable phosphate mimics are disclosed in, for example US Patent Publication No. 2012/0157511, the entire contents of which are incorporated herein by reference.

In some cases, at least one internucleotide linkage comprises a phosphorothioate or phosphoramidate linkage. In some cases, at least 10% of the internucleotide linkages of the passenger oligonucleotide comprise phosphorothioate or phosphoramidate linkages. In some cases, the phosphoroamidate is a mesyl phosphoramidate with mesyl azide (PA-1), an isopropylsulfonylphosporamidate, or a cyclopropylsulfonylphosphoramidate.

In some cases, the oligonucleotide comprises one PAX internucleotide linkage. In some cases, the oligonucleotide comprises 2-10 PAX internucleotide linkages. The nucleotides herein can also include other modified internucleotide linkages. In some cases, at least one internucleotide linkage is a phosphorothioate (PS). In some cases, at least one internucleotide linkage is a mesyl phosphoramidate (PA1).

In some instances, the PAX internucleotide linkage has a structure shown in Table 1:

TABLE 1

| | Structure |
|---|---|
| L1 | 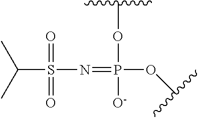 [SMILES: *OP([O-])(=NS(=O)(=O)C(C)C)O*] |
| L2 | 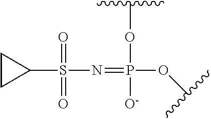 [SMILES: *OP([O-])(=NS(=O)(=O)C1CC1)O*] |
| L3 | 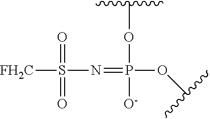 |
| L4 | 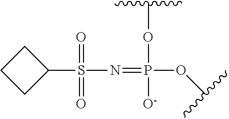 [SMILES: *OP([O-])(=NS(=O)(=O)C1CCC1)O*] |
| L5 | 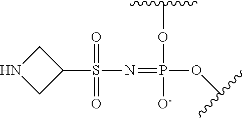 [SMILES: *OP([O-])(=NS(=O)(=O)C1CNC1)O*] |
| L6 | 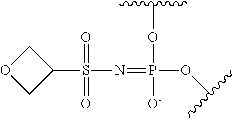 [SMILES: *OP([O-])(=NS(=O)(=O)C1COC1)O*] |
| L7 | 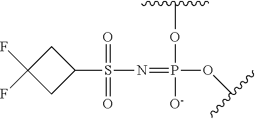 [SMILES: *OP([O-])(=NS(=O)(=O)C1CC(F)(F)C1)O*] |
| L8 | 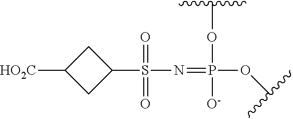 [SMILES: *OP([O-])(=NS(=O)(=O)C1CC(C(=O)O)C1)O*] |
| L9 | 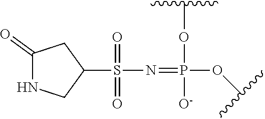 [SMILES: *OP([O-])(=NS(=O)(=O)C1CNC(=O)C1)O*] |
| L10 | 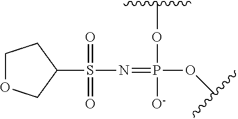 [SMILES: *OP([O-])(=NS(=O)(=O)C1CCOC1)O*] |
| L11 | 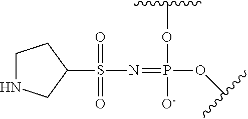 [SMILES: *OP([O-])(=NS(=O)(=O)C1CCNC1)O*] |
| L12 | 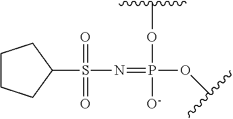 [SMILES: *OP([O-])(=NS(=O)(=O)C1CCCC1)O*] |
| L13 | 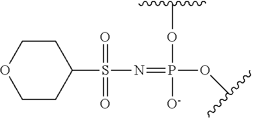 [SMILES: *OP([O-])(=NS(=O)(=O)C1CCOCC1)O*] |
| L14 | 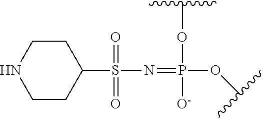 [SMILES: *OP([O-])(=NS(=O)(=O)C1CCNCC1)O*] |
| L15 | 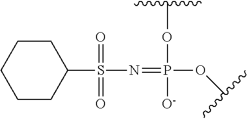 [SMILES: *OP([O-])(=NS(=O)(=O)C1CCCCC1)O*] |
| L16 | 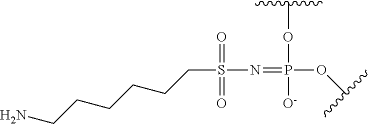 [SMILES: *OP([O-])(=NS(=O)(=O)CCCCCCN)O*] |
| L17 | 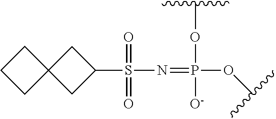 [SMILES: *OP([O-])(=NS(=O)(=O)C1CC2(CCC2)C1)O*] |

### Nucleobases

Oligonucleotides described herein can also include one or more nucleobases (often referred to in the art simply as “bases”), including one or more standard nucleobases and/or alternative nucleobases (e.g., unnatural nucleobases, or natural nucleobases comprising modifications or substitutions). In some cases, at least one nucleotide in the oligonucleotides disclosed herein does not have a nucleobase—i.e., is an abasic oligonucleotide.

Standard nucleobases include the purine bases adenine (A) and guanine (G), and the pyrimidine bases thymine (T), cytosine (C), and uracil (U). Alternative nucleobases include other unnatural/synthetic and natural nucleobases as described herein, including nucleobases comprising modifications or substitutions.

In some instances, the oligonucleotides comprise at least one unnatural nucleobase. In some instances, the oligonucleotides comprise at least one modified natural nucleobase. The terms “modified” or, as appropriate, “modification” refer to structural and/or chemical modifications with respect to A, G, U, T, or C nucleobases, nucleosides, and/or nucleotides. Nucleotides in the oligonucleotides of the present disclosure may comprise non-standard nucleotides, such as non-naturally occurring nucleotides (“unnatural nucleotides”) or chemically synthesized nucleotides. One or more atoms of a nucleobase may be replaced or substituted with optionally substituted amino, optionally substituted thiol, optionally substituted alkyl (e.g., methyl or ethyl), or halo (e.g., chloro or fluoro).

Nonlimiting examples of synthetic and natural nucleobases that can serve as alternative nucleobases include 5-methylcytosine, 5-hydroxymethylcytosine, 5-formylcytosine, 5-carboxycytosine, pyrrolocytosine, dideoxycytosine, uracil, 5-methoxyuracil, 5-hydroxydeoxyuracil, dihydrouracil, 4-thiouracil, pseudouracil, 1-methylpseudouracil, deoxyuracil, 5-hydroxybutynl-2′-deoxyuracil, xanthine, hypoxanthine, 7-deaza-xanthine, thienoguanine, 8-aza-7-deazaguanine, 7-methylguanine, 7-deazaguanine, 6-aminomethyl-7-deazaguanine, 8-aminoguanine, 2,2,7-trimethylguanine, 8-methyladenine, 8-azidoadenine, 7-methyladenine, 7-deazaadenine, 3-deazaadenine, 2,6-diaminopurine, 2-aminopurine, 7-deaza-8-aza-adenine, 8-amino-adenine, thymine, dideoxythymine, 5-nitroindole, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 8-azaguanine and 8-azaadenine, and 3-deazaguanine. In a some embodiments the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methylcytosine, 5-thiozolo-cytosine, 5-propynyl-cytosine, 5-propynyl-uracil, 5-bromouracil, 5-thiazolo-uracil, 2-thio-uracil, pseudouracil, 1-methylpseudouracil, 5-methoxyuracil, 2′-thio-thymine, hypoxanthine, diaminopurine, 6-aminopurine, 2-aminopurine, 2,6-diaminopurine, and 2-chloro-6-aminopurine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, those disclosed by Englisch et al., (1991) Angewandte Chemie, International Edition, 30:613, those disclosed by Sanghvi, Y S., Chapter 15, Antisense Research and Applications, pages 289-302, Crooke, S. T. and Lebleu, B., Ed., CRC Press, 1993, in Hirao et al (2012) Accounts of Chemical Research vol 45, page 2055 and Bergstrom (2009) Current Protocols in Nucleic Acid Chemistry Suppl. 37 1.4.1. Certain of these nucleobases are particularly useful for increasing the binding affinity of the oligonucleotides described herein. These include 5-substituted pyrimidines, 6-azapyrimidines, and N-2, N-6 and 0-6 substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are exemplary base substitutions.

In other embodiments, the nucleobases in the oligonucleotides may be independently, for each position, selected from adenine, uridine, guanine, or cytidine or analogs of adenine, uridine, guanine, or cytidine, such as modified adenine, uridine, guanine, or cytidine. Non-limiting examples of adenine, uridine, guanine, and cytidine analogs and modified adenine, uridine, guanine, and cytidine include N6-methyladenine, N1-methylademine, N6-2′-0-dimehtyladenosine, pseudouridine, N1-methypseudouridine, 5-iodouridine, 4-thiouridine, 2-thiouridine, 5-methyluridine, pseudoisocytosine, 5-methoxycytosine, 2-thiocytosine, 5-hydroxycytosine, N4-methylcytosine, 5-hydroxymethylcytosine, hypoxanthine, N1-methylguanine, 06-methylguanine, 1-methyl-guanosine, N2-methyl-guanosine, N2,N2-dimethyl-guanonsine, 2-methyl-guanosine, N7-methyl-guanosine, 1-methyl-guanosine, N2,N7-dimethyl-guanosine, and isoguanine. For example, uridine (U) may be replaced with pseudouridine (ip), 2-thiouridine (s2U), dihydrouridine (D), 5-bromo-U, 5-iodo-U, etc. A purine may be replaced with a 2,6-diaminopurine.

Representative U.S. patents that teach the preparation of certain of the above noted nucleobases as well as other nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

Additional nucleobases found in the oligonucleotides disclosed herein include, but are not limited to, the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 21-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-luoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 21-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; a-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,21-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-I-deaza-pseudoisocytidine; 1-methyl-pseudoisocytidine; 2-methoxy-5-methyl-cytidine; 2-methoxy-cytidine; 2-thio-5-methyl-cytidine; 4-methoxy-I-methyl-pseudoisocytidine; 4-methoxy-pseudoisocytidine; 4-thio-I-methyl-1-deaza-pseudoisocytidine; 4-thio-I-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 21-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; a-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-ethyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-ethyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydrouridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; N1-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-PentenylaminomethyOuridine TP; 5-propynyl uracil; a-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 1-Ethyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-I-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-I-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; P seudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-I-methyl-1-deaza-pseudouridine; 2-thio-I-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-I-methyl-pseudouridine; 4-thio-pseudouridine; 5-aza-uridine; Dihydropseudouridine; (±)1-(2-Hydroxy propyl)pseudouridine TP; (2R)—1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bi s-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxy butyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxy benzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxy benzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy-ethoxy)-propionyl]pseudouridine TP; 1-13-[2-(2-Amino ethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-rifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-12-(2-[2-(2-ethoxy)-ethoxy]-ethoxy)-ethoxy1]propionic acid; Pseudouridine TP 1-[3-{24242-12(2-ethoxy)-ethoxy 1-ethoxy]-ethoxy)-ethoxy1]propionic acid; Pseudouridine TP 1-[3-12-(2-[2-ethoxy]-ethoxy)-ethoxyllpropionic acid; Pseudouridine TP 143-12-(2-ethoxy)-ethoxyll propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-I-yl: 1,3-(diaza)-2-(oxo)-phenthiazin-I-y1; 1,3-(diaza)-2-(oxo)-phenoxazin-I-y1; 1,3,5-(triaza)-2,6-(dioxa)-naphthalene; 2 (amino)purine; 2,4,5-(trimethyl)phenyl; 2′ methyl, 2′amino, 2′azido, 2′fluro-cytidine; 21 methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-y1; 7-(aminoalkylhydroxy)-1-(aza)-2-(thio)-3-(aza)-phenthiazin-I-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-I-y1; 7-(guanidiniumalkylhydroxy)-1-(aza)-2-(thio)-3-(aza)-phenoxazin-I-y1; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-y1; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; O6-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-y1; para-substituted-6-phenyl-pyrrolo-pyrimidin-2-on-3-y1; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP. In some embodiments, the alternative nucleobases are selected from the group consisting of pseudouridine (ψ), 2-thiouridine (s2U), 4′-thiouridine, 5-methylcytosine, 2-thio-I-methyl-1-deaza-pseudouridine, 2-thio-I-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-I-methyl-pseudouridine, 4-thio-pseudouridine, 5-aza-uridine, dihydropseudouridine, 5-methyluridine, 5-methoxyuridine, 21-O-methyl uridine, 1-methyl-pseudouridine (m1ψ), 1-ethyl-pseudouridine (e1ψ), 5-methoxy-uridine (mo5U), 5-methyl-cytidine (m5C), a-thio-guanosine, a-thio-adenosine, 5-cyano uridine, 4′-thio uridine 7-deaza-adenine, 1-methyl-adenosine (ml A), 2-methyl-adenine (m2A), N6-methyl-adenosine (m6A), and 2,6-Diaminopurine, (I), 1-methyl-inosine (m1l), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (ml G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropy1)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethy1-2-thiouridine, 5-carboxymethylaminomethy1-2-geranylthiouridine, 5-carboxymethylaminomethy1-2-selenouridine, 5-cyanomethyluridine, 5-hydroxycytidine, 5-methylaminomethy1-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethy1-2-thiouridine, geranylated 5-carboxymethylaminomethy1-2-thiouridine, Qbase, preQObase, preQ1base, and two or more combinations thereof. In some embodiments, the alternative nucleobase is selected from the group consisting of pseudouridine, 1-methyl-pseudouridine, 1-ethyl-pseudouridine, 5-methylcytosine, 5-methoxyuridine, and a combination thereof.

In some embodiments, the nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethy1-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (Tm5U), 1-taurinomethyl-pseudouridine, 5-taurinomethy1-2-thio-uridine(Tm5s2U), 1-taurinomethyl-4-thio-pseudouridine, 5-methyl-uridine (m5U, i.e., having the nucleobase deoxythymine), 1-methyl-pseudouridine (m1 ψ), 1-ethyl-pseudouridine (e1ψ), 5-methyl-2-thio-uridine (m5s2U), 1-methyl-4-thio-pseudouridine (m1s4 ψ), 4-thio-I-methyl-pseudouridine, 3-methyl-pseudouridine (m3 ψ), 2-thio-1-methyl-pseudouridine, 1-methyl-1-deaza-pseudouridine, 2-thio-I-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methy1-3-(3-amino-3-carboxypropyl)pseudouridine (acp3 kv), 5-(isopentenylaminomethyl)uridine (inm5U), 5-(isopentenylaminomethyl)-2-thio-uridine (inm5s2U), a-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethy1-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-I-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-I-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine(ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the nucleobase is a modified adenine. Exemplary nucleobases and nucleosides having a modified adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (miAm), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the nucleobase is a modified guanine. Exemplary nucleobases and nucleosides having a modified guanine include inosine (I), 1-methyl-inosine (mil), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQi), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine N2-methyl-guanosine (m2G), N2,N2-dimethyl-guanosine (m22G), N2,7-dimethyl-guanosine (m2′7G), N2, N2,7-dimethyl-guanosine 8-oxo-guanosine, 7-methyl-8-oxo-guanosine, 1-methyl-6-thio-guanosine, N2-methyl-6-thio-guanosine, N2,N2-dimethyl-6-thio-guanosine, a-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (miGm), N2,7-dimethyl-2′-O-methyl-guanosine (m2′7Gm), 2′-O-methyl-inosine (Im), 1,2′-O-dimethyl-inosine 2′-O-ribosylguanosine (phosphate) (Gr(p)), 1-thio-guanosine, 06-methyl-guanosine, 2′-F-ara-guanosine, and 2′-F-guanosine.

Non-limiting further examples of suitable alternative nucleobases, nucleosides, and nucleotides for use in the oligonucleotides disclosed herein include those disclosed in WO 2020/154342, WO 2020/154344, and WO 2020/154343, each of which is incorporated herein by reference in its entirety.

In some embodiments, the oligonucleotides are uniformly modified (e.g., fully modified, modified throughout the entire sequence) for a particular modification. For example, an oligonucleotide can be uniformly modified with 1-methyl-pseudouridine, meaning that all uridine residues in the oligonucleotide sequence are replaced with 1-methyl-pseudouridine. Similarly, an oligonucleotide can be uniformly modified for any type of nucleobase present in the sequence by replacement with an alternative nucleobase such as those set forth above.

The oligonucleotides of the present disclosure may be partially or fully modified along the entire length of the molecule. For example, one or more or all or a given type of nucleobase (e.g., purine or pyrimidine, or any one or more or all of A, G, U, T, or C) may be uniformly modified in an oligonucleotide of the disclosure, or in a given predetermined sequence region thereof (e.g., in an mRNA including or excluding the polyA tail). In some embodiments, all nucleotides X in an oligonucleotide of the present disclosure (or in a given sequence region thereof) are modified nucleotides, wherein X may any one of nucleotides A, G, U, T, C, or any one of the combinations A+G, A+U, A+C, G−HU, G−FC, U+C, A+G−HU, A+G−FC, G−HU+C or A+G+C.

In some embodiments, the oligonucleotide contains 1% to 100% modified nucleobases (either in relation to overall nucleobase content, or in relation to one or more types of nucleobase, i.e., any one or more of A, G, U, T, and/or C) or any intervening percentage (e.g., 1% to 5%, 1% to 10%, 1% to 20%, 1% to 25%, 1% to 50%, 1% to 60%, 1% to 70%, 1% to 80%, 1% to 90%, 1% to 95%, 10% to 20%, 10% to 25%, 10% to 50%, 10% to 60%, 10% to 70%, 10% to 80%, 10% to 90%, 10% to 95%, 10% to 100%, 20% to 25%, 20% to 50%, 20% to 60%, 20% to 70%, 20% to 80%, 20% to 90%, 20% to 95%, 20% to 100%, 50% to 60%, 50% to 70%, 50% to 80%, 50% to 90%, 50% to 95%, 50% to 100%, 70% to 80%, 70% to 90%, 70% to 95%, 70% to 100%, 80% to 90%, 80% to 95%, 80% to 100%, 90% to 95%, 90% to 100%, and 95% to 100%). It will be understood that any remaining percentage is accounted for by the presence of standard nucleobases A, G, T, U, and/or C.

The oligonucleotides may contain at a minimum 0% and at maximum 100% modified nucleobases, or any intervening percentage, such as at least 1% modified nucleobases, at least 5% modified nucleobases, at least 10% modified nucleobases, at least 25% modified nucleobases, at least 50% modified nucleobases, at least 80% modified nucleobases, or at least 90% modified nucleobases. For example, the oligonucleotides may contain a modified pyrimidine such as a modified uracil or cytosine. In some embodiments, at least 5%, at least 10%, at least 25%, at least 50%, at least 80%, at least 90% or 100% of the uracil in the oligonucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the oligonucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

In some embodiments, the oligonucleotides described herein includes at least 1 modified nucleobase, such as at least 2, at least 3, at least 4, at least 5, at least 6, at least 7, at least 8, at least 9, at least 10, at least 11, at least 12, at least 13, at least 14, at least 15 or at least 16 modified nucleobases. In other embodiments, the oligonucleotides include from 1 to 10 modified nucleobases, such as from 2 to 9 modified nucleobases, such as from 3 to 8 modified nucleobases, such as from 4 to 7 modified nucleobases, such as 6 or 7 modified nucleobases. In some embodiments, the oligonucleotides include a combination of at least two (e.g., 2, 3, 4 or more) of the aforementioned modified nucleobases.

### Oligonucleotides Conjugated to Additional Moieties

In some embodiments, an oligonucleotide as disclosed herein comprises one or more additional chemical moieties (or ligands). Various additional chemical moieties, e.g., targeting moieties, carbohydrate moieties, lipid moieties, etc. are known in the art and can be utilized in accordance with the present disclosure to modulate properties and/or activities of provided oligonucleotides, e.g., stability, half-life, activities, delivery, pharmacodynamics properties, pharmacokinetic properties. In some embodiments, certain additional chemical moieties facilitate delivery of oligonucleotides to desired cells, tissues and/or organs, including but not limited the cells of the central nervous system. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides. In some embodiments, an additional chemical moiety is or comprises a small molecule moiety. In some embodiments, a small molecule is a ligand of a protein (e.g., receptor). In some embodiments, a small molecule binds to a polypeptide. In some embodiments, a small molecule is an inhibitor of a polypeptide. In some embodiments, an additional chemical moiety is or comprises a peptide moiety (e.g., an antibody). In some embodiments, an additional chemical moiety is or comprises a nucleic acid moiety, such as a nucleic acid moiety which forms a duplex or other secondary structure with the original oligonucleotide chain (before conjugation) or a portion thereof. In some embodiments, a nucleic acid is or comprises an oligonucleotide targeting the same or a different target, and may perform its activity through the same or a different mechanism. In some embodiments, a nucleic acid is or comprises a RNAi agent. In some embodiments, a nucleic acid is or comprises a miRNA agent. In some embodiments, a nucleic acid is or comprises RNase H dependent. In some embodiments, a nucleic acid is or comprises a gRNA. In some embodiments, a nucleic acid is or comprises an aptamer. In some embodiments, an additional chemical moiety is or comprises a carbohydrate moiety as described herein. Many useful agents, e.g., small molecules, peptides, carbohydrates, nucleic acid agents, etc., may be conjugated with oligonucleotides herein in accordance with the present disclosure. In some embodiments, a provided oligonucleotide can comprise two or more additional chemical moieties, wherein the additional chemical moieties can be the same or different.

Oligonucleotides described herein may be chemically linked to one or more ligands, moieties, or conjugates that enhance the activity, cellular distribution, or cellular uptake of the oligonucleotide. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In some embodiments, a ligand alters the distribution, targeting, or lifetime of an oligonucleotide agent into which it is incorporated. In some embodiments, a ligand provides an enhanced affinity for a selected target, e.g., molecule, cell or cell type, compartment, e.g., a cellular or organ compartment, tissue, organ, or region of the body, as, e.g., compared to a species absent such a ligand.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salts of a polyamine, or alpha helical peptides.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

In some embodiments, an additional chemical moiety is a targeting moiety. In some embodiments, an additional chemical moiety is or comprises a carbohydrate moiety. In some embodiments, an additional chemical moiety is or comprises a lipid moiety. In some embodiments, an additional chemical moiety is or comprises a ligand moiety for, e.g., cell receptors such as a sigma receptor, or an asialoglycoprotein receptor. In some embodiments, a ligand moiety is or comprises an anisamide moiety, which may be a ligand moiety for a sigma receptor.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, or multivalent fucose.

The ligand can be a substance, e.g., a drug, which can increase the uptake of the oligonucleotide agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to oligonucleotides described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that include a number of phosphorothioate linkages are also known to bind to serum protein, thus short oligonucleotides, e.g., oligonucleotides of about 5 bases, 10 bases, 15 bases, or 20 bases, including multiple of phosphorothioate linkages in the backbone are also amenable to oligonucleotides disclosed herein as ligands (e.g. as PK modulating ligands). In addition, aptamers that bind serum components (e.g. serum proteins) are also suitable for use as PK modulating ligands in the embodiments described herein.

In some embodiments, an additional chemical moiety conjugated to an oligonucleotide is capable of targeting the oligonucleotide to a cell in the central nervous system.

In some embodiments, an additional chemical moiety comprises or is a cell receptor ligand. In some embodiments, an additional chemical moiety comprises or is a protein binder, e.g., one binds to a cell surface protein. Such moieties among other things can be useful for targeted delivery of oligonucleotides to cells expressing the corresponding receptors or proteins. In some embodiments, an additional chemical moiety of a provided oligonucleotide comprises anisamide or a derivative or an analog thereof and is capable of targeting the oligonucleotide to a cell expressing a particular receptor, such as the sigma 1 receptor.

In some embodiments, a provided oligonucleotide is formulated for administration to a body cell and/or tissue expressing its target. In some embodiments, an additional chemical moiety conjugated to an oligonucleotide is capable of targeting the oligonucleotide to a cell.

In some embodiments, an additional chemical moiety on an oligonucleotide as disclosed herein is connected via a linker. Various linkers are available in the art and may be utilized in accordance with the present disclosure, for example, those utilized for conjugation of various moieties with proteins (e.g., with antibodies to form antibody-drug conjugates), nucleic acids, etc. Certain useful linkers are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, and/or WO 2021/071858, the linker moieties of each which are independently incorporated herein by reference. In some embodiments, a linker can connect two or more additional chemical moieties to an oligonucleotide chain as described herein. For example, some embodiments, one or two or three or more additional chemical moieties, e.g., GalNAc moieties, are connected to an oligonucleotide chain (e.g., at 5′-end) through a multivalent linker moiety.

In some embodiments, an additional chemical moiety is cleaved from the remainder of an oligonucleotide, e.g., an oligonucleotide chain, e.g., after administration to a system, cell, tissue, organ, subject, etc. In some embodiments, additional chemical moieties promote, increase, and/or accelerate delivery to certain cells, and after delivery of oligonucleotides into such cells, additional chemical moieties are cleaved from oligonucleotides. In some embodiments, a linker is as described in WO 2012/030683, WO 2021/030778, WO 2020/154344, WO 2020/154343, WO 2020/154342, WO 2020/165077, WO 2020/201406, WO 2020/216637, or WO 2020/252376.

### Lipid Conjugates

In some embodiments, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid-based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In some embodiments, the ligand is a moiety, e.g., a vitamin, which is taken up by a target cell, e.g., a proliferating cell. Exemplary vitamins include vitamin A, E, and K.

### Cell Permeation Agents

In some embodiments, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP. An RFGF analogue (e.g., amino acid sequence AALLPVLLAAP-containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ) and the Drosophila Antennapedia protein (RQIKIWFQNRRMKWKK) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to an oligonucleotide agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods described herein may be linear or cyclic, and may be modified, e.g., glycosylated or methylated, to facilitate targeting to a specific tissue(s). RGD-containing peptides and peptidomimetics may include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin, or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### Carbohydrate Conjugates

In some embodiments, oligonucleotides described herein further includes a carbohydrate. The carbohydrate conjugated oligonucleotide is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In some embodiments, a carbohydrate conjugate is a monosaccharide.

In some embodiments, the carbohydrate conjugate further includes one or more additional ligands as described above, such as, but not limited to, a PK modulator and/or a cell permeation peptide.

Additional carbohydrate conjugates (and linkers) suitable for use in the oligonucleotides described herein include those described in PCT Publication Nos. WO 2014/179620 and WO 2014/179627, the entire contents of each of which are incorporated herein by reference.

### Linkers

In some embodiments, the conjugate or ligand described herein can be attached to an oligonucleotide with various linkers.

Linkers typically include a direct bond or an atom such as oxygen or sulfur, a unit such as NR₈, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R₈), C(O), substituted or unsubstituted aryl, substituted or unsubstituted heteroaryl, substituted or unsubstituted heterocyclic; where R₈ is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

In one embodiment, oligonucleotides described herein are conjugated to a carbohydrate through a linker. Linkers include bivalent and trivalent branched linker groups. Exemplary oligonucleotide carbohydrate conjugates with linkers include, but are not limited to, those described in formulas 24-35 of PCT Publication No. WO 2018/195165.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

In certain instances, the oligonucleotide described herein can be modified by a non-ligand group. A number of non-ligand molecules have been conjugated to oligonucleotides in order to enhance the activity, cellular distribution, or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### Methods of Making the Oligonucleotides

The oligonucleotides described herein may be synthesized using an oligonucleotide that bears a pendant reactive functionality. This reactive oligonucleotide may be reacted directly with commercially-available ligands, ligands that are synthesized bearing any of a variety of protecting groups, or ligands that have a linking moiety attached thereto, so as to provide the GalNAc moiety onto the oligonucleotide.

The oligonucleotides described herein may be conveniently and routinely made through the well-known technique of solid-phase synthesis. Equipment for such synthesis is sold by several vendors including, for example, Applied Biosystems (Foster City, Calif.). Oligonucleotides described herein can be synthesized by standard methods known in the art as further discussed below, e.g., by use of an automated DNA synthesizer, such as are commercially available from, for example, Biosearch, Applied Biosystems, Inc. Any other means for such synthesis known in the art may additionally or alternatively be employed. It is also known to use similar techniques to prepare other oligonucleotides, such as the phosphorothioates and alkylated derivatives.

The oligonucleotides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides described herein are synthesized by an automated synthesizer using phosphoramidites derived from ligand-nucleoside conjugates in addition to the standard phosphoramidites and non-standard phosphoramidites that are commercially available and routinely used in oligonucleotide synthesis.

The oligonucleotides described herein are synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Representative U.S. patents that teach the preparation of the oligonucleotides include, but are not limited to, U.S. Pat. Nos. 5,034,506; 5,166,315; 5,185,444; 5,214,134; 5,216,141; 5,235,033; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

The oligonucleotide can be prepared using solution-phase or solid-phase organic synthesis or both. Organic synthesis offers the advantage that the oligonucleotide including unnatural or alternative nucleotides can be easily prepared. Single-stranded oligonucleotides described herein can be prepared using solution-phase or solid-phase organic synthesis or both.

It is contemplated that for any sequence identified herein, further optimization could be achieved by systematically either adding or removing linked nucleotides to generate longer or shorter sequences. Such optimized sequences can be adjusted by, e.g., the introduction of alternative nucleobases, alternative sugar moieties, and/or alternative internucleotide linkages as described herein or as known in the art, including alternative nucleobases, alternative sugar moieties, and/or alternative internucleotide linkages as known in the art and/or discussed herein to further optimize the molecule (e.g., increasing serum stability or circulating half-life, increasing thermal stability, enhancing transmembrane delivery, targeting to a particular location or cell type, and/or increasing interaction with RNA editing enzymes (e.g., ADAR)).

### Methods of Use

Oligonucleotides modified to include a GalNAc moiety as disclosed herein (e.g., a structure of any of Formula (I), (II), (III), (IV), or (IV)) can be delivered to a cell for a therapeutic or diagnostic purpose. The specific features of the oligonucleotide can deem is suitable for any number of end-uses. Exemplary oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, adaptors, triplex-forming oligonucleotides, G-quadruplex oligonucleotides, RNA activators, immune-stimulatory oligonucleotides, and decoy oligonucleotides. Choice of features of the oligonucleotides (beyond the presence of one or more GalNAc modifications as disclosed herein) is within the skill of the artisan, in view of the intended activity and use of the oligonucleotide.

Stem loop structures can act as a recruitment domain for the ADAR enzyme (e.g., an ADAR-recruiting domain), yet the oligonucleotides as disclosed herein can affect ADAR recruitment and activity against a target adenosine in a target RNA without such a stem loop structure. Thus, in some embodiments, the oligonucleotides disclosed herein do not include a stem-loop structure. Alternatively, in some embodiments, the oligonucleotides disclosed herein do include a stem-loop structure.

In some embodiments, the oligonucleotides described herein may further include a 5′ cap structure. In some embodiments, the 5′ cap structure is a 2,2,7-trimethylguanosine cap.

In some cases, at least one nucleobase is a hypoxanthine or a 5′-methylcytosine. In some cases, the modified oligonucleotide has a sequence that is sufficiently complementary to a target RNA. In some cases, the modified oligonucleotide is capable of binding and recruiting an ADAR enzyme to perform editing on a target adenosine of the target RNA. In some cases, the target RNA is SERPINA1, UGP2, LRRK2, NAV 1.7, NRF2 or TDP43.

### Therapeutic Uses

Provided herein are uses of an oligonucleotide described herein as a therapeutic agent. In various embodiments, the oligonucleotide is a structural gene, a gene including control and termination regions, a self-replicating system such as a viral or plasmid DNA, a single-stranded or double-stranded RNAi agent, shRNA, an antisense oligonucleotide, a ribozyme, a microRNA, a microRNA mimics, a supermir, an aptamer, an antimir, an antagomir, an adaptor, a triplex-forming oligonucleotide, a G-quadruplex oligonucleotide, an RNA activator, an immuno-stimulatory oligonucleotide, or a decoy oligonucleotide.

In some instances, provided are methods of treating a disease or disorder in a patient in need thereof, comprising administering to the patient a therapeutically effective amount of an oligonucleotide disclosed herein. The term “therapeutically effective amount” refers to an amount effective in treating and/or ameliorating a disease or disorder in a patient. Further provided are methods of delivering an oligonucleotide disclosed herein to a cell comprising contacting the cell with the oligonucleotide or a pharmaceutical composition thereof as disclosed herein. In some implementations, the cell can be contacted in vitro. In other implementations, the cell is contacted in vivo. In some cases, the patient is a mammalian subject. A mammalian subject may include but is not limited to a human or a mouse subject. In yet other implementations wherein the cell is contacted ex vivo, the cell is obtained from a human or mouse subject. In some cases, the cell is a tumor cell. In some cases, the cell is a muscle cell.

In some implementations, the oligonucleotides are administered to the patient to treat a disease or disorder. Non-limiting examples of diseases or disorders which may be treated with the oligonucleotides disclosed herein include cancer, infectious diseases, autoimmune disorders, and neurological disorders. In certain implementations, the oligonucleotides disclosed herein are used as a vaccine. Genetic vaccination, or the administration of nucleic acid molecules (e.g., RNA) to a patient and subsequent transcription and/or translation of the encoded genetic information, is useful in the treatment and/or the prevention of inherited genetic diseases but also autoimmune diseases, infectious diseases, cancerous or tumor-related diseases as well as inflammatory diseases. Genetic vaccination is particularly use in the treatment of cancer because cancer cells express antigens, tumors are generally not readily recognized and eliminated by the host, as evidenced by the development of disease. Non-limiting examples of specific diseases and disorders which are contemplated to be treated with the oligonucleotides disclosed herein include cystic fibrosis, albinism, alpha-1-antitrypsin deficiency, Alzheimer disease, amyotrophic lateral sclerosis, asthma, 11-thalassemia, Cadasil syndrome, Charcot-Marie-Tooth disease, chronic obstructive pulmonary disease, distal spinal muscular atrophy, Duchenne/Becker muscular, dystrophy, dystrophic epidermolysis bullosa, epidermylosis bullosa, Fabry disease, Factor V Leiden associated disorders, familial adenomatous, polyposis, galactosemia, Gaucher's disease, glucose-6-phosphate dehydrogenase deficiency, hemophilia, hereditary hematochromatosis, Hunter syndrome, Huntington's disease, Hurler syndrome, inflammatory bowel disease, inherited polyagglutination syndrome, Leber congenital amaurosis, Lesch-Nyhan syndrome, Lynch syndrome, Marfan syndrome, mucopolysaccharidosis, muscular dystrophy, myotonic dystrophy types I and II, neurofibromatosis, Niemann-Pick disease type A, B and C, NY-ESO-1 related cancer, Parkinson's disease, Peutz-Jeghers syndrome, phenylketonuria, Pompe's disease, primary ciliary disease, prothrombin mutation related disorders, pulmonary hypertension, retinitis pigmentosa, Sandhoff disease, severe combined immune deficiency syndrome, sickle cell anemia, spinal muscular atrophy, Stargardt's Disease, Tay-Sachs disease, Usher syndrome, X-linked immunodeficiency, Sturge-Weber syndrome, Rett syndrome, and cancer.

Vaccines. The oligonucleotides of the disclosure are useful as vaccines, in which the oligonucleotide is a DNA or an RNA that may encode an immunogen, antigen or neoantigen. The immune system of a host provides a means for quickly and specifically mounting a protective response to pathogenic microorganisms and also for contributing to rejection of malignant tumors. Immune responses have been generally described as including humoral responses, in which antibodies specific for antigens are produced by differentiated B lymphocytes, and cell mediated responses, in which various types of T lymphocytes eliminate antigens by a variety of mechanisms. For example, CD4 (also called CD4+) helper T cells that are capable of recognizing specific antigens may respond by releasing soluble mediators such as cytokines to recruit additional cells of the immune system to participate in an immune response. CD8 (also called CD8+) cytotoxic T cells are also capable of recognizing specific antigens and may bind to and destroy or damage an antigen-bearing cell or particle. In particular, cell mediated immune responses that include a cytotoxic T lymphocyte (CTL) response can be important for elimination of tumor cells and cells infected by a microorganism, such as virus, bacteria, or parasite.

Thus, the disclosure includes methods for inducing an immune response in a patient in need thereof, comprising administering to the patient a therapeutically effective amount of the oligonucleotide (e.g., formulated as an antigenic composition) of the disclosure. In some implementations, the administering is by intramuscular, intratumoral, intravenous, intraperitoneal, or subcutaneous delivery.

In various implementations, administering the oligonucleotides of the disclosure (e.g., formulated as a composition, pharmaceutical formulation, or antigenic composition) to a subject can result in an increase in the amount of antibodies (e.g., neutralizing antibodies) against the antigen that is produced in the subject relative to the amount of antibodies that is produced in a subject who was not administered the oligonucleotides. In some implementations, the increase is a 2-fold increase, a 5-fold increase, a 10-fold increase, a 50-fold increase, a 100-fold increase, a 200-fold increase, a 500-fold increase, a 700-fold increase, or a 1000-fold increase.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the oligonucleotide that encodes an antigen as disclosed herein generates an immune response that recognizes, and preferably ameliorates and/or neutralizes, an infection as described herein. Methods for assessing antibody responses after administration of an antigenic composition (immunization or vaccination) are known in the art and/or described herein. In some implementations, the immune response comprises a T cell-mediated response (e.g., peptide-specific response such as a proliferative response or a cytokine response). In some implementations, the immune response comprises both a B cell and a T cell response. Antigenic compositions can be administered in a number of suitable ways, such as intramuscular injection, intratumoral injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Cancer. Various cancers (e.g., cervical cancer) may be treated with the oligonucleotides of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

Infectious Diseases. Also disclosed herein are methods of treating an infectious disease (e.g., a bacterial, viral, fungal, or parasitic infection) in a patient in need thereof, comprising administering to the patient an effective amount of an oligonucleotide of the disclosure. Non-limiting examples of infectious diseases include hepatitis (such as HBV infection or HCV infection), RSV, influenza, adenovirus, rhinovirus, or other viral infections. In some implementations, the administering is by intramuscular, intratumoral, intravenous, intraperitoneal, or subcutaneous delivery.

Autoimmune diseases. Various autoimmune diseases and autoimmune-related diseases may be treated with the oligonucleotides of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed ryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia**, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & Ill autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA). In some implementations, the administering is by intramuscular, intratumoral, intravenous, intraperitoneal, or subcutaneous delivery.

Neurological diseases. Various neurological diseases may be treated with the oligonucleotides of the present disclosure. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy—Congenital, Myopathy—Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Post infectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Shortlasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy. In some implementations, the administering is by intramuscular, intratumoral, intravenous, intraperitoneal, or subcutaneous delivery.

In some cases, the oligonucleotides disclosed herein have a sequence that is sufficiently complementary to a target RNA. The target RNA can be associated with a disease or disorder contemplated to be treated with the oligonucleotides of the disclosure. In some cases, the target RNA is selected from Serpina1, LRRK2, NRF2, TDP-43, Nav1.7, and PCKS9.

### ADAR Editing

Adenosine deaminases acting on RNA (ADAR) are enzymes which bind to double-stranded RNA (dsRNA) and convert adenosine to inosine through deamination. In RNA, inosine functions similarly to guanosine for translation and replication. Thus, conversion of adenosine to inosine in an mRNA can result in a codon change that may lead to changes to the encoded protein and its functions. There are three known ADAR proteins expressed in humans, ADAR1, ADAR2, and ADAR3. ADAR1 and ADAR2 are expressed throughout the body whereas ADAR3 is expressed only in the brain. ADAR1 and ADAR2 are catalytically active, while ADAR3 is thought to be inactive.

Synthetic single-stranded oligonucleotides have been shown capable of utilizing the ADAR proteins to edit target RNAs by deaminating particular adenosines in the target RNA. The oligonucleotides are complementary to the target RNA with the exception of at least one mismatch opposite the adenosine to be deaminated. However, the previously disclosed methods have not been shown to have the required selectivity and/or stability to allow for their use as therapies. Accordingly, new oligonucleotides capable of utilizing the ADAR proteins to selectively edit target RNAs in a therapeutically effective manner are needed.

In certain embodiments, the oligonucleotides described herein are complementary to target RNA and are capable of recruiting ADAR enzymes used to edit a target nucleobase on the target RNA, e.g., to deaminate a target adenosine on the target RNA. In some embodiments, only one nucleobase (e.g., one adenosine) is edited (e.g., deaminated). In some embodiments, 1, 2, or 3 nucleobases are edited. In some embodiments, the oligonucleotide includes at least one mismatch, wobble, insertion or deletion. In some cases, the oligonucleotide includes a mismatch opposite the target nucleobase. The oligonucleotides described herein may further include modifications (e.g., alternative nucleotides) to increase stability and/or increase deamination efficiency. In some embodiments, the oligonucleotides described herein comprises 1, 2, 3, 4, or 5 mismatches or wobbles or insertions or deletions (or any combination thereof).

The disclosure therefore contemplates the use of the disclosed oligonucleotides for use in a method for making a change in a target RNA sequence in a mammalian, preferably human cell, as described herein. Similarly, the disclosure provides the use of these oligonucleotides in the manufacture of a medicament for making a change in a target RNA sequence in a mammalian, preferably human cell, as described herein. In some embodiments, the target RNA is a mRNA.

The disclosure also relates to a method for the deamination of at least one specific target adenosine present in a target RNA sequence in a cell, said method including the steps of: providing said cell with an oligonucleotide described herein; allowing uptake by the cell of the oligonucleotide; allowing annealing of the oligonucleotide to the target RNA sequence; allowing a mammalian ADAR enzyme to deaminate said target adenosine in the target RNA sequence to an inosine; and optionally identifying the presence of the inosine in the RNA sequence.

In some embodiments, provided herein are oligonucleotides, compositions, and methods wherein two adenosines that are next to each other are co-deaminated by an RNA editing enzyme such as ADAR. In this particular case, the UAA stop codon is converted into a UII Trp-encoding codon. Other examples of modifications resulting from deamination of target adenosines within a target codon are provided in Tables 2 and 3 below.

TABLE 2

| Target | Amino Acid Encoded | Modified | Amino Acid Encoded by |
|---|---|---|---|
| Codon | by Target Codon | Codon | Modified Codon |
| AAA | Lys | IAA | Glu |
| | | AIA | Arg |
| | | IIA | Gly |
| | | AII | Arg |
| | | IAI | Glu |
| | | III | Gly |
| AAC | Asn | IAC | Asp |
| | | AIC | Ser |
| | | IIC | Gly |
| AAG | Lys | IAG | Glu |
| | | AIG | Arg |
| | | IIG | Gly |
| AAU | Arg | IAU | Asp |
| | | AIU | Ser |
| | | IIU | Gly |
| ACA | Thr | ICA | Ala |
| | | ICI | Ala |
| ACC | Thr | ICC | Ala |
| ACG | Thr | ICG | Ala |
| ACU | Thr | ICU | Ala |
| AGA | Arg | IGA | Gly |
| | | IGI | Gly |
| AGC | Ser | IGC | Gly |
| AGG | Arg | IGG | Gly |
| AGU | Ser | IGU | Gly |
| AUA | Ile | IUA | Asp |
| | | AUI | Met |
| | | IUI | Val |
| AUC | Ile | IUC | Val |
| AUG | Met | IUG | Val |
| AUU | Ile | IUU | Val |
| CAA | Gln | CIA | Arg |
| | | CII | Arg |
| CAC | His | CIC | Arg |
| CAG | Gln | CIG | Arg |
| CAU | His | CIU | Arg |
| GAA | Glu | GIA | Gly |
| | | GII | Gly |
| GAC | Asp | GIC | Gly |
| GAG | Glu | GIG | Gly |
| GAU | Asp | GIU | Gly |
| UAA | Stop | UII | Trp |
| UGA | Stop | UGI | Trp |
| UAC | Tyr | UIC | Cys |
| UAG | Stop | UIG | Trp |
| UAU | Tyr | UIU | Cys |

TABLE 3
Triplet Base Composition and Resulting Edited Triplet

| Target Codon | Modified Codon |
|---|---|
| AAA | AIA |
| AAC | AIC |
| AAG | AIG |
| AAU | AIU |
| CAA | CIA |
| CAC | CIC |
| CAG | CIG |
| CAU | CIU |
| GAA | GIA |
| GAC | GIC |
| GAG | GIG |
| GAU | GIU |
| UAA | UIA |
| UAC | UIC |
| UAG | UIG |
| UAU | UIU |

Because the deamination of the adenosine to an inosine may result in a protein that is no longer suffering from the mutated A at the target position, the identification of the deamination into inosine may be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. When the presence of a target adenosine causes aberrant splicing, the read-out may be the assessment of whether the aberrant splicing is still taking place, or not, or taking place less often. On the other hand, when the deamination of a target adenosine is wanted to introduce a splice site, then similar approaches can be used to check whether the required type of splicing is indeed taking place. A suitable manner to identify the presence of an inosine after deamination of the target adenosine is RT-PCR and sequencing, using methods that are well-known to the person skilled in the art.

In general, mutations in any target RNA that can be reversed using oligonucleotides disclosed herein are G-to-A mutations, and oligonucleotide constructs can be designed accordingly. Mutations that may be targeted using oligonucleotide constructs also include C to A, U to A (T to A on the DNA level) in the case of recruiting adenosine deaminases. Although RNA editing in the latter circumstances may not necessarily revert the mutation to wild-type, the edited nucleotide may give rise to an improvement over the original mutation. For example, a mutation that causes an in frame stop codon—giving rise to a truncated protein, upon translation—may be changed into a codon coding for an amino acid that may not be the original amino acid in that position, but that gives rise to a (full length) protein with at least some functionality, at least more functionality than the truncated protein.

The oligonucleotides described herein particularly suitable for treating genetic diseases, such as cystic fibrosis, albinism, alpha-1-antitrypsin (A1AT) deficiency, Alzheimer disease, amyotrophic lateral sclerosis, asthma, 11-thalassemia, Cadasil syndrome, Charcot-Marie-Tooth disease, chronic obstructive pulmonary disease (COPD), distal spinal muscular atrophy (DSMA), Duchenne/Becker muscular dystrophy, dystrophic epidermolysis bullosa, epidermylosis bullosa, Fabry disease, Factor V Leiden associated disorders, familial adenomatous, polyposis, galactosemia, Gaucher's disease, glucose-6-phosphate dehydrogenase deficiency, haemophilia, hereditary hematochromatosis, Hunter syndrome, Huntington's disease, Hurler syndrome, inflammatory bowel disease (IBD), inherited polyagglutination syndrome, Leber congenital amaurosis, Lesch-Nyhan syndrome, Lynch syndrome, Marfan syndrome, mucopolysaccharidosis, muscular dystrophy, myotonic dystrophy types I and II, neurofibromatosis, Niemann-Pick disease type A, B and C, NY-ESO-1 related cancer, Parkinson's disease, Peutz-Jeghers syndrome, phenylketonuria, Pompe's disease, primary ciliary disease, prothrombin mutation related disorders (e.g., prothrombin G20210A mutation), pulmonary hypertension, retinitis pigmentosa, Sandhoff disease, severe combined immune deficiency syndrome (SCID), sickle cell anemia, spinal muscular atrophy, Stargardt's disease, Tay-Sachs disease, Usher syndrome, X-linked immunodeficiency, Sturge-Weber syndrome, Rett syndrome, and various forms of cancer (e.g. BRCA1 and 2 linked breast cancer and ovarian cancer).

Oligonucleotides described herein may deaminate the adenosine mutation resulting in an increase in protein activity.

In certain embodiments, treatment is performed on a subject who has been diagnosed with a mutation in a gene, but does not yet have disease symptoms (e.g., an infant such as a subject that is 1 month to 12 months old or subject under the age of 2). In other embodiments, treatment is performed on an individual who has at least one symptom.

Treatment may be performed in a subject of any age, starting from infancy to adulthood. Subjects may begin treatment, for example, at birth, six months, or 1, 2, 3, 4, 5, 6, 7, 8, 9, 10, 11, 12, 13, 15, or 18 years of age.

In certain embodiments, the oligonucleotide increases (e.g., an increase by 10%, 20%, 30%, 40%, 50%, 60%, 70%, 80%, 90%, 100%, 150%, 200%, 300%, 400%, 500%, 600%, 700%, 800%, 900%, 1000% or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) protein activity in vitro and/or in vivo.

In some embodiments, the oligonucleotide increases (e.g., an increase by 10%, 20%, 30%, 40%, 50%, 60%, 70%, 80%, 90%, 100%, 150%, 200%, 300%, 400%, 500%, 600%, 700%, 800%, 900%, 1000% or more, or an increase by more than 1.2-fold, 1.4-fold, 1.5-fold, 1.8-fold, 2.0-fold, 3.0-fold, 3.5-fold, 4.5-fold, 5.0-fold, 10-fold, 15-fold, 20-fold, 30-fold, 40-fold, 50-fold, 100-fold, 1000-fold, or more) protein activity in the brain.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Pharmaceutical Compositions and Routes of Administration

i. Lipid-Free Delivery Methods

Delivery of therapeutics to the liver as lipid nanoparticles (LNPs) has emerged as a popular strategy. In these prior delivery methods, accumulation of apolipoprotein E (ApoE) on the surface is thought to lead to uptake primarily by hepatocytes via low-density lipoprotein receptor (LDLR)-mediated endocytosis.

Oligonucleotides described herein can advantageously be delivered without the use of lipid-based carriers or vehicles. Without wishing to be bound by any particular theory, it is thought that conjugation of oligonucleotides to GalNAc moieties enables the oligonucleotides to be delivered to cellular targets via the asialoglycoprotein receptor (ASGPR) pathway. GalNAc-modified oligonucleotides delivered through such lipid-free methods benefit from several advantages over prior methods (e.g., LNP-based methods). Exploitation of the ASGPR pathway is thought to be favorable because the receptor is highly expressed in the liver but not other tissues, this process leads to rapid endocytosis of the medicine when bound by GalNAc, and ASGPR is rapidly recycled to the hepatocyte surface. See e.g., Kasiewicz, L. N., et al., Nat Commun (2023), 14, 2776.

The oligonucleotides described herein are preferably formulated into pharmaceutical compositions for administration to patients (e.g., human patients) in a biologically compatible form suitable for administration in vivo. The presence of a GalNAc moiety on the oligonucleotides disclosed herein allows for administration without the need of a lipid nanoparticle or liposomal delivery. Instead, the GalNAc can bind to ASGPR and deliver the oligonucleotide across a cell membrane.

The oligonucleotides described herein may be administered, for example, by oral, parenteral, intrathecal, intracerebroventricular, intraparenchymal, buccal, sublingual, nasal, rectal, patch, pump, intratumoral, or transdermal administration and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration. Parenteral administration may be by continuous infusion over a selected period of time.

An oligonucleotide described herein may be orally administered, for example, with an inert diluent or with an assimilable edible carrier, or it may be enclosed in hard- or soft-shell gelatin capsules, or it may be compressed into tablets, or it may be incorporated directly with the food of the diet. For oral therapeutic administration, an oligonucleotide described herein may be incorporated with an excipient and used in the form of ingestible tablets, buccal tablets, troches, capsules, elixirs, suspensions, syrups, and wafers. An oligonucleotide described herein may also be administered parenterally. Solutions of an oligonucleotide described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. An oligonucleotide described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate.

The oligonucleotides described herein may be administered to an animal, e.g., a human, alone or in combination with pharmaceutically acceptable carriers, as noted herein, the proportion of which is determined by the solubility and chemical nature of the oligonucleotide, chosen route of administration, and standard pharmaceutical practice.

ii. Membranous Molecular Assembly Delivery Methods

While oligonucleotides described herein are suitable for delivery and are preferably delivered without liposomal or lipid-based systems, the oligonucleotides can be used with such systems if desired.

Oligonucleotides described herein can be delivered using a variety of membranous molecular assembly delivery methods including polymeric, biodegradable microparticle, or microcapsule delivery devices known in the art. For example, a colloidal dispersion system may be used for targeted delivery an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA and can mediate RNase H-mediated gene silencing. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable oligonucleotide delivery vehicles, incorporating an oligonucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169. These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems including non-ionic surfactant and cholesterol. Non-ionic liposomal formulations including NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P. Pharma. Sci., 4(6):466).

Liposomes may also be sterically stabilized liposomes, including one or more specialized lipids that result in enhanced circulation lifetimes relative to liposomes lacking such specialized lipids. Examples of sterically stabilized liposomes are those in which part of the vesicle-forming lipid portion of the liposome (A) includes one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes including one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes including (1) sphingomyelin and (2) the ganglioside G_{M1} or a galactocerebroside sulfate ester. U.S. Pat. No. 5,543,152 (Webb et al.) discloses liposomes including sphingomyelin. Liposomes including 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotides (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analog, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that include positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotides into the skin. In some implementations, liposomes are used for delivering oligonucleotides to epidermal cells and also to enhance the penetration of oligonucleotides into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems including non-ionic surfactant and cholesterol. Non-ionic liposomal formulations including Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with oligonucleotide are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the disclosed oligonucleotides and methods are described in WO 2009/086558, and WO 2009/088891. WO 2008/042973 also describes formulations that are amenable to the present oligonucleotides and methods.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotide for use in the methods described herein can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

iii. Lipid Nanoparticle-Based Delivery Methods

As discussed above, the oligonucleotides described herein are suitable for delivery and are preferably delivered without liposomal or lipid-based systems. However, the oligonucleotides can be used with such systems if desired.

Oligonucleotides described herein may be fully encapsulated in a lipid formulation, e.g., a lipid nanoparticle (LNP), or other nucleic acid-lipid particle. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to oligonucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part described herein.

Non-limiting examples of cationic lipid include N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can include, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

### Dosages

The dosage of the compositions (e.g., a composition including an oligonucleotide) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compositions described herein may be administered initially in a suitable dosage that may be adjusted as required, depending on the clinical response. In some embodiments, the dosage of a composition (e.g., a composition including an oligonucleotide) is a prophylactically or a therapeutically effective amount.

### Kit

Provided herein are kits including (a) a pharmaceutical composition including an oligonucleotide that results in deamination of an adenosine in an mRNA in a cell or subject described herein, and (b) a package insert with instructions to perform any of the methods described herein. In some embodiments, the kit includes (a) a pharmaceutical composition including an oligonucleotide that results in deamination of an adenosine in an mRNA in a cell or subject described herein, (b) an additional therapeutic agent, and (c) a package insert with instructions to perform any of the methods described herein.

### EXAMPLES

### General Methods

All guide oligonucleotides were chemically synthesized on an automated RNA/DNA synthesizer using standard β-cyanoethylphosphoramidite chemistry and a universal solid support such as controlled pore glass (CPG). Phosphoramidites of N-protected β-homo-DNA was synthesized utilizing reported procedures. See Matheus Froeyen et al., (2001) Chem. Eur. J., 7: 5183-5794, Herdewijn, (2010) Chem. Biodivers., 7: 1-59, Jabgunde et al., (2019) Tetrahedron, 75: 1107-1114. Other 5′-O-DMT-3′-phosphoramidite RNA, 2′-O-methyl-RNA and DNA monomers, i.e., A, C, G, U, and T, were purchased from commercial sources. After synthesis, oligonucleotides were cleaved from the solid support, deprotected, and purified by a HPLC system using standard protocols. Oligonucleotides were desalted, dialyzed, and lyophilized. The purity of each lyophilized oligo was >80% as determined by analytical reversed-phase HPLC. The sequence integrity of the oligonucleotides was determined by ESI-MS. (The sequences of the various oligonucleotides are provided herein in Tables 5 and 6).

Human ADAR2 sequence (NM_001112.4) was cloned into pcDNA3.1 plasmid under the control of the CMV promoter using BamHI and XbaI restriction sites (Quintara Bio, Berkeley, CA) and the correct insert was sequence verified. This plasmid henceforth will be denoted as ADAR2/pcDNA3.1. For editing experiments, 2 μg of ADAR2/pcDNA3.1 plasmid were transfected into 5×10⁶ HEK293T cells (ATCC) using 25 μL of Lipofectamine 3000 and 24 μL of P3000 (Life Technologies) per 10 cm dish. After 4 hours, the culture media was replenished with fresh warmed media (DMEM High Glucose; Life Technologies). 12-16 hours after transfection, the transfected HEK293T cells were transfected with guide oligonucleotides such that the final concentration in the each well was 100 nM. All transfections were carried out with Lipofectamine 3000 (0.4 μL/per well) in a 96-well format, according to manufacturer's instructions. 12-16 hours after the second transfection, the cells were washed once with ice cold PBS and total mRNA isolation was performed using Dyna Beads mRNA Direct Kit (Life Technologies) adapted for KingFisher Flex Purification (Life Technologies), according to manufacturer's instructions. The samples were treated with TURBO DNase (Life Technologies) prior to elution. The resultant isolated mRNA was used for cDNA synthesis using SuperScript IV Vilo according to the manufacturer's instructions (Life Technologies). One μl of the cDNA was used as template for PCR (Platinum II Hot-Start PCR Master Mix; Life Technologies) using gene specific primers to generate an amplicon for Sanger sequencing. Sanger sequencing was performed by Quintara Biosciences (Berkeley, CA). Adenosine to guanosine editing yields were quantified by measuring the peak height of adenosine and guanosine and dividing the guanosine peak height by the total peak height measurements of adenosine and guanosine combined.

### Example 1: Synthesis of Oligonucleotides

Oligonucleotides of the disclosure were synthesized using standard solid phase phosphoramidite chemistry in 200 nM-25 μM scale as described below. Monomer amidites were dissolved in anhydrous acetonitrile at 0.05M. They were delivered to a solid-phase synthesis column with 0.25M 5-(Ethylthio)-1H-tetrazole (ETT) in acetonitrile. Two couplings of 6 minutes each were carried out. The optimized solid phase synthesis procedures as example for 1, 10, and 25 μmole scale syntheses are summarized in Table 4.

TABLE 4
Solid phase synthesis conditions.

| 1 μmole synthesis protocol | 10 μmole synthesis protocol | 25 μmole synthesis protocol |
|---|---|---|
| Initialization | Initialization | Initialization |
| Wash 225 μl, repeat x2 | Wash 1500 μL, repeat x2 | Wash 3000 μL, repeat x2 |
| Synthesis Cycle 1-1 | Synthesis Cycle 1-1 | Synthesis Cycle 1-1 |
| De-Blocking 200 μL, 1 min, | De-Blocking 1000 μL, 1 min, | De-Blocking 2000 μL, 1 min, |
| repeat x3 | repeat x3 | repeat x3 |
| Wash 225 μL, repeat x3 | Wash 1500 μL, repeat x3 | Wash 3000 μL, repeat x3 |
| Coupling: ACT 110 μL + Amidite | Coupling: ACT 460 μL + Amidite | Coupling: ACT 690 μL + Amidite |
| 90 μL; DNA 3 min, RNA 6 min, | 400 μL; DNA 3 min, RNA 6 min, | 600 μL; DNA 3 min, RNA 6 min, |
| repeat x3 | repeat x3 | repeat x3 |
| Wash 225 μL, repeat x2 | Wash 1500 μL, repeat x2 | Wash 3000 μL, repeat x2 |
| Ox 200 μL, 1 min | Ox 1000 μL, 1 min | Ox 2000 μL, 1 min |
| Sul 200 μL, 5 min, repeat x2 | Sul 1000 μL, 5 min, repeat x2 | Sul 2000 μL, 5 min, repeat x2 |
| PA 200 μL, 20 min, repeat x3 | PA 1000 μL, 20 min, repeat x3 | PA 2000 μL, 20 min, repeat x3 |
| Wash 225 μL | Wash 1500 μL | Wash 3000 μL |
| Caping: CapA 100 μL + CapB | Caping: CapA 400 μL + CapB | Caping: CapA 750 μL + CapB |
| 100 μL, 60 second | 400 μL, 60 second | 750 μL, 60 second |
| Wash 225 μL | Wash 1500 μL | Wash 3000 μL |
| Synthesis Cycle 2-end | Synthesis Cycle 2-end | Synthesis Cycle 2-end |
| De-Blocking 200 μL, 1 min, | De-Blocking 1200 μL, 1 min, | De-Blocking 2500 μL, 1 min, |
| repeat x2 | repeat x2 | repeat x2 |
| Wash 225 μL, repeat x3 | Wash 1500 μL, repeat x3 | Wash 3000 μL, repeat x3 |
| Coupling: ACT 90 μL + Amidite | Coupling: ACT 460 μL + Amidite | Coupling: ACT 690 μL + Amidite |
| 110 μL; DNA 3 min, RNA 6 min, | 400 μL; DNA 3 min, RNA 6 min, | 600 μL; DNA 3 min, RNA 6 min, |
| repeat x2 | repeat x2 | repeat x2 |
| Wash 225 μL, repeat x2 | Wash 1500 μL, repeat x2 | Wash 3000 μL, repeat x2 |
| Ox 200 μL, 1 min | Ox 1000 μL, 1 min | Ox 2000 μL, 1 min |
| Sul 200 μL, 5 min, repeat x2 | Sul 1000 μL, 5 min, repeat x2 | Sul 2000 μL, 5 min, repeat x2 |
| PA 200 μL, 20 min, repeat x3 | PA 1000 μL, 20 min, repeat x3 | PA 2000 μL, 20 min, repeat x3 |
| Wash 225 μL | Wash 1500 μL | Wash 3000 μL |
| Caping: CapA 100 μL + CapB | Caping: CapA 400 μL + CapB | Caping: CapA 750 μL + CapB |
| 100 μL, 60 second | 400 μL, 60 second | 750 μL, 60 second |
| Wash 225 μL | Wash 1500 μL | Wash 3000 μL |
| post synthesis | post synthesis | post synthesis |
| Wash 225 μL | Wash 1500 μL, repeat x1 | Wash 3000 μL, repeat x1 |
| De-Blocking 200 μL, 1 min, | De-Blocking 1200 μL, 1 min, | De-Blocking 2400 μL, 1 min, |
| repeat x2 | repeat x2 | repeat x2 |
| DEA wash: 200 μL, 3 min, | DEA wash: 1500 μL, 3 min, | DEA wash: 3000 μL, 3 min, |
| repeat x3 | repeat x3 | repeat x3 |
| Wash 225 μL, repeat x3 | Wash 1500 μL, repeat x3 | Wash 3000 μL, repeat x3 |

After completion of synthesis, the solid support was dried under vacuum and the oligonucleotide-bound cleaved from support with concentrated aqueous ammonia at 55° C. for 16 hours. The ammonia was removed under reduced pressure. The products adsorbed on the support were reconstituted in water and separated from the solid support using filtration and analyzed by LC/MS. The crude solution was purified by preparative IEx-HPLC and desalted. HELMs sequences of the synthesized oligomers are presented in Table 5, and nucleotide sequences are presented in Table 6.

TABLE 5
Synthesized Sequences

| Oligo | |
|---|---|
| No. | Sequences (HELM) |
| A1 | {m(C)[msPA].m(C)[sP].m(C)[sP].f(A)[sP].f(G)[sP].[moe]([m5C])P.[moe](A)P.f(G) |
| | [P].f(C)[sP].[moe](T)P.f(U)[msPA].m(C)P.f(A)[sP].m(G)[msPA].f(U)P.m(C)[sP].f |
| | (C)[sP].f(C)[sP].f(U)[sP].[moe](T)P.[moe](T)P.f(C)[sP].d(T)[sP].[Dh](C)[sP].d |
| | (I)[msPA].m(U)P.f(C)[sP].m(G)[sP].m(A)[msPA].m(U)} (SEQ ID NO: 1) |
| A2 | {m(C)[msPA].m(C)[sP].m(C)[sP].f(A)[sP].f(G)[sP].[moe]([m5C])P.[moe](A)P.f(G) |
| | [sP].f(C)[sP].[moe](T)P.f(U)[msPA].m(C)P.f(A)[sP].m(G)[msPA].f(U)P.m(C)[sP].f |
| | (C)[sP].f(C)[sP].f(U)[sP].[moe](T)P.[moe](T)P.f(C)[sP].d(T)[sP].[Dh](C)[sP].d |
| | (I)[msPA].m(U)P.f(C)[sP].m(G)[sP].m(A)[msPA].m(U)} (SEQ ID NO: 2) |
| A3 | {m(C)[msPA].m(C)[sP].m(C)[sP].f(A)[P].f(G)[sP].[moe]([m5C])P.[moe](A)P.f(G) |
| | [sP].f(C)[sP].[moe](T)P.f(U)[msPA].m(C)P.f(A)[sP].m(G)[msPA].f(U)P.m(C)[sP].f |
| | (C)[sP].f(C)[sP].f(U)[sP].[moe](T)P.[moe](T)P.f(C)[sP].d(T)[sP].[Dh](C)[sP].d |
| | (I)[msPA].m(U)P.f(C)[sP].m(G)[sP].m(A)[msPA].m(U)} (SEQ ID NO: 3) |
| A4 | {m(C)[msPA].m(C)[sP].m(C)[sP].f(A)[sP].f(G)[sP].[moe]([m5C])P.[moe](A)P.f(G) |
| | [sP].f(C)[sP].[moe](T)P.f(U)[msPA].m(C)P.f(A)[sP].m(G)[msPA].f(U)P.m(C)[sP].f |
| | (C)[sP].f(C)[sP].f(U)[sP].[moe](T)P.[moe](T)P.f(C)[sP].d(T)[sP].[Dh](C)[sP].d |
| | (I)[msPA].m(U)P.f(C)[sP].m(G)[sP].m(A)[msPA].m(U)} (SEQ ID NO: 4) |
| A5 | {m(C)[msPA].f(A)[sP].m(U)[sP].f(A)[sP].f(A)[sP].m(U)[sP].m(U)[sP].m(U)[sP].f |
| | (A)[sP].m(C)[sP].f(A)[msPA].m(C)[sP].f(A)[P].f(G)[msPA].f(A)[sP].f(A)[sP].f |
| | (G)[sP].m(C)[sP].f(A)[sP].f(A)[sP].m(U)[sP].f(G)[sP].d(C)[sP].[Dh](C)[sP].d |
| | (I)[msPA].m(U)[sP].f(C)[sP].m(A)[sP].m(C)[msPA].m(C)} (SEQ ID NO: 5) |
| A6 | {m(C)[msPA].f(A)[sP].m(U)[sP].f(A)[sP].f(A)[sP].m(U)[sP].m(U)[sP].m(U)[sP].f |
| | (A)[sP].m(C)[sP].f(A)[msPA].m(C)[sP].f(A)[sP].f(G)[msPA].f(A)[sP].f(A)[sP].f |
| | (G)[sP].m(C)[sP].f(A)[sP].f(A)[sP].m(U)[sP].f(G)[sP].d(C)[sP].[Dh](C)[sP].d |
| | (I)[msPA].m(U)[sP].f(C)[sP].m(A)[sP].m(C)[msPA].m(C)} (SEQ ID NO: 6) |
| A7 | m(C)[msPA].f(A)[sP].m(U)[sP].f(A)[sP].f(A)[P].m(U)[sP].m(U)[sP].m(U)[sP].f(A) |
| | [sP].m(C)[sP].f(A)[msPA].m(C)[sP].f(A)[sP].f(G)[msPA].f(A)[sP].f(A)[sP].f(G) |
| | [sP].m(C)[sP].f(A)[sP].f(A)[sP].m(U)[sP].f(G)[sP].d(C)[sP].[Dh](C)[sP].d(I) |
| | [msPA].m(U)[P].f(C)[sP].m(A)[sP].m(C)[msPA].m(C)} (SEQ ID NO: 7) |
| A8 | {m(C)[msPA].f(A)[sP].m(U)[sP].f(A)[sP].f(A)[sP].m(U)[sP].m(U)[sP].m(U)[sP].f |
| | (A)[sP].m(C)[sP].f(A)[msPA].m(C)[sP].f(A)[sP].f(G)[msPA].f(A)[P].f(A)[sP].f |
| | (G)[sP].m(C)[sP].f(A)[sP].f(A)[sP].m(U)[sP].f(G)[sP].d(C)[P].[Dh](C)[sP].d(I) |
| | [msPA].m(U)[sP].f(C)[sP].m(A)[sP].m(C)[msPA].m(C) (SEQ ID NO: 8) |
| A9 | m(C)[msPA].f(A)[sP].m(U)[sP].f(A)[sP].f(A)[sP].m(U)[sP].m(U)[sP].m(U)[sP].f |
| | (A)[sP].m(C)[sP].f(A)[msPA].m(C)[sP].f(A)[sP].f(G)[msPA].f(A)[sP].f(A)[sP].f |
| | (G)[sP].m(C)[sP].f(A)[P].f(A)[sP].m(U)[sP].f(G)[sP].d(C)[sP].[Dh](C)[sP].d(I) |
| | [msPA].m(U)[sP].f(C)[sP].m(A)[sP].m(C)[msPA].m(C)P.d(A)P.d(A)}\| (SEQ ID NO: 9) |
| A10 | {d(A)P.d(A)P.m(C)[msPA].f(A)[sP].m(U)[sP].f(A)[P].f(A)[sP].m(U)[sP].m(U)[sP]. |
| | m(U)[sP].f(A)[sP].m(C)[P].f(A)[msPA].m(C)[sP].f(A)[sP].f(G)[msPA].f(A)[sP].f |
| | (A)[sP].f(G)[sP].m(C)[sP].f(A)[sP].f(A)[sP].m(U)[sP].f(G)[sP].d(C)[sP].[Dh] |
| | (C)[sP].d(I)[msPA].m(U)[sP].f(C)[sP].m(A)[sP].m(C)[msPA].m(C)} (SEQ ID NO: 10) |
| A11 | {m(C)[msPA].m(C)[sP].m(C)[sP].f(A)[sP].f(G)[sP].[moe]([m5C])P.[moe](A)P.f(G) |
| | [sP].f(C)[sP].[moe](T)P.f(U)[msPA].m(C)P.f(A)[sP].m(G)[msPA].f(U)P.m(C)[sP].f |
| | (C)[sP].f(C)[sP].f(U)[sP].[moe](T)P.[moe](T)P.f(C)[sP].d(T)[sP].[Dh](C)[sP].d |
| | (I)[msPA].m(U)P.f(C)[sP].m(G)[sP].m(A)[msPA].m(U) (SEQ ID NO: 11) |
| A12 | {[moe](A)[msPA].[moe](A)[sP].m(C)P.m(A)[sP].f(U)[P].f(G)[sP].f(G)[sP].f(C) |
| | [sP].m(C)[sP].f(C)[sP].m(C)[sP].f(A)[sP].m(G)P.m(C)[sP].f(A)[sP].f(G)[sP].m |
| | (C)[sP].f(U)[sP].m(U)P.m(C)P.m(A)[msPA].f(G)[sP].f(U)[msPA].m(C)[sP].f(C)[sP]. |
| | f(C)[sP].f(U)[sP].m(U)[sP].f(U)[sP].m(C)[sP].d(T)[sP].[Dh](C)[sP].[fana](I) |
| | [msPA].m(U)[sP].f(C)[sP].m(G)P.m(A)P.m(U)P.m(G)[sP].f(G)[sP].[moe](T)[msPA]. |
| | [moe]([m5C]) (SEQ ID NO: 12) |
| A13 | {[moe](A)[msPA].[moe](A)[sP].m(C)P.m(A)[sP].f(U)[sP].f(G)[P].f(G)[sP].f(C) |
| | [sP].m(C)[sP].f(C)[sP].m(C)[sP].f(A)[sP].m(G)P.m(C)[P].f(A)[sP].f(G)[sP].m(C) |
| | [sP].f(U)[sP].m(U)P.m(C)P.m(A)[msPA].f(G)[sP].f(U)[msPA].m(C)[sP].f(C)[sP].f |
| | (C)[sP].f(U)[sP].m(U)[sP].f(U)[sP].m(C)[sP].d(T)[sP].[Dh](C)[sP].[fana](I) |
| | [msPA].m(U)[sP].f(C)[sP].m(G)P.m(A)P.m(U)P.m(G)[sP].f(G)[sP].[moe](T)[msPA]. |
| | [moe]([m5C])} (SEQ ID NO: 13) |
| A14 | {m(C)[msPA].f(C)[sP].f(C)[sP].f(C)[sP].m(A)[sP].f(G)[sP].f(C)[sP].f(A)[sP].m |
| | (G)[sP].f(C)[sP].f(U)[sP].f(U)[sP].m(C)[sP].f(A)[msPA].f(G)[sP].f(U)[msPA].m |
| | (C)[sP].f(C)[sP].f(C)[sP].f(U)[sP].m(U)[sP].f(U)[sP].f(C)[sP].f(U)[sP].[Dh] |
| | (C)[sP].d(I)[msPA].m(U)[sP].f(C)[sP].m(G)[msPA].m(A)} (SEQ ID NO: 14) |
| A15 | {m(C)[msPA].f(C)[sP].f(C)[sP].f(C)[sP].m(A)[sP].f(G)[sP].f(C)[sP].f(A)[sP].m |
| | (G)[sP].f(C)[sPJ.f(U)[sP].f(U)[sP].m(C)[sP].f(A)[msPA].f(G)[sP].f(U)[msPA].m |
| | (C)[sP].f(C)[sP].f(C)[sP].f(U)[sP].m(U)[sP].f(U)[sP].f(C)[sP].f(U)[sP].[Dh] |
| | (C)[sP].d(I)[msPA].m(U)[sP].f(C)[sP].m(G)[msPA].m(A)} (SEQ ID NO: 15) |
| A16 | {m(C)[sP].m(A)[sP].m(U)[sP].m(A)[sP].f(A)[sP].m(U)P.m(U)[sP].m(C)P.m(U)[sP].m |
| | (U)P.f(G)[sP].m(U)P.f(G)[sP].m(U)P.f(C)[sP].m(U)P.f(A)[sP].f(C)[sP].m(U)P.f(G) |
| | [sP].m(U)P.f(A)[sP].f(C)[sP].f(A)[sP].f(G)[sP].f(A)[sP].f(A)[sP].m(U)P.f(A) |
| | [sP].f(C)[sP].f(U)[sP].m(G)[sP].m(C)P.f(C)[sP].m(G)P.d(C)[sP].d(C)[sP].d(A) |
| | [sP].m(G)P.m(C)P.m(U)P.m(G)P.m(G)P.m(A)P.m(U)P.m(U)[sP].m(U)[sP].m(C)[sP].m |
| | (C)[sP].m(C)} (SEQ ID NO: 16) |
| A17 | {m(C)[sP].m(A)[sP].m(U)[sP].m(A)[sP].f(A)[sP].m(U)P.m(U)[sP].m(C)P.m(U)[sP].m |
| | (U)P.f(G)[sP].m(U)P.f(G)[sP].m(U)P.f(C)[sP].m(U)P.f(A)[sP].f(C)[sP].m(U)P.f |
| | (G)[sP].m(U)P.f(A)[sP].f(C)[sP].f(A)[sP].f(G)[sP].f(A)[sP].f(A)[sP].m(U)P.f |
| | (A)[sP].f(C)[sP].f(U)[sP].m(G)[sP].m(C)P.f(C)[sP].m(G)P.d(C)[sP].d(C)[sP].d |
| | (A)[sP].m(G)P.m(C)P.m(U)P.m(G)P.m(G)P.m(A)P.m(U)P.m(U)[sP].m(U)[sP].m(C)[sP]. |
| | m(C)[sP].m(C) (SEQ ID NO: 17) |
| A18 | {m(C)[sP].m(A)[sP].m(U)[sP].m(A)[sP].f(A)[sP].m(U)P.m(U)[sP].m(C)P.m(U)[sP].m |
| | (U)P.f(G)[sP].m(U)P.f(G)[sP].m(U)P.f(C)[sP].m(U)P.f(A)[sP].f(C)[sP].m(U)P.f(G) |
| | [sP].m(U)P.f(A)[sP].f(C)[sP].f(A)[sP].f(G)[sP].f(A)[sP].f(A)[sP].m(U)P.f(A) |
| | [sP].f(C)[sP].f(U)[sP].m(G)[sP].m(C)P.f(C)[sP].m(G)P.d(C)[sP].d(C)[sP].d(A) |
| | [sP].m(G)P.m(C)P.m(U)P.m(G)P.m(G)P.m(A)P.m(U)P.m(U)[sP].m(U)[sP].m(C)[sP].m |
| | (C)[sP].m(C)} (SEQ ID NO: 18) |
| A19 | {m(G)[sP].m(G)[sP].m(U)P.f(G)[sP].m(U)P.m(C)P.f(G)[sP].f(A)[sP].f(G)[sP].f(A) |
| | [sP].f(A)[sP].f(G)[sP].f(A)[sP].f(G)[sP].f(G)[sP].f(A)[sP].f(G)[sP].f(A)[sP]. |
| | f(A)[sP].m(C)P.f(A)[sP].f(A)[sP].m(U)P.f(A)[sP].m(U)P.f(G)[sP].m(C)P.m(U)P.f |
| | (A)[sP].f(A)[sP].f(A)[sP].m(U)P.f(G)[sP].m(U)P.m(U)P.f(G)[sP].m(U)P.m(U)P.m |
| | (C)P.m(U)P.m(C)P.f(G)[sP].m(U)P.m(C)P.m(U)P.m(C)P.m(C)P.m(U)P.m(C)P.f(G)[sP]. |
| | f(A)[sP].m(C)P.f(A)[sP].m(C)P.m(C)P.m(C)P.m(U)P.m(G)P.m(C)P.m(C)P.m(G)P.d(C) |
| | [sP].d(C)[sP].d(A)[sP].m(G)P.m(C)P.m(U)P.m(G)P.m(G)[sP].m(A)[sP].m(U)[sP].m |
| | (U)[sP].m(U)} (SEQ ID NO: 19) |
| A20 | {m(G)[sP].m(G)[sP].m(U)P.f(G)[sP].m(U)P.m(C)P.f(G)[sP].f(A)[sP].f(G)[sP].f(A) |
| | [sP].f(A)[sP].f(G)[sP].f(A)[sP].f(G)[sP].f(G)[sP].f(A)[sP].f(G)[sP].f(A)[sP]. |
| | f(A)[sP].m(C)P.f(A)[sP].f(A)[sP].m(U)P.f(A)[sP].m(U)P.f(G)[sP].m(C)P.m(U)P.f |
| | (A)[sP].f(A)[sP].f(A)[sP].m(U)P.f(G)[sP].m(U)P.m(U)P.f(G)[sP].m(U)P.m(U)P.m |
| | (C)P.m(U)P.m(C)P.f(G)[sP].m(U)P.m(C)P.m(U)P.m(C)P.m(C)P.m(U)P.m(C)P.f(G)[P].f |
| | (A)[sP].m(C)P.f(A)[sP].m(C)P.m(C)P.m(C)P.m(U)P.m(G)P.m(C)P.m(C)P.m(G)P.d(C) |
| | [sP].d(C)[sP].d(A)[sP].m(G)P.m(C)P.m(U)P.m(G)P.m(G)[sP].m(A)[sP].m(U)[sP].m |
| | (U)[sP].m(U) (SEQ ID NO: 20) |
| A21 | {m(C)[sP].m(A)[sP].m(U)[sP].m(A)[sP].f(A)[sP].m(U)P.m(U)[sP].m(C)P.m(U)[sP].m |
| | (U)P.f(G)[sP].m(U)P.f(G)[sP].m(U)P.f(C)[sP].m(U)P.f(A)[sP].f(C)[sP].m(U)P.f |
| | (G)[sP].m(U)P.f(A)[sP].f(C)[sP].f(A)[sP].f(G)[sP].f(A)[sP].f(A)[sP].m(U)P.f |
| | (A)[sP].f(C)[sP].f(U)[sP].m(G)[sP].m(C)P.f(C)[sP].m(G)P.d(C)[sP].d(C)[sP].d |
| | (A)[sP].m(G)P.m(C)P.m(U)P.m(G)P.m(G)P.m(A)P.m(U)P.m(U)[sP].m(U)[sP].m(C)[sP]. |
| | m(C)[sP].m(C)} (SEQ ID NO: 21) |
| A22 | {m(C)[sP].m(A)[sP].m(U)[sP].m(A)[sP].f(A)[sP].m(U)P.m(U)[sP].m(C)P.m(U)[sP].m |
| | (U)P.f(G)[sP].m(U)P.f(G)[sP].m(U)P.f(C)[sP].m(U)P.f(A)[sP].f(C)[sP].m(U)P.f |
| | (G)[sP].m(U)P.f(A)[sP].f(C)[sP].f(A)[sP].f(G)[sP].f(A)[sP].f(A)[sP].m(U)P.f |
| | (A)[sP].f(C)[sP].f(U)[sP].m(G)[sP].m(C)P.f(C)[sP].m(G)P.d(C)[sP].d(C)[sP].d |
| | (A)[sP].m(G)P.m(C)P.m(U)P.m(G)P.m(G)P.m(A)P.m(U)P.m(U)[sP].m(U)[sP].m(C)[sP]. |
| | m(C)[sP].m(C)} (SEQ ID NO: 22) |
| A23 | {m(C)[sP].m(A)[sP].m(U)[sP].m(A)[P].f(A)[sP].m(U)P.m(U)[sP].m(C)P.m(U)[sP].m |
| | (U)P.f(G)[sP].m(U)P.f(G)[sP].m(U)P.f(C)[sP].m(U)P.f(A)[sP].f(C)[sP].m(U)P.f |
| | (G)[sP].m(U)P.f(A)[sP].f(C)[sP].f(A)[sP].f(G)[sP].f(A)[sP].f(A)[sP].m(U)P.f |
| | (A)[sP].f(C)[sP].f(U)[sP].m(G)[sP].m(C)P.f(C)[sP].m(G)P.d(C)[sP].d(C)[sP].d |
| | (A)[sP].m(G)P.m(C)P.m(U)P.m(G)P.m(G)P.m(A)P.m(U)P.m(U)[sP].m(U)[sP].m(C)[sP]. |
| | m(C)[sP].m(C)} (SEQ ID NO: 23) |
| A24 | {m(C)[sP].m(A)[sP].m(U)[sP].m(A)[sP].f(A)[sP].m(U)P.m(U)[sP].m(C)P.m(U)[sP].m |
| | (U)P.f(G)[sP].m(U)P.f(G)[sP].m(U)P.f(C)[sP].m(U)P.f(A)[sP].f(C)[sP].m(U)P.f |
| | (G)[sP].m(U)P.f(A)[sP].f(C)[sP].f(A)[sP].f(G)[sP].f(A)[sP].f(A)[sP].m(U)P.f |
| | (A)[sP].f(C)[sP].f(U)[sP].m(G)[sP].m(C)P.f(C)[sP].m(G)P.d(C)[sP].d(C)[sP].d |
| | (A)[sP].m(G)P.m(C)P.m(U)P.m(G)P.m(G)P.m(A)P.m(U)P.m(U)[sP].m(U)[sP].m(C)[sP]. |
| | m(C)[sP].m(C)P.d(A)P.d(A)} (SEQ ID NO: 24) |
| A25 | {d(A)P.d(A)P.m(C)[sP].m(A)[sP].m(U)[sP].m(A)[sP].f(A)[sP].m(U)P.m(U)[sP].m(C) |
| | P.m(U)[sP].m(U)P.f(G)[sP].m(U)P.f(G)[sP].m(U)P.f(C)[sP].m(U)P.f(A)[P].f(C) |
| | [sP].m(U)P.f(G)[sP].m(U)P.f(A)[sP].f(C)[sP].f(A)[sP].f(G)[sP].f(A)[sP].f(A) |
| | [sP].m(U)P.f(A)[sP].f(C)[sP].f(U)[sP].m(G)[sP].m(C)P.f(C)[sP].m(G)P.d(C)[sP]. |
| | d(C)[sP].d(A)[sP].m(G)P.m(C)P.m(U)P.m(G)P.m(G)P.m(A)P.m(U)P.m(U)[sP].m(U)[sP]. |
| | m(C)[sP].m(C)[sP].m(C)} (SEQ ID NO: 25) |
| A26 | {m(G)[sP].m(U)[sP].m(G)[sP].m(U)[sP].f(C)[sP].m(U)[sP].m(A)P.f(C)[sP].m(U)P.m |
| | (G)[sP].m(U)P.f(A)[sP].f(C)[sP].m(A)[sP].m(G)P.m(C)[sP].m(A)P.f(U)[sP].m(A) |
| | P.f(C)[sP].f(U)[sP].m(G)[sP].m(C)P.f(C)[sP].m(G)P.d(C)[sP].d(C)[sP].d(A)[sP]. |
| | m(G)P.f(C)[sP].m(U)P.m(G)[sP].f(G)[sP].f(A)[sP].m(U)P.f(U)[sP].m(U)P.m(G)[sP]. |
| | m(C)P.m(C)[sP].f(A)[sP].f(A)[sP].m(U)P.m(U)[sP].m(C)P.f(U)[sP].m(G)P.m(A)[sP]. |
| | f(G)[sP].m(U)[sP].m(A)[sP].m(A)[sP].m(C)} (SEQ ID NO: 26) |
| A27 | {m(G)[sP].m(U)[sP].m(G)[sP].m(U)[sP].f(C)[sP].m(U)[sP].m(A)P.f(C)[sP].m(U)P.m |
| | (G)[sP].m(U)P.f(A)[sP].f(C)[sP].m(A)[sP].m(G)P.m(C)[sP].m(A)P.f(U)[sP].m(A)P. |
| | f(C)[sP].f(U)[sP].m(G)[sP].m(C)P.f(C)[sP].m(G)P.d(C)[sP].d(C)[sP].d(A)[P].m |
| | (G)P.f(C)[sP].m(U)P.m(G)[sP].f(G)[sP].f(A)[sP].m(U)P.f(U)[sP].m(U)P.m(G)[sP]. |
| | m(C)P.m(C)[sP].f(A)[sP].f(A)[sP].m(U)P.m(U)[sP].m(C)P.f(U)[sP].m(G)P.m(A)[sP]. |
| | f(G)[sP].m(U)[sP].m(A)[sP].m(A)[sP].m(C)} (SEQ ID NO: 27) |
| A28 | {m(G)[sP].m(U)[sP].m(G)[sP].m(U)[sP].f(C)[sP].m(U)[sP].m(A)P.f(C)[sP].m(U)P.m |
| | (G)[sP].m(U)P.f(A)[sP].f(C)[sP].m(A)[sP].m(G)P.m(C)[P].m(A)P.f(U)[sP].m(A)P.f |
| | (C)[sP].f(U)[sP].m(G)[sP].m(C)P.f(C)[sP].m(G)P.d(C)[sP].d(C)[sP].d(A)[sP].m |
| | (G)P.f(C)[sP].m(U)P.m(G)[sP].f(G)[sP].f(A)[sP].m(U)P.f(U)[sP].m(U)P.m(G)[sP]. |
| | m(C)P.m(C)[sP].f(A)[sP].f(A)[sP].m(U)P.m(U)[sP].m(C)P.f(U)[sP].m(G)P.m(A)[sP]. |
| | f(G)[sP].m(U)[sP].m(A)[sP].m(A)[sP].m(C)} (SEQ ID NO: 28) |
| A29 | {m(G)[sP].m(U)[sP].m(G)[sP].m(U)[sP].f(C)[sP].m(U)[sP].m(A)P.f(C)[sP].m(U)P.m |
| | (G)[sP].m(U)P.f(A)[sP].f(C)[sP].m(A)[sP].m(G)P.m(C)[sP].m(A)P.f(U)[sP].m(A)P. |
| | f(C)[sP].f(U)[sP].m(G)[sP].m(C)P.f(C)[sP].m(G)P.d(C)[sP].d(C)[sP].d(A)[sP].m |
| | (G)P.f(C)[sP].m(U)P.m(G)[sP].f(G)[sP].f(A)[sP].m(U)P.f(U)[sP].m(U)P.m(G)[sP]. |
| | m(C)P.m(C)[sP].f(A)[sP].f(A)[sP].m(U)P.m(U)[sP].m(C)P.f(U)[sP].m(G)P.m(A)[sP]. |
| | f(G)[sP].m(U)[sP].m(A)[sP].m(A)[sP].m(C)} (SEQ ID NO: 29) |
| A30 | {m(G)[sP].m(U)[sP].m(G)[sP].m(U)[sP].f(C)[sP].m(U)[sP].m(A)P.f(C)[sP].m(U)P.m |
| | (G)[sP].m(U)P.f(A)[sP].f(C)[sP].m(A)[sP].m(G)P.m(C)[sP].m(A)P.f(U)[sP].m(A) |
| | P.f(C)[sP].f(U)[sP].m(G)[sP].m(C)P.f(C)[sP].m(G)P.d(C)[P].d(C)[sP].d(A)[sP]. |
| | m(G)P.f(C)[sP].m(U)P.m(G)[sP].f(G)[sP].f(A)[sP].m(U)P.f(U)[sP].m(U)P.m(G) |
| | [sP].m(C)P.m(C)[sP].f(A)[sP].f(A)[sP].m(U)P.m(U)[sP].m(C)P.f(U)[sP].m(G)P.m |
| | (A)[sP].f(G)[sP].m(U)[sP].m(A)[sP].m(A)[sP].m(C)} (SEQ ID NO: 30) |
| A31 | {m(G)[sP].m(U)[sP].m(G)[sP].m(U)[sP].f(C)[sP].m(U)[sP].m(A)P.f(C)[sP].m(U)P.m |
| | (G)[sP].m(U)P.f(A)[sP].f(C)[sP].m(A)[sP].m(G)P.m(C)[sP].m(A)P.f(U)[sP].m(A) |
| | P.f(C)[sP].f(U)[sP].m(G)[sP].m(C)P.f(C)[sP].m(G)P.d(C)[sP].d(C)[sP].d(A)[sP]. |
| | m(G)P.f(C)[sP].m(U)P.m(G)[sP].f(G)[sP].f(A)[sP].m(U)P.f(U)[sP].m(U)P.m(G)[sP]. |
| | m(C)P.m(C)[sP].f(A)[sP].f(A)[sP].m(U)P.m(U)[sP].m(C)P.f(U)[sP].m(G)P.m(A)[P]. |
| | f(G)[sP].m(U)[sP].m(A)[sP].m(A)[sP].m(C) (SEQ ID NO: 31) |
| A32 | {[moe]([m5C])[msPA].[moe]([m5C])[sP].[moe]([m5C])[msPA].f(A)[sP].f(G)[sP].m |
| | (C)P.m(A)P.f(G)[msPA].f(C)[sP].m(U)P.m(U)P.m(C)P.m(A)P.m(G)P.f(U)[msPA].[moe] |
| | ([m5C])P.f(C)[sP].[moe]([m5C])P.f(U)[sP].m(U)P.[moe](T)P.f(C)[sP].d(T)[sP]. |
| | [Dh](C)[sP].d(I)[msPA].m(U)P.f(C)[sP].m(G)[sP].f(A)[msPA].m(U)} (SEQ ID NO: |
| | 32) |
| A33 | {m(C)[msPA].[moe]([m5C])[sP].[moe]([m5C])[msPA].f(A)[sP].f(G)[sP].m(C)P.m(A) |
| | P.f(G)[mSPAJ.f(C)[sP].[moe](T)P.[moe](T)P.m(C)P.m(A)P.m(G)P.f(U)[msPA].[moe] |
| | ([m5C])P.f(C)[sP].m(C)P.f(U)[sP].m(U)P.[moe](T)P.f(C)[sP].d(T)[sP].[Dh](C) |
| | [sP].d(I)[msPA].m(U)P.f(C)[sP].m(G)[sP].f(A)[msPA].m(U)} (SEQ ID NO: 33) |
| A34 | {[moe]([m5C])[msPA].[moe]([m5C])[sP].[moe]([m5C])[msPA].f(A)[sP].f(G)[sP].m |
| | (C)P.m(A)P.f(G)[msPA].f(C)[sP].m(U)P.m(U)P.m(C)P.m(A)P.m(G)P.f(U)[msPA].[moe] |
| | ([m5C])P.f(C)[sP].[moe]([m5C])P.f(U)[sP].m(U)P.[moe](T)P.f(C)[sP].d(T)[sP]. |
| | [Dh](C)[sP].d(I)[msPA].m(U)P.f(C)[sP].m(G)[sP].f(A)[msPA].m(U)} (SEQ ID NO: |
| | 34) |

TABLE 6
Nucleotide Sequences

| Oligo | | SEQ |
|---|---|---|
| No. | Base sequences | ID NO: |
| A1 | CCCAGAGCTUCAGUCCCUTTCTCIUCGAU | 35 |
| A2 | CCCAGAGCTUCAGUCCCUTTCTCIUCGAU | 36 |
| A3 | CCCAGAGCTUCAGUCCCUTTCTCIUCGAU | 37 |
| A4 | CCCAGAGCTUCAGUCCCUTTCTCIUCGAU | 38 |
| A5 | CAUAAUUUACACAGAAGCAAUGCCIUCACC | 39 |
| A6 | CAUAAUUUACACAGAAGCAAUGCCIUCACC | 40 |
| A7 | CAUAAUUUACACAGAAGCAAUGCCIUCACC | 41 |
| A8 | CAUAAUUUACACAGAAGCAAUGCCIUCACC | 42 |
| A9 | CAUAAUUUACACAGAAGCAAUGCCIUCACCAA\| | 43 |
| A10 | AACAUAAUUUACACAGAAGCAAUGCCIUCACC | 44 |
| A11 | CCCAGAGCTUCAGUCCCUTTCTCIUCGAU | 45 |
| A12 | AACAUGGCCCCAGCAGCUUCAGUCCCUUUCTCIUCGAUGGT | 46 |
| A13 | AACAUGGCCCCAGCAGCUUCAGUCCCUUUCTCIUCGAUGGT | 47 |
| A14 | CCCCAGCAGCUUCAGUCCCUUUCUCIUCGA | 48 |
| A15 | CCCCAGCAGCUUCAGUCCCUUUCUCIUCGA | 49 |
| A16 | CAUAAUUCUUGUGUCUACUGUACAGAAUACUGCCGCCAGCUGGAUUUCCC | 50 |
| A17 | CAUAAUUCUUGUGUCUACUGUACAGAAUACUGCCGCCAGCUGGAUUUCCC | 51 |
| A18 | CAUAAUUCUUGUGUCUACUGUACAGAAUACUGCCGCCAGCUGGAUUUCCC | 52 |
| A19 | GGUGUCGAGAAGAGGAGAACAAUAUGCUAAAUGUUGUUCUCGUCUCCUCGACA | 53 |
| | CCCUGCCGCCAGCUGGAUUU | |
| A20 | GGUGUCGAGAAGAGGAGAACAAUAUGCUAAAUGUUGUUCUCGUCUCCUCGACA | 54 |
| | CCCUGCCGCCAGCUGGAUUU | |
| A21 | CAUAAUUCUUGUGUCUACUGUACAGAAUACUGCCGCCAGCUGGAUUUCCC | 55 |
| A22 | CAUAAUUCUUGUGUCUACUGUACAGAAUACUGCCGCCAGCUGGAUUUCCC | 56 |
| A23 | CAUAAUUCUUGUGUCUACUGUACAGAAUACUGCCGCCAGCUGGAUUUCCC | 57 |
| A24 | CAUAAUUCUUGUGUCUACUGUACAGAAUACUGCCGCCAGCUGGAUUUCCCAA | 58 |
| A25 | AACAUAAUUCUUGUGUCUACUGUACAGAAUACUGCCGCCAGCUGGAUUUCCC | 59 |
| A26 | GUGUCUACUGUACAGCAUACUGCCGCCAGCUGGAUUUGCCAAUUCUGAGUAAC | 60 |
| A27 | GUGUCUACUGUACAGCAUACUGCCGCCAGCUGGAUUUGCCAAUUCUGAGUAAC | 61 |
| A28 | GUGUCUACUGUACAGCAUACUGCCGCCAGCUGGAUUUGCCAAUUCUGAGUAAC | 62 |
| A29 | GUGUCUACUGUACAGCAUACUGCCGCCAGCUGGAUUUGCCAAUUCUGAGUAAC | 63 |
| A30 | GUGUCUACUGUACAGCAUACUGCCGCCAGCUGGAUUUGCCAAUUCUGAGUAAC | 64 |
| A31 | GUGUCUACUGUACAGCAUACUGCCGCCAGCUGGAUUUGCCAAUUCUGAGUAAC | 65 |
| A32 | AGCAGCUUCAGUCUUTCTCIUCGAU | 66 |
| A33 | CAGCAGCTTCAGUCCUUTCTCIUCGAU | 67 |
| A34 | AGCAGCUUCAGUCUUTCTCIUCGAU | 68 |

### Example 2—Editing Efficiency of GalNAc Oligonucleotides on Mouse ACTB

The uptake and editing efficiency of GalNAc oligonucleotides disclosed herein on mouse actin beta (ACTB) were studied in vitro and in vivo.

### Comparison of 3′ and 5′ Prolinol (GP) and 5′ Extended (GE) on 30-Mers (In Vitro Editing)

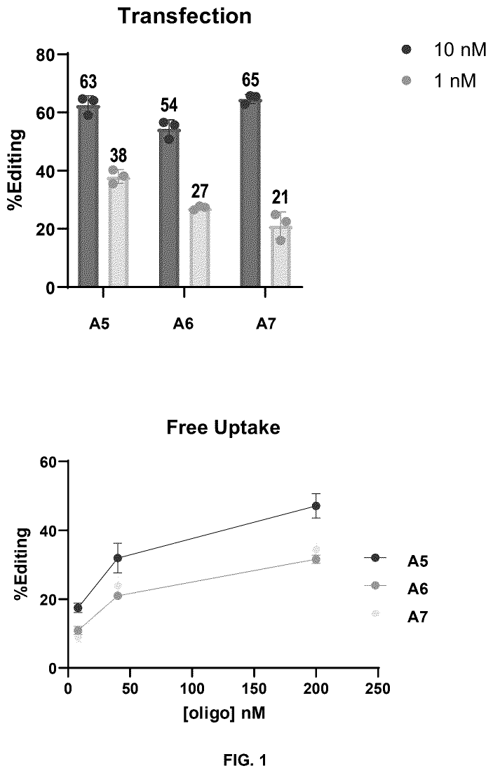

A study of in vitro editing of ACTB by GalNAc oligonucleotides described herein through transfection or free uptake was undertaken. Editing was measured in primary mouse hepatocytes. Mouse hepatocytes were thawed in a 37° C. water bath in a 50 ml tube of Lonza rodent cryopreserved hepatocyte thawing media (MCRT50-Lonza). After centrifugation at 100×g for 5 minutes, supernatant was aspirated and the cell pellet is resuspended in Lonza plating media (Lonza). Cells were plated on 96-well collagen-coated tissue culture plates at 20000 cells/well. Cells were transferred to incubator (37° C.), 4 to 6 hours later media was changed to Lonza maintenance media (CC-3198, LonzA) and cells were transfected with ASOs at desired concentrations, with and without RNAiMax (Life Technologies, CA) according to manufacturer's protocol and transferred to a 37° C. incubator. Primers for NGS are shown below in Table 7. Data are shown in FIG. 1.

TABLE 7
PCR and sequencing primers

| | | |
|---|---|---|
| SEQ ID NO: 69 | Forward primer | TAAGTGGTTACAGGAAGT |
| | Mouse ACTB Site 1 | CCCTCA |
| SEQ ID NO: 70 | Reverse primer | GGAGGCCTCAGACCTGGG |
| | Mouse ACTB Site 1 | CCA |

30-mer oligonucleotides containing a 5′ GP, 3′ GP, or 5′ GE modification effectively edited ACTB in vitro, with 5′ GP and 5′ GE showing approximately equal efficiency, which was slightly lower than 3′ GP efficiency.

### Comparison of 3′ and 5′ Prolinol (GP) and 5′ Extended (GE) on 30-Mers (In Vivo Editing)

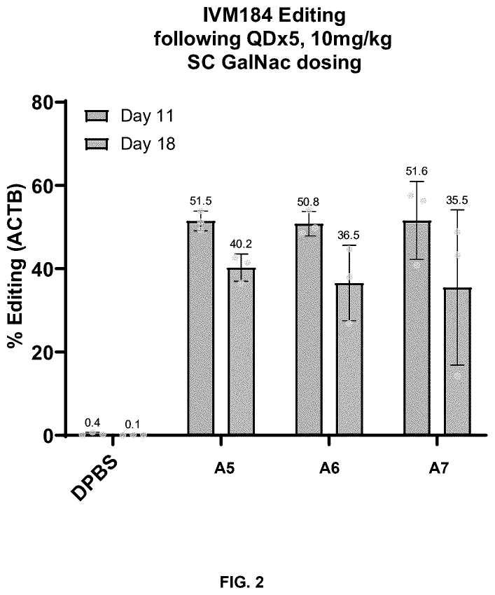

A study of in vivo editing of ACTB by GalNAc oligonucleotides described herein in wild-type (WT) mice was undertaken. Mice were injected subcutaneously with oligonucleotides daily for five consecutive days with each dose at 10 mg/kg (QDx5 at 10 mg/kg). Livers were isolated from mice eleven and eighteen days after the first injection, and total RNA was isolated. The isolated RNA was used for cDNA synthesis using SuperScript IV VILO™ according to the manufacturer's instructions (Life Technologies). Ten μl of the cDNA was used for Next Generation Sequencing (NGS), Amplicon Sequencing by Quintara Biosciences. Percent editing of the site of interest was quantified as a percentage of the number of edited nucleotides based on NGS counts. Each oligonucleotide was assayed in at least three replicates. Primer sequences used for NGS are shown below in Table 8. Data are shown in FIG. 2.

TABLE 8
PCR and sequencing primers

| | |
|---|---|
| Forward primer | TAAGTGGTTACAGGAAGTCCCTCA |
| Mouse ACTB Site 1 | (SEQ ID NO: 71) |
| Reverse primer | GGAGGCCTCAGACCTGGGCCA |
| Mouse ACTB Site 1 | (SEQ ID NO: 72) |

30-mer oligonucleotides containing a 5′ GP, 3′ GP, or 5′ GE modification effectively edited ACTB in vivo as measured at days 11 and 18.

Despite some differences in transfection and free uptake activity, all GalNAc linker designs showed similar editing activity at days 7 and 14 post-last dose in vivo. Some variability in target editing was seen with 5′ GE, especially at day 14 post-dose.

### Evaluation of 3′ GP Oligonucleotides Delivered Subcutaneously in Liver

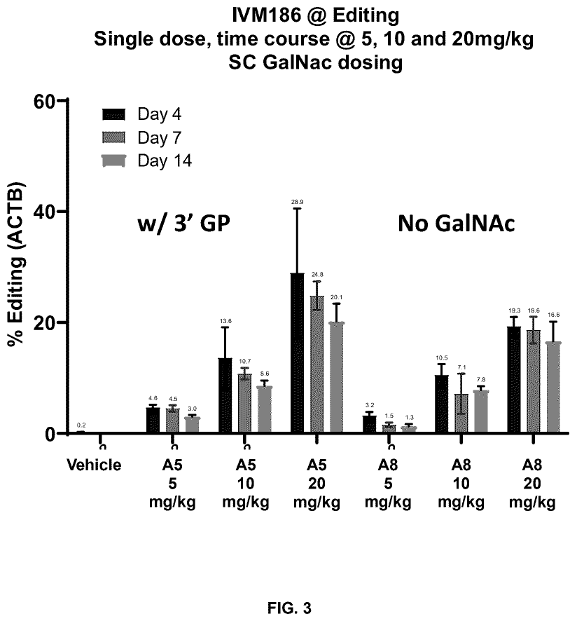

A study of in vivo editing of ACTB by GalNAc oligonucleotides described herein in the livers of wild-type (WT) mice was undertaken. WT mice (n=3) were treated with 30-mer oligonucleotides disclosed herein (either 3′ GP modified, or unmodified) administered subcutaneously at 5, 10, or 20 mg/kg (single dose). Takedowns were performed on days 4, 7, and 14 post last dose. Data are shown in FIG. 3.

3′ GalNAc appears to boost editing seven days after a single injection at three different dose levels compared to unmodified oligonucleotides.

### Example 3—Editing Efficiency of GalNAc Oligonucleotides on E342K

The uptake and editing efficiency of GalNAc oligonucleotides disclosed herein on the E342K mutation in PiZ mice were studied in vitro and in vivo. PiZ mice express mutant human SERPINA1 (also known as AAT or α1-antitrypsin), which carries a glutamic acid to lysine substitution at residue 342 (E342K).

5′ Decanol GalNAc (5′ GD) Boosts Editing In Vivo Vs. No GalNAc

A study of in vitro and in vivo editing of E342K by GalNAc oligonucleotides described herein through transfection or free uptake was undertaken. Editing efficiency of oligos was tested in Piz Mouse Hepatocytes without interferon alpha. These were tested using RNAiMAX (“transfection”), as well as without any lipofectamine (“free uptake”) at the desired concentrations.

Piz Mouse Hepatocytes were thawed in a 37° C. water bath and mixed with 50 ml of Cryopreserved Hepatocyte Recovery Medium (CHRM—Life Technologies). After centrifugation at 80×g for 6 minutes, supernatant was aspirated and the cell pellet is resuspended in Hepatocyte Plating Media (MB Bioscience). Cells were plated on to either 96-well collagen-coated tissue culture plates at 20000 cells/well. Cells were transferred to incubator (37° C.), 4 to 6 hours later media was changed to Hepatocyte Maintenance Media (MB Bioscience) and cells were transfected with ASOs at desired concentrations, with and without RNAiMax (Life Technologies, CA) according to manufacturer's protocol and placed back into the incubator.

48 hours after the addition of oligo, mRNA was isolated from the PiZ hepatocytes using Oligo(dT)25 magnetic beads and relevant buffers from New England BioLabs. The samples were treated with EZ DNase (Life Technologies) after elution. The resultant isolated mRNA was used for cDNA synthesis using SuperScript IV VILO™ according to the manufacturer's instructions (Life Technologies). Ten μl of the cDNA was used for Next Generation Sequencing (NGS), Amplicon Sequencing by Quintara Biosciences.

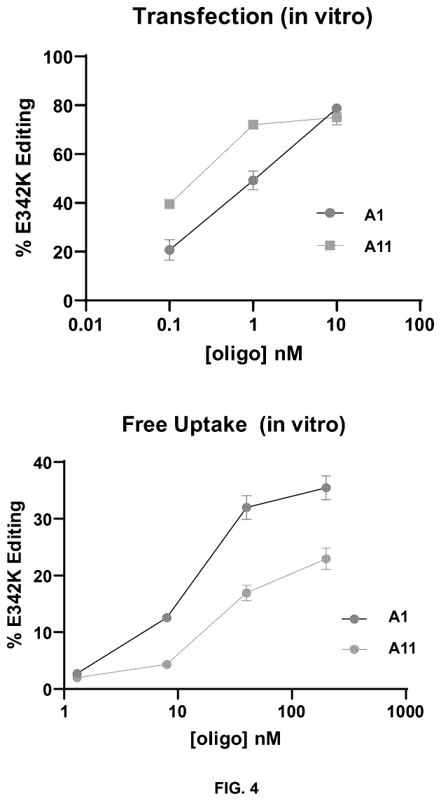

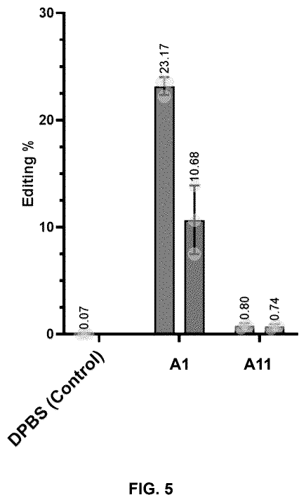

The DNA amplicons were directly used for Amplicon Next Generation Sequencing (NGS). Percent editing of the site of interest was quantified as a percentage of the number of edited nucleotides based on NGS counts. Each oligonucleotide was assayed in at least three replicates. Primers used for NGS are shown in Table 9. Data are shown in FIGS. 4 (in vitro) and 5 (in vivo).

TABLE 9
PCR and sequencing primers

| | | |
|---|---|---|
| SEQ ID NO: 73 | Forward primer | ACCTATGATCTGAAGAGCG |
| | E342K Site E342K | TCCT |
| SEQ ID NO: 74 | Reverse primer | TTCAATCATTAAGAAGACA |
| | E342K Site E342K | AAGGGT |

Removal of 5′ GD resulted in decreased activity by free uptake and complete loss of activity (A11) in vivo.

### Comparison of 5′ Extended (GE) and 3′ Prolinol (GP) on a 42Mer and 30Mer (In Vitro)

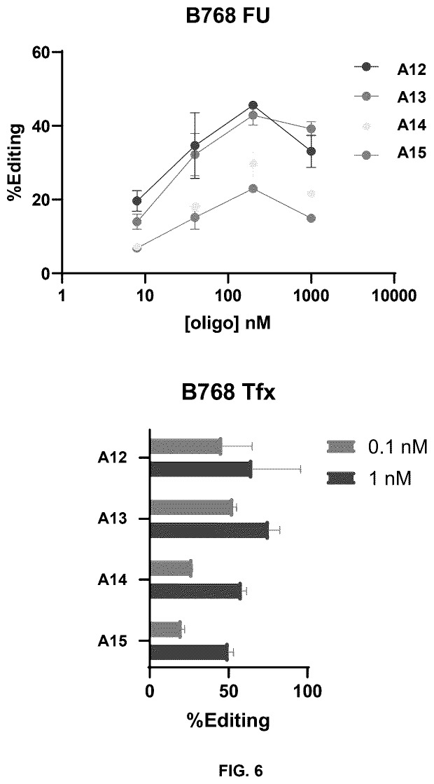

A study of in vitro editing of E342K by GalNAc oligonucleotides described herein as measured by FU and Tfx in PiZ mouse hepatocytes was undertaken. Editing was assessed using the same assay protocol as described in above. Data are shown in FIG. 6.

5′ GE and 3′ GP were observed to have similar activity by free uptake when conjugated to a 42mer, and 5′ GE was observed to have slightly higher free uptake than 3′ GP at higher doses when conjugated to a 30mer. The presence of a GalNAc linker does not appear to affect transfection editing, and 42mers were observed to edit better than 30mers both by transfection and free uptake.

### Comparison of 5′ Extended (GE) and 3′ Prolinol (GP) on a 42Mer and 30Mer (In Vivo)

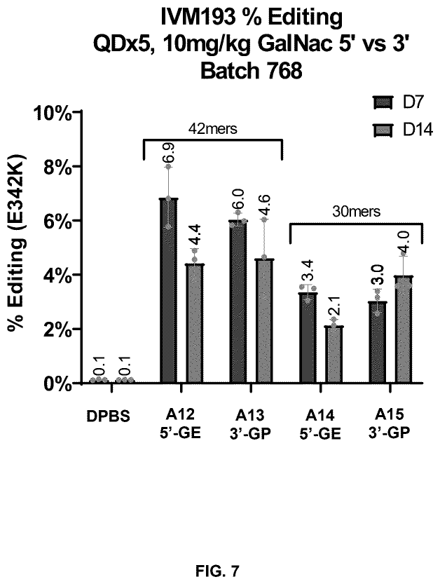

A study of in vivo editing of E342K by GalNAc oligonucleotides described herein in PiZ mice was undertaken. PiZ mice (n=3 per group) were treated with 42-mer or 30-mer oligonucleotides disclosed herein (5′ GE modified or 3′ GP modified) administered 5 QD×10 mg/kg (multiple doses), with takedowns at days 11 and 18. A summary of the study conditions is presented in Table 10, and data are shown in FIG. 7.

TABLE 10

| | | | Dosing | Dose | Dosing | Collection |
|---|---|---|---|---|---|---|
| Group | Animals | Treatment | Route | (mg/kg) | Regime | Day |
| 1 | 1-3 | DBPS | SC | n/a | QDx5 | 11 |
| | | (Untreated) | | | | |
| 2 | 4-6 | A12 5′-GE | SC | 10 | QDx5 | 11 |
| 3 | 7-9 | A13 3′-GP | SC | 10 | QDx5 | 11 |
| 4 | 10-12 | A14 5′ -GE | SC | 10 | QDx5 | 11 |
| 5 | 13-15 | A15 3′- GP | SC | 10 | QDx5 | 11 |
| 6 | 16-18 | DBPS | SC | n/a | QDx5 | 18 |
| | | (Untreated) | | | | |
| 7 | 19-21 | A12 5′-GE | SC | 10 | QDx5 | 18 |
| 8 | 22-24 | A13 3′-GP | SC | 10 | QDx5 | 18 |
| 9 | 25-27 | A14 5′ -GE | SC | 10 | QDx5 | 18 |
| 10 | 28-30 | A15 3′- GP | SC | 10 | QDx5 | 18 |

5′ GE vs 3′ GP did not appear to impact activity of the 42mer, and the 3′ GP on 30mer produced a slight increase in activity on day 14.

### Comparison of 3′ and 5′ Prolinol (GP), 5′ Extended (GE), and 5′ Decanol (GD) on a 30Mer (In Vitro)

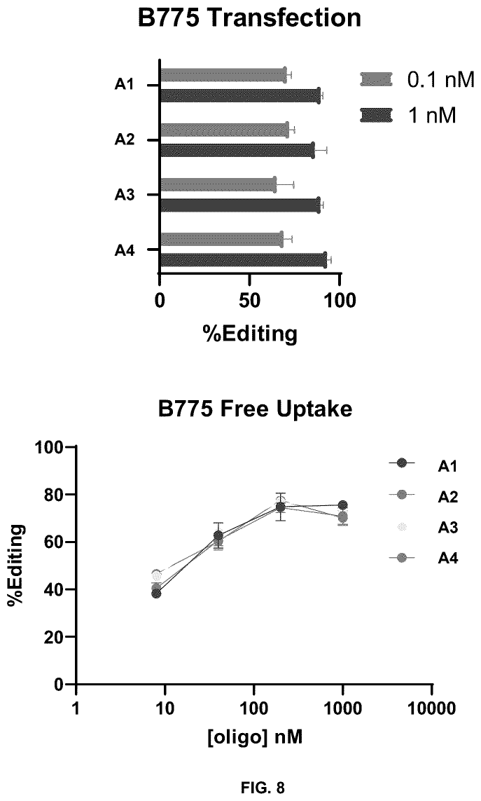

A study of in vitro and in vivo editing of E342K by GalNAc oligonucleotides described herein through transfection or free uptake was undertaken. Editing was assessed using the same assay protocol as described above. Data are shown in FIG. 8.

Little difference between GalNAc linkers as measured by overall editing by transfection and free uptake.

### Comparison of 3′ and 5′ Prolinol (GP), 5′ Extended (GE), and 5′ Decanol (GD) on a 30Mer (In Vivo)

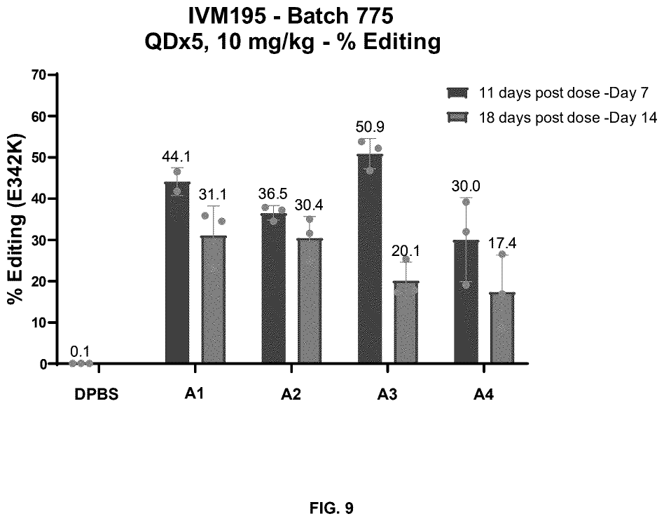

A study of in vivo editing of E342K by GalNAc oligonucleotides described herein in PiZ mice was undertaken. PiZ mice (n=27) were treated with 30-mer oligonucleotides disclosed herein (5′ GE modified, 3′ or 5′ GP modified, or 5′ GD modified) administered 5 QD×10 mg/kg (multiple doses), with takedowns at days 11 and 18. A summary of the study conditions is presented in Table 11, and data are shown in FIG. 9.

TABLE 11

| | | | Dosing | Dose | Collection |
|---|---|---|---|---|---|
| Group | Animals | Treatment | Route | (mg/kg) | Day |
| 1 | 1-3 | DPBS (Control) | SC | n/a | 11 |
| 2 | 4-6 | A1 | SC | 10 | 11 |
| 3 | 7-9 | A2 | SC | 10 | 11 |
| 4 | 10-12 | A3 | SC | 10 | 11 |
| 5 | 13-15 | A4 | SC | 10 | 11 |
| 6 | 16-18 | A1 | SC | 10 | 18 |
| 7 | 19-21 | A2 | SC | 10 | 18 |
| 8 | 22-24 | A3 | SC | 10 | 18 |
| 9 | 25-27 | A4 | SC | 10 | 18 |

Varying the Ga c linker an site of conjugation resulted in slightly different levels of editing. The extended linker (GE) appeared to result in more variable data at both 11 and 18 days post-last dose. Variability with the 5′ GE linker was also seen in vivo with an ACTB oligo (IVM184) as described in Example 2 herein.

### Example 4—Editing Efficiency of GalNAc Oligonucleotides on RAB7A

The uptake and editing efficiency of GalNAc oligonucleotides disclosed herein on the Rab7a gene in WT mice were studied in vivo. The RAB7A gene in humans encodes Ras-related protein Rab-7a, which is involved in endocytosis. Mutations of RAB7A are implicated in a variety of diseases and disorders, including melanomas, Charcot-Marie-Tooth neuropathy, hereditary motor and sensory neuropathy (HMSN), and peroneal muscular atrophy (PMA).

### Comparison of 3′ GP and 5′ GD on Rab7a Oligonucleotide Designs in WT and PiZ Mice

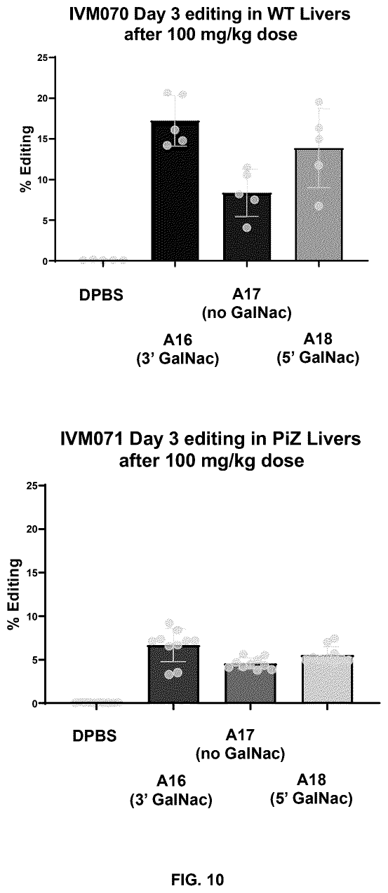

A study of in vivo editing of Rab7a by GalNAc oligonucleotides described herein in the liver tissue of WT and PiZ mice was undertaken. Mice were injected subcutaneously with oligonucleotides once at a dose of 100 mg/kg. Livers were isolated from mice three days after injection, and total RNA was isolated. The isolated RNA was used for cDNA synthesis using SuperScript IV VILO™ according to the manufacturer's instructions (Life Technologies). Ten μl of the cDNA was used for Next Generation Sequencing (NGS), Amplicon Sequencing by Quintara Biosciences. Percent editing of the site of interest was quantified as a percentage of the number of edited nucleotides based on NGS counts. Each oligonucleotide was assayed in at least three replicates. Primer sequences used for NGS are shown below in Table 12. For these oligonucleotides, 3′ and 5′ GalNAc conjugations showed comparable activity, which were both higher than the unconjugated control oligonucleotide. Data are shown in FIG. 10.

TABLE 12
PCR and sequencing primers

| | |
|---|---|
| Forward primer | gaattcctggattgtgtgtttaagt |
| Mouse RAB7A Site 1 | (SEQ ID NO: 75) |
| Reverse primer | cagaagggaactgtctagttcagt |
| Mouse RAB7A Site 1 | (SEQ ID NO: 76) |

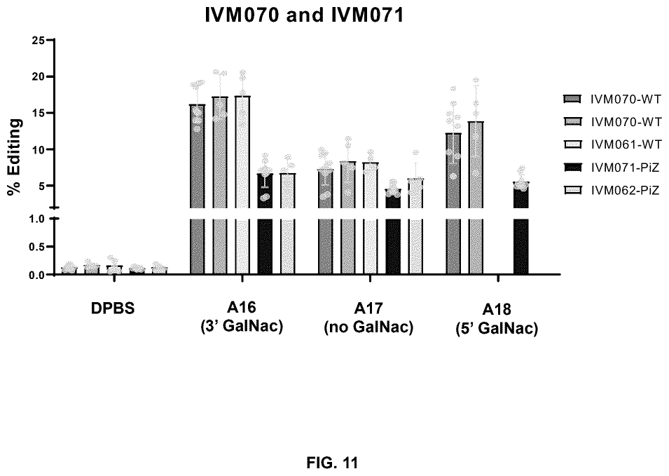

For the asymmetrical antisense oligonucleotide (ASO), 3′ vs 5′ GalNAc conjugation was observed to have similar activity. Data for 3′ and non GalNAc formulations are similar to IVM060 and IVM061, indicating reproducibility across studies (data shown in FIG. 11).

### In Vitro Evaluation of 5′ Decanol (GD) and 5′ and 3′ Prolinol (GP) in Primary Mouse Hepatocytes (PMH)

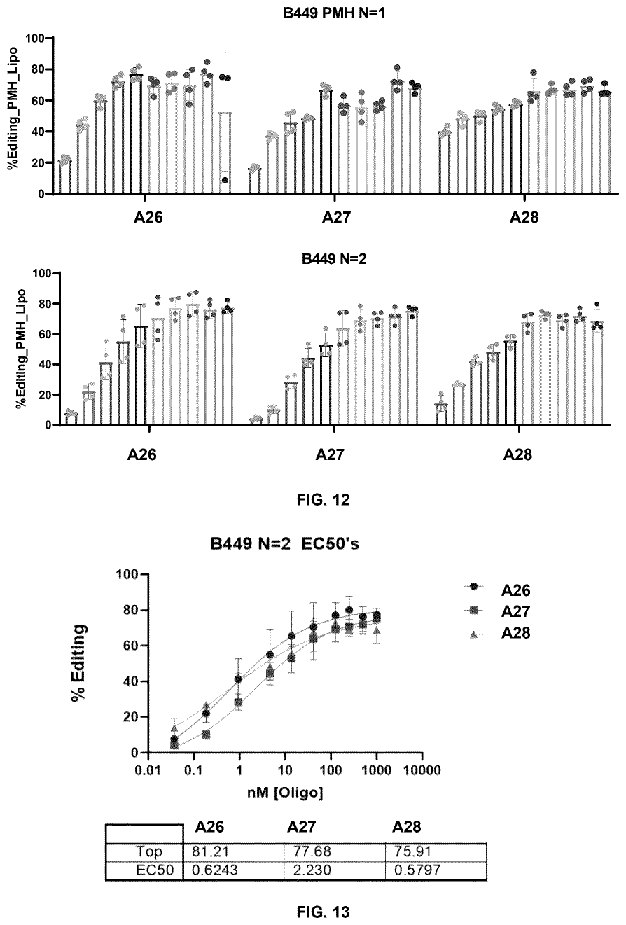

A study of in vitro editing of Rab7a by varying concentrations of GalNAc oligonucleotides described herein as measured in primary mouse hepatocytes (PMHs) was undertaken. Mouse hepatocytes were thawed in a 37° C. water bath in a 50 ml tube of Lonza rodent cryopreserved hepatocyte thawing media (MCRT50-Lonza). After centrifugation at 100×g for 5 minutes, supernatant was aspirated and the cell pellet is resuspended in Lonza plating media (Lonza). Cells were plated on 96-well collagen-coated tissue culture plates at 20000 cells/well. Cells were transferred to incubator (37° C.), 4 to 6 hours later media was changed to Lonza maintenance media (CC-3198, LonzA) and cells were transfected with ASOs at desired concentrations, with and without RNAiMax (Life Technologies, CA) according to manufacturer's protocol and transferred to a 37° C. incubator. Primers for NGS are shown above for Rab7a in Table 12. The study was run twice (N=1 and N=2), and data are shown in FIG. 12.

For the first study run (N=1), slightly less editing at select doses was observed between no GalNAc vs 3′ GalNAc oligos (7254-4 and 10300-2). 5′ GalNAc was observed to have higher editing at lowest doses compared to no GalNAc and 3′ GalNAc. For the second study run (N=2), very similar editing was observed overall across oligos with 3′ vs 5′ vs no GalNAc. Select doses were observed to show statistically different editing (e.g. 0.185 nM A28 vs. A30), but overall there were no large differences in editing based on presence or position of GalNAc.

EC50 were generated based on the above concentration data. Curves are shown in FIG. 13.

EC50s were found to be somewhat better for GalNAc vs no modification.

In vivo evaluation of 5′ Decanol (GD) and 5′ and 3′ Prolinol (GP) Oligonucleotide Rab7a Editing in Mice

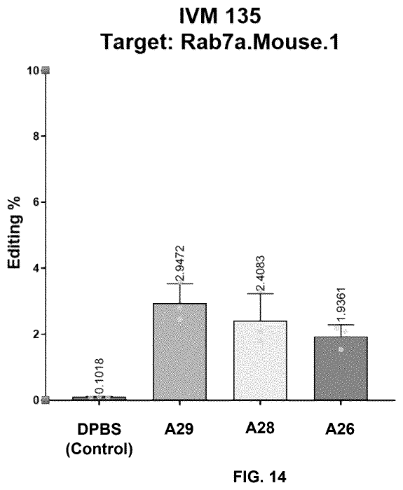

A study of in vivo editing of Rab7a by GalNAc oligonucleotides described herein in mice was undertaken. Mice (n=3 per group) were treated with 54-mer oligonucleotides disclosed herein (5′ GD modified or 3′ GP modified) administered at 50 mg/kg (single dose daily), with collection at day 3. A summary of the study conditions is presented in Table 13, and data are shown in FIG. 14.

TABLE 13

| | | | Dose | Collection |
|---|---|---|---|---|
| Group | Animals | Treatment | (mg/kg) | Day |
| 1 | 1-3 | N/A | 0 | QDx1 |
| 2 | 4-6 | A29 | 50 | QDx1 |
| 3 | 7-9 | A30 | 50 | QDx1 |
| 4 | 10-12 | A31 | 50 | QDx1 |

Editing was comparable for the two 5′ and one 3′ GalNAc-conjugated oligonucleotides. While editing was low (less than 5%) for all oligonucleotides, the oligonucleotide with a 5′ GD showed the highest editing at 2.9%, compared to 1.9% for the 3′ GP and 2.4% for the 5′ GP.

### Example 5—Detection of GalNAc Oligonucleotide Metabolites in Liver

Liver tissue from the in vivo evaluation discussed in Example 4 herein were analyzed for the presence of GalNAc-containing metabolites. Day 3 liver samples were processed via liquid-liquid extraction (LLE) and eluent was analyzed by liquid chromatography high resolution mass spectrometry (LC-HRMS). In short, 6 μL of stop buffer containing 1 mg/mL of proteinase K was added per 1 mg of liver tissue prior to homogenization at 2,100 rpm for 30 seconds at room temperature. Individual animal samples were pooled, at equivalent volumes, by group, internal standard (IS) was added and samples were incubated at 60° C. for 60 min on a plate shaker set to 300 rpm followed by LLE. After LLE, samples were dried to ˜200 μL and analyzed using liquid chromatography (Thermo Vanquish UHPLC) combined with mass spectrometry detection on a Thermo QExactive+ by electrospray ionization (ESI). Samples were injected (5 or 10 μL) and separated using an Acquity UPLC BEH C18 Column, 1.7 μm, 2.1×100 mm (Waters, Cat. 186002352) maintained at 80° C. Mobile phase A was 10 mM TEA and 100 mM hexafluoroisopropanol in water and mobile phase B was methanol. A gradient of 5-30% mobile phase B over 10.5. minutes was employed at 0.25 mL/min. The ESI source was operated in negative ion mode, with full scan, using spray voltage=3100 V, sheath gas flow=30 units, auxiliary gas flow=25 units, sweep gas flow=0 units, and capillary temperature=320° C. Thermo BioPharma Finder software was used to deconvolute the signal, assignment of structural modifications was made based on deconvoluted exact mass, retention time and operator interpretation.

No GalNAc-containing metabolites were observed in liver tissue from mice administered 5′ GD or 5′ GP oligonucleotides. Livers from mice administered 3′ GP oligonucleotides exhibited a normal distribution of 3′ GalNAc metabolites as chemical moieties are cleaved from 3′ GalNAc over time.

The metabolite identification findings for 5′ and 3′ GP highlight fundamental differences in GalNAc stability based on conjugation site.

What is claimed is:

1. A modified oligonucleotide having a structure of Formula (I): 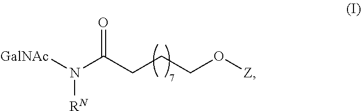
wherein
GalNAc has a structure of 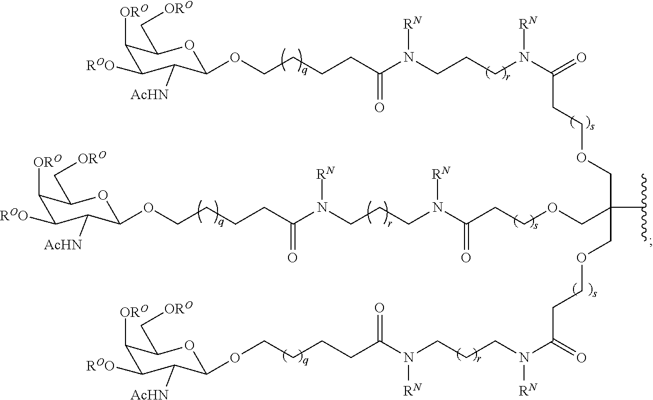
each R^{o} is independently H or C₁₋₃alkyl;
each R^{N} is independently H or C₁₋₃alkyl;
each q, r, and s is independently 0, 1, or 2;
t is an integer from 1 to 20; and
Z is an oligonucleotide moiety.

2. The modified oligonucleotide of claim 1, wherein each R^{o} is H.

3. The modified oligonucleotide of claim 1, wherein each R^{N} is H.

4. The modified oligonucleotide of claim 1, wherein each q is 1.

5. The modified oligonucleotide of claim 1, wherein each r is 1.

6. The modified oligonucleotide of claim 1, wherein each s is 1.

7. The modified oligonucleotide of claim 1, wherein GalNAc has a structure of

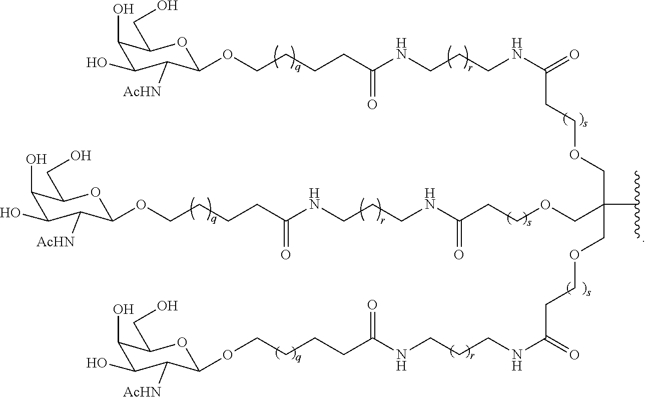

8. The modified oligonucleotide of claim 1, having a structure of Formula (IA):

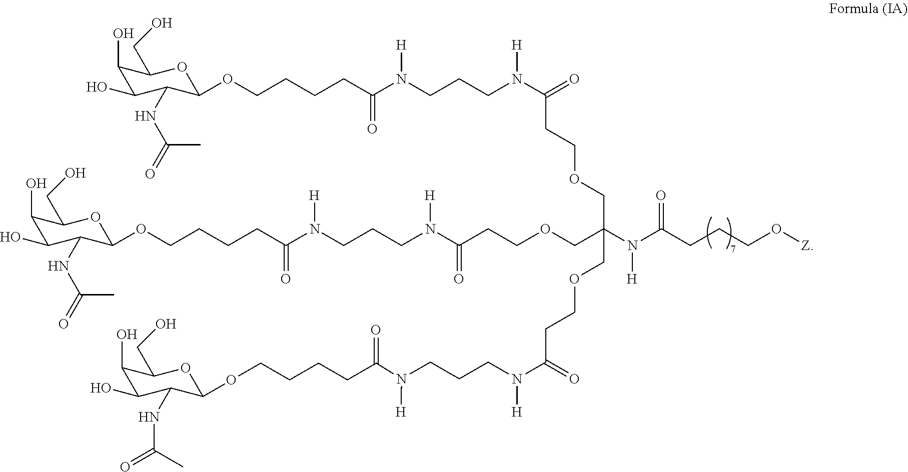

9. The modified oligonucleotide of claim 1, wherein Z is modified at the 5′ end.

10. The modified oligonucleotide of claim 1, wherein Z is modified at the 3′ end.

11. The modified oligonucleotide of claim 1, wherein Z comprises 10 to 300 nucleotides, 25-100 nucleotides, or 30-50 nucleotides, each nucleotide comprising a sugar moiety, a nucleobase, and an internucleotide linkage.

12. The modified oligonucleotide of claim 11, wherein at least one sugar moiety is β-D-2′,3′-dideoxyglucopyranose (β-homo-RNA), 2′-methoxyribose, 2′-methoxyethylribose (2′-MOE-ribose), 2′-fluororibose, 5′-methyl-2′deoxyribose, 2′-deoxy-2′-fluororibose, 2′-fluoroarabinose, 2-methoxy-arabinose, 2′deoxyribose, a locked nucleic acid (LNA), constrained ethyl (cEt), bridged nucleic acid (BNA), or deoxyhexose.

13. The modified oligonucleotide of claim 12, wherein at least 10% of the sugar moieties are β-D-2′,3′-dideoxyglucopyranose (β-homo-RNA), 2′-methoxyribose, 2′-methoxyethylribose (2′-MOE-ribose), 2′-fluororibose, 5′-methyl-2′deoxyribose, 2′-deoxy-2′-fluororibose, 2′-fluoroarabinose, 2-methoxy-arabinose, 2′deoxyribose, a locked nucleic acid (LNA), constrained ethyl (cEt), bridged nucleic acid (BNA), or deoxyhexose.

14. The modified oligonucleotide of claim 11, wherein at least one internucleotide linkage comprises a phosphorothioate or phosphoramidate linkage.

15. The modified oligonucleotide of claim 14, wherein at least 10% of the internucleotide linkages of the passenger oligonucleotide comprise phosphorothioate or phosphoramidate linkages.

16. The modified oligonucleotide of claim 14, wherein the phosphoroamidate is a mesyl phosphoramidate with mesyl azide (PA-1), an isopropylsulfonylphosporamidate, or a cyclopropylsulfonylphosphoramidate.

17. The modified oligonucleotide of claim 11, wherein at least one nucleobase is a hypoxanthine or a 5′-methylcytosine.

18. The modified oligonucleotide of claim 1, having a sequence that is sufficiently complementary to a target RNA.

19. The modified oligonucleotide of claim 18, capable of binding and recruiting an ADAR enzyme to perform editing on a target adenosine of the target RNA.

20. A formulation comprising the modified oligonucleotide of claim 1 and a pharmaceutically acceptable excipient.
